SEQUENCE LISTING (22 sequences)

SEQ ID NO: 1 - DNA, 2264 bp, Human
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa

SEQ ID NO: 2 - DNA, 2032 bp, artificial sequence
  Synthesized
tccatattgtgcttccaccactgccaataacaaaataactagcaaccatgaagtgggtggaatcaatttttttaattttcctactaaattttactgaatccagaacactgcatagaaatgaatatggaatagcttccatattggattcttaccaatgtactgcagagataagtttagctgacctggctaccatattttttgcccagtttgttcaagaagccacttacaaggaagtaagcaaaatggtgaaagatgcattgactgcaattgagaaacccactggagatgaacagtcttcagggtgtttagaaaaccagctacctgcctttctggaagaactttgccatgagaaagaaattttggagaagtacggacattcagactgctgcagccaaagtgaagagggaagacataactgttttcttgcacacaaaaagcccactccagcatcgatcccacttttccaagttccagaacctgtcacaagctgtgaagcatatgaagaagacagggagacattcatgaacaaattcatttatgagatagcaagaaggcatcccttcctgtatgcacctacaattcttctttgggctgctcgctatgacaaaataattccatcttgctgcaaagctgaaaatgcagttgaatgcttccaaacaaaggcagcaacagttacaaaagaattaagagaaagcagcttgttaaatcaacatgcatgtgcagtaatgaaaaattttgggacccgaactttccaagccataactgttactaaactgagtcagaagtttaccaaagttaattttactgaaatccagaaactagtcctggatgtggcccatgtacatgagcactgttgcagaggagatgtgctggattgtctgcaggatggggaaaaaatcatgtcctacatatgttctcaacaagacactctgtcaaacaaaataacagaatgctgcaaactgaccacgctggaacgtggtcaatgtataattcatgcagaaaatgatgaaaaacctgaaggtctatctccaaatctaaacaggtttttaggagatagagattttaaccaattttcttcaggggaaaaaaatatcttcttggcaagttttgttcatgaatattcaagaagacatcctcagcttgctgtctcagtaattctaagagttgctaaaggataccaggagttattggagaagtgtttccagactgaaaaccctcttgaatgccaagataaaggagaagaagaattacagaaatacatccaggagagccaagcattggcaaagcgaagctgcggcctcttccagaaactaggagaatattacttacaaaatgcgtttctcgttgcttacacaaagaaagccccccagctgacctcgtcggagctgatggccatcaccagaaaaatggcagccacagcagccacttgttgccaactcagtgaggacaaactattggcctgtggcgagggagcggctgacattattatcggacacttatgtatcagacatgaaatgactccagtaaaccctggtgttggccagtgctgcacttcttcatatgccaacaggaggccatgcttcagcagcttggtggtggatgaaacatatgtccctcctgcattctctgatgacaagttcattttccataaggatctgtgccaagctcagggtgtagcgctgcaaacgatgaagcaagagtttctcattaaccttgtgaagcaaaagccacaaataacagaggaacaacttgaggctgtcattgcagatttctcaggcctgttggagaaatgctgccaaggccaggaacaggaagtctgctttgctgaagagggacaaaaactgatttcaaaaactcgtgctgctttgggagtttaaattacttcaggggaagagaagacaaaacgagtctttcattcggtgtgaacttttctctttaattttaactgatttaacactttttgtgaattaatgaaatgataaagacttttatgtgagatttccttatcacagaaataaaatatctccaaatg

SEQ ID NO: 3 - DNA, 2024 bp, artificial sequence
  Synthesized
atattaagtaaactttagtgaggaacagcagtggaaaataatctatataccttggctcttttgcagtttgacaaagttaatgattaaaatctcctagattttccactacagtatccccagggtgtctatttaccttgattgatattattttatctcttttgggccaaagataacagccccttgcttctgtgtttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaaaaaaaaaaaaaaaaaaaaaaaaaaaaaaaaaa

SEQ ID NO: 4 - DNA, 1997 bp, artificial sequence
  Synthesized
actttcttttgtaaatgtggtttctacaaagatgaaactactaaaacttacaggttttatttttttcttgttttttttgactgaatccctaaccctgcccacacaacctcgggatatagagaacttcaatagtactcaaaaatttatagaagataatattgaatacatcaccatcattgcatttgctcagtatgttcaggaagcaacctttgaagaaatggaaaagctggtgaaagacatggtagaatacaaagacagatgtatggctgacaagacgctcccagagtgttcaaaattacctaataatgttttacaggaaaaaatatgtgctatggaggggctgccacaaaagcataatttctcacactgctgcagtaaggttgatgctcaaagaagactctgtttcttctataacaagaaatctgatgtgggatttctgcctcctttccctaccctggatcccgaagagaaatgccaggcttatgaaagtaacagagaatcccttttaaatcactttttatatgaagttgccagaaggaacccatttgtcttcgcccctacacttctaactgttgctgttcattttgaggaggtggccaaatcatgttgtgaagaacaaaacaaagtcaactgccttcaaacaagggcaatacctgtcacacaatatttaaaagcattttcttcttatcaaaaacatgtctgtggggcacttttgaaatttggaaccaaagttgtacactttatatatattgcgatactcagtcaaaaattccccaagattgaatttaaggagcttatttctcttgtagaagatgtttcttccaactatgatggatgctgtgaaggggatgttgtgcagtgcatccgtgacacgagcaaggttatgaaccatatttgttcaaaacaagattctatctccagcaaaatcaaagagtgctgtgaaaagaaaataccagagcgcggccagtgcataattaactcaaacaaagatgatagaccaaaggatttatctctaagagaaggaaaatttactgacagtgaaaatgtgtgtcaagaacgagatgctgacccagacaccttctttgcgaagtttacttttgaatactcaaggagacatccagacctgtctataccagagcttttaagaattgttcaaatatacaaagatctcctgagaaattgctgcaacacagaaaaccctccaggttgttaccgttacgcggaagacaaattcaatgagacaactgagaaaagcctcaagatggtacaacaagaatgtaaacatttccagaatttggggaaggatggtttgaaataccattacctcatcaggctcacgaagatagctccccaactctccactgaagaactggtgtctcttggcgagaaaatggtgacagctttcactacttgctgtacgctaagtgaagagtttgcctgtgttgataatttggcagatttagtttttggagagttatgtggagtaaatgaaaatcgaactatcaaccctgctgtggaccactgctgtaaaacaaactttgccttcagaaggccctgctttgagagtttgaaagctgataaaacatatgtgcctccacctttctctcaagatttatttacctttcacgcagacatgtgtcaatctcagaatgaggagcttcagaggaagacagacaggtttcttgtcaacttagtgaagctgaagcatgaactcacagatgaagagctgcagtctttgtttacaaatttcgcaaatgtagtggataagtgctgcaaagcagagagtcctgaagtctgctttaatgaagagagtccaaaaattggcaactgaagccagctgctggagatatgtaaagaaaaaagcaccaaagggaaggcttcctatctgtgtggtgatgaatcgcatttcctgagaacaaaataaaaggatttttctgtaactgtcacctgaaataatacattgcagcaagcaataaacacaacattttgtaaagtta

SEQ ID NO: 5 - protein, 1652 aa, Artificial Sequence
  Synthesized
METLYSARGVALLEVALLELELEALAVALALAPHEGLYHISALALEGLARGGLYARGASPTYRGLLYSASNLYSVALCYSLYSGLPHESERHISLEGLYLYSGLASPPHETHRSERLESERLEVALLETYRSERARGLYSPHEPRSERGLYTHRPHEGLGLNVALSERGLNLEVALLYSGLVALVALSERLETHRGLALACYSVALALAGLGLYALAASPPRASPCYSTYRASPTHRARGTHRSERALALESERALALYSSERCYSGLSERASNSERPRPHEPRVALHISPRGLYTHRALAGLCYSCYSTHRLYSGLGLYLEGLARGLYSLECYSMETALAALALELYSHISGLNPRGLNGLPHEPRTHRTYRVALGLPRTHRASNASPGLILECYSGLALAPHEARGLYSASPPRLYSGLTYRALAASNGLNPHEMETTRPGLTYRSERTHRASNTYRGLYGLNALAPRLESERLELEVALSERTYRTHRLYSSERTYRLESERMETVALGLYSERCYSCYSTHRSERALASERPRTHRVALCYSPHELELYSGLARGLEGLNLELYSHISLESERLELETHRTHRLESERASNARGVALCYSSERGLNTYRALAALATYRGLYGLLYSLYSSERARGLESERASNLEILELYSLEALAGLNLYSVALPRTHRALAASPLEGLASPVALLEPRLEALAGLASPILETHRASNILELESERLYSCYSCYSGLSERALASERGLASPCYSMETALALYSGLLEPRGLHISTHRVALLYSLECYSASPASNGLNASPTHRLYSASNSERLYSPHEGLASPCYSCYSGLNGLLYSTHRALAMETASPVALPHEVALCYSTHRTYRPHEMETPRALAALAGLNLEPRGLLEPRASPVALGLLEPRTHRASNLYSASPVALCYSASPPRGLYASNTHRLYSVALMETASPLYSTYRTHRPHEGLLESERARGARGTHRHISLEPRGLVALPHELESERLYSVALLEGLPRTHRLELYSSERLEGLYGLCYSCYSASPVALGLASPSERTHRTHRCYSPHEASNALALYSGLYPRLELELYSLYSGLLESERSERPHEILEASPLYSGLYGLNGLLECYSALAASPTYRSERGLASNTHRPHETYRTYRLEGLNASNALAPHELEVALALATYRTHRLYSLYSALAPRGLNLETHRSERSERGLLEMETALAILETHRARGLYSMETALAALATHRALAALATHRCYSCYSGLNLESERGLASPLYSLELEALACYSGLYGLGLYALAALAASPILEILEILEGLYHISLECYSILELEHISGLMETTHRPRVALSERASPARGVALTHRGLNCYSCYSTHRSERSERTYRALAASNARGARGPRCYSPHESERSERLEGLVALASPGLTHRTYRVALPRLYSGLPHESERASPASPLYSPHETHRPHEHISSERASPLECYSGLNALAGLNGLYVALALALEGLNTHRMETLYSGLNGLPHELEILEASNLEVALLYSHISLYSPRLYSILETHRGLGLGLNLEGLALAVALILEALAASPPHESERGLYLELEGLLYSCYSCYSGLNGLYGLNGLGLNGLVALCYSPHEALAGLGLGLYTRPLYSLEILESERLYSTHRARGALAALALEGLYVAL

SEQ ID NO: 6 - protein, 1687 aa, artificial sequence
  Synthesized
METLYSTRPVALGLSERILEPHELEILEPHELELEASNPHETHRGLSERARGTHRLEHISARGASNGLTYRGLYILEALASERILELEASPSERTYRGLNCYSTHRALAGLILESERLEALAASPLEALATHRILEPHEPHEALAGLNPHEVALGLNGLALATHRTYRLYSGLVALSERLYSMETVALLYSASPALALETHRALAILEGLLYSPRTHRGLYASPGLGLNSERSERGLYCYSLEGLASNGLNLEPRALAPHELEGLGLLECYSHISGLLYSGLILELEGLLYSTYRGLYHISSERASPCYSCYSSERGLNSERGLGLGLYARGHISASNCYSPHELEALAHISLYSLYSPRTHRPRALASERILEPRLEPHEGLNVALPRGLPRVALTHRSERCYSGLALATYRGLGLASPARGGLTHRPHEMETASNLYSPHEILETYRGLILEALAARGARGHISPRPHELETYRALAPRTHRILELELETRPALAALAARGTYRASPLYSILEILEPRSERCYSCYSLYSALAGLASNALAVALGLCYSPHEGLNTHRLYSALAALATHRVALTHRLYSGLLEARGGLSERSERLELEASNGLNHISALACYSALAVALMETLYSASNPHEGLYTHRARGTHRPHEGLNALAILETHRVALTHRLYSLESERGLNLYSPHETHRLYSVALASNPHETHRGLILEGLNLYSLEVALLEASPVALALAHISVALHISGLHISCYSCYSARGGLYASPVALLEASPCYSLEGLNASPGLYGLLYSILEMETSERTYRILECYSSERGLNGLNASPTHRLESERASNLYSILETHRGLCYSCYSLYSLETHRTHRLEGLARGGLYGLNCYSILEILEHISALAGLASNASPGLLYSPRGLGLYLESERPRASNLEASNARGPHELEGLYASPARGASPPHEASNGLNPHESERSERGLYGLLYSASNILEPHELEALASERPHEVALHISGLTYRSERARGARGHISPRGLNLEALAVALSERVALILELEARGVALALALYSGLYTYRGLNGLLELEGLLYSCYSPHEGLNTHRGLASNPRLEGLCYSGLNASPLYSGLYGLGLGLLEGLNLYSTYRILEGLNGLSERGLNALALEALALYSARGSERCYSGLYLEPHEGLNLYSLEGLYGLTYRTYRLEGLNASNALAPHELEVALALATYRTHRLYSLYSALAPRGLNLETHRSERSERGLLEMETALAILETHRARGLYSMETALAALATHRALAALATHRCYSCYSGLNLESERGLASPLYSLELEALACYSGLYGLGLYALAALAASPILEILEILEGLYHISLECYSILEARGHISGLMETTHRPRVALASNPRGLYVALGLYGLNCYSCYSTHRSERSERTYRALAASNARGARGPRCYSPHESERSERLEVALVALASPGLTHRTYRVALPRPRALAPHESERASPASPLYSPHEILEPHEHISLYSASPLECYSGLNALAGLNGLYVALALALEGLNTHRMETLYSGLNGLPHELEILEASNLEVALLYSGLNLYSPRGLNILETHRGLGLGLNLEGLALAVALILEALAASPPHESERGLYLELEGLLYSCYSCYSGLNGLYGLNGLGLNGLVALCYSPHEALAGLGLGLYGLNLYSLEILESERLYSTHRARGALAALALEGLYVAL

SEQ ID NO: 7 - protein, 1296 aa, artificial sequence
  Synthesized
METLYSARGVALLEVALLELELEALAVALALAPHEGLYHISALALEGLARGGLYARGASPTYRGLLYSASNLYSVALCYSLYSGLPHESERHISLEGLYLYSGLASPPHETHRSERLESERLEVALLETYRSERARGLYSPHEPRSERGLYTHRPHEGLGLNVALSERGLNLEVALLYSGLVALVALSERLETHRGLALACYSCYSALAGLGLYALAASPPRASPCYSTYRASPTHRARGTHRSERALALESERALALYSSERCYSGLSERASNSERPRPHEPRVALHISPRGLYTHRALAGLCYSCYSTHRLYSGLGLYLEGLARGLYSLECYSMETALAALALELYSHISGLNPRGLNGLPHEPRTHRTYRVALGLPRTHRASNASPGLILECYSGLALAPHEARGLYSASPPRLYSGLTYRALAASNGLNPHEMETTRPGLTYRSERTHRASNTYRGLYGLNALAPRLESERLELEVALSERTYRTHRLYSSERTYRLESERMETVALGLYSERCYSCYSTHRSERALASERPRTHRVALCYSPHELELYSGLARGLEGLNLELYSHISLESERLELETHRTHRLESERASNARGVALCYSSERGLNTYRALAALATYRGLYGLLYSLYSSERARGLESERASNLEILELYSLEALAGLNLYSVALPRTHRALAASPLEGLASPVALLEPRLEALAGLASPILETHRASNILELESERLYSCYSCYSGLSERALASERGLASPCYSMETALALYSGLLEPRGLHISTHRVALLYSLECYSASPASNLESERTHRLYSASNSERLYSPHEGLASPCYSCYSGLNGLLYSTHRALAMETASPVALPHEVALCYSTHRTYRPHEMETPRALAALAGLNLEPRGLLEPRASPVALGLLEPRTHRASNLYSASPVALCYSASPPRGLYASNTHRLYSVALMETASPLYSTYRTHRPHEGLLESERARGARGTHRHISLEPRGLVALPHELESERLYSVALLEGLPRTHRLELYSSERLEGLYGLCYSCYSASPVALGLASPSERTHRTHRCYSPHEASNALALYSGLYPRLELELYSLYSGLLESERSERPHEILEASPLYSGLYGLNGLLECYSALAASPTYRSERGLASNTHRPHETHRGLTYRLYSLYSLYSLEALAGLARGLELYSALALYSLEPRASPALATHRPRLYSGLLEALALYSLEVALASNLYSARGSERASPPHEALASERASNCYSCYSSERILEASNSERPRPRLETYRCYSASPSERGLILEASPALAGLLELYSASNILELE

SEQ ID NO: 8 - DNA, 19 bp, artificial sequence
  Synthesized
tctttttctctttagctcg

SEQ ID NO: 9 - DNA, 19 bp, artificial sequence
  Synthesized
tacgtgcatctcgacgaaa

SEQ ID NO: 10 - protein, 1693 aa, artificial sequence
  Synthesized
METLYSLELELYSLETHRGLYPHEILEPHEPHELEPHEPHELETHRGLSERLETHRLEPRTHRGLNPRARGASPILEGLASNPHEASNSERTHRGLNLYSPHEILEGLASPASNILEGLTYRILETHRILEILEALAPHEALAGLNTYRVALGLNGLALATHRPHEGLGLMETGLLYSLEVALLYSASPMETVALGLTYRLYSASPARGCYSMETALAASPLYSTHRLEPRGLCYSSERLYSLEPRASNASNVALLEGLNGLLYSILECYSALAMETGLGLYLEPRGLNLYSHISASNPHESERHISCYSCYSSERLYSVALASPALAGLNARGARGLECYSPHEPHETYRASNLYSLYSSERASPVALGLYPHELEPRPRPHEPRTHRLEASPPRGLGLLYSCYSGLNALATYRGLSERASNARGGLSERLELEASNHISPHELETYRGLVALALAARGARGASNPRPHEVALPHEALAPRTHRLELETHRVALALAVALHISPHEGLGLVALALALYSSERCYSCYSGLGLGLNASNLYSVALASNCYSLEGLNTHRARGALAILEPRVALTHRGLNTYRLELYSALAPHESERSERTYRGLNLYSHISVALCYSGLYALALELELYSPHEGLYTHRLYSVALVALHISPHEILETYRILEALAILELESERGLNLYSPHEPRLYSILEGLPHELYSGLLEILESERLEVALGLASPVALSERSERASNTYRASPGLYCYSCYSGLGLYASPVALVALGLNCYSILEARGASPTHRSERLYSVALMETASNHISILECYSSERLYSGLNASPSERILESERSERLYSILELYSGLCYSCYSGLLYSLYSILEPRGLARGGLYGLNCYSILEILEASNSERASNLYSASPASPARGPRLYSASPLESERLEARGGLGLYLYSPHETHRASPSERGLASNVALCYSGLNGLARGASPALAASPPRASPTHRPHEPHEALALYSPHETHRPHEGLTYRSERARGARGHISPRASPLESERILEPRGLLELEARGILEVALGLNILETYRLYSASPLELEARGASNCYSCYSASNTHRGLASNPRPRGLYCYSTYRARGTYRALAGLASPLYSPHEASNGLTHRTHRGLLYSSERLELYSMETVALGLNGLNGLCYSLYSHISPHEGLNASNLEGLYLYSTYRTYRLEGLNASNALAPHELEVALALATYRTHRLYSLYSALAPRGLNLETHRSERSERGLLEMETALAILETHRARGLYSMETALAALATHRALAALATHRCYSCYSGLNLESERGLASPLYSLELEALACYSGLYGLGLYALAALAASPILEILEILEGLYHISLECYSILELEHISGLMETTHRPRVALSERASPARGVALTHRGLNCYSCYSTHRSERSERTYRALAASNARGARGPRCYSPHESERSERLEGLVALASPGLTHRTYRVALPRLYSGLPHESERASPASPLYSPHETHRPHEHISSERASPLECYSGLNALAGLNGLYVALALALEGLNTHRMETLYSGLNGLPHELEILEASNLEVALLYSHISLYSPRLYSILETHRGLGLGLNLEGLALAVALILEALAASPPHESERGLYLELEGLLYSCYSCYSGLNGLYGLNGLGLNGLVALCYSPHEALAGLGLGLYTRPLYSLEILESERLYSTHRARGALAALALEGLYVAL

SEQ ID NO: 11 - protein, 2928 aa, artificial sequence
  Synthesized
METLYSTRPVALTHRPHEILESERLELEPHELEPHESERSERALATYRSERVALPHELEASPHISGLASNALAASNLYSILELEASNARGPRLYSARGTYRASNSERGLYLYSLEGLGLPHEVALGLNGLYASNLEGLARGGLCYSMETGLGLLYSCYSSERPHEGLGLALAARGGLVALPHEGLASNTHRGLARGTHRTHRGLPHETRPLYSGLNTYRVALASPGLYASPGLNCYSGLSERASNPRCYSLEASNGLYGLYSERCYSLYSASPASPILEASNSERTYRGLCYSTRPCYSPRPHEGLYPHEGLGLYLYSASNCYSGLLEASPVALTHRCYSASNILELYSASNGLYARGCYSGLGLNPHECYSLYSASNSERALAASPASNLYSVALVALCYSSERCYSTHRGLGLYTYRARGLEALAGLASNGLNLYSSERCYSGLPRALAVALPRPHEPRCYSGLYARGVALSERVALSERGLNTHRSERLYSLETHRARGALAGLTHRVALPHEPRASPVALASPTYRVALASNSERTHRGLALAGLTHRILELEASPASNILETHRGLNSERTHRGLNSERPHEASNASPPHETHRARGVALVALGLYGLYGLASPALALYSPRGLYGLNPHEPRTRPGLNVALVALLEASNGLYLYSVALASPALAPHECYSGLYGLYSERILEVALASNGLLYSTRPILEVALTHRALAALAHISCYSVALGLTHRGLYVALLYSILETHRVALVALALAGLYGLHISASNILEGLGLTHRGLHISTHRGLGLNLYSARGASNVALILEARGILEILEPRHISHISASNTYRASNALAALAILEASNLYSTYRASNHISASPILEALALELEGLLEASPGLPRLEVALLEASNSERTYRVALTHRPRILECYSILEALAASPLYSGLTYRTHRASNILEPHELELYSPHEGLYSERGLYTYRVALSERGLYTRPGLYARGVALPHEHISLYSGLYARGSERALALEVALLEGLNTYRLEARGVALPRLEVALASPARGALATHRCYSLEARGSERTHRLYSPHETHRILETYRASNASNMETPHECYSALAGLYPHEHISGLGLYGLYARGASPSERCYSGLNGLYASPSERGLYGLYPRHISVALTHRGLVALGLGLYTHRSERPHELETHRGLYILEILESERTRPGLYGLGLCYSALAMETLYSGLYLYSTYRGLYILETYRTHRLYSVALSERARGTYRVALASNTRPILELYSGLLYSTHRLYSLETHRGLALAALAALALYSGLALAALAALALYSGLALAALAALALYSGLALAALAALALYSGLALAALAALALYSGLLYSASNLYSVALCYSLYSGLPHESERHISLEGLYLYSGLASPPHETHRSERLESERLEVALLETYRSERARGLYSPHEPRSERGLYTHRPHEGLGLNVALSERGLNLEVALLYSGLVALVALSERLETHRGLALACYSVALALAGLGLYALAASPPRASPCYSTYRASPTHRARGTHRSERALALESERALALYSSERCYSGLSERASNSERPRPHEPRVALHISPRGLYTHRALAGLCYSCYSTHRLYSGLGLYLEGLARGLYSLECYSMETALAALALELYSHISGLNPRGLNGLPHEPRTHRTYRVALGLPRTHRASNASPGLILECYSGLALAPHEARGLYSASPPRLYSGLTYRALAASNGLNPHEMETTRPGLTYRSERTHRASNTYRGLYGLNALAPRLESERLELEVALSERTYRTHRLYSSERTYRLESERMETVALGLYSERCYSCYSTHRSERALASERPRTHRVALCYSPHELELYSGLARGLEGLNLELYSHISLESERLELETHRTHRLESERASNARGVALCYSSERGLNTYRALAALATYRGLYGLLYSLYSSERARGLESERASNLEILELYSLEALAGLNLYSVALPRTHRALAASPLEGLASPVALLEPRLEALAGLASPILETHRASNILELESERLYSCYSCYSGLSERALASERGLASPCYSMETALALYSGLLEPRGLHISTHRVALLYSLECYSASPASNGLNASPTHRLYSASNSERLYSPHEGLASPCYSCYSGLNGLLYSTHRALAMETASPVALPHEVALCYSTHRTYRPHEMETPRALAALAGLNLEPRGLLEPRASPVALGLLEPRTHRASNLYSASPVALCYSASPPRGLYASNTHRLYSVALMETASPLYSTYRTHRPHEGLLESERARGARGTHRHISLEPRGLVALPHELESERLYSVALLEGLPRTHRLELYSSERLEGLYGLCYSCYSASPVALGLASPSERTHRTHRCYSPHEASNALALYSGLYPRLELELYSLYSGLLESERSERPHEILEASPLYSGLYGLNGLLECYSALAASPTYRSERGLASNTHRPHETYRTYRLEGLNASNALAPHELEVALALATYRTHRLYSLYSALAPRGLNLETHRSERSERGLLEMETALAILETHRARGLYSMETALAALATHRALAALATHRCYSCYSGLNLESERGLASPLYSLELEALACYSGLYGLGLYALAALAASPILEILEILEGLYHISLECYSILELEHISGLMETTHRPRVALSERASPARGVALTHRGLNCYSCYSTHRSERSERTYRALAASNARGARGPRCYSPHESERSERLEGLVALASPGLTHRTYRVALPRLYSGLPHESERASPASPLYSPHETHRPHEHISSERASPLECYSGLNALAGLNGLYVALALALEGLNTHRMETLYSGLNGLPHELEILEASNLEVALLYSHISLYSPRLYSILETHRGLGLGLNLEGLALAVALILEALAASPPHESERGLYLELEGLLYSCYSCYSGLNGLYGLNGLGLNGLVALCYSPHEALAGLGLGLYTRPLYSLEILESERLYSTHRARGALAALALEGLYVAL

SEQ ID NO: 12 - protein, 3342 aa, artificial sequence
  Synthesized
METLYSTRPVALTHRPHEILESERLELEPHELEPHESERSERALATYRSERARGGLYVALPHEARGARGSERHISTHRGLASPASPILEILEILEALATHRLYSASNGLYLYSVALARGGLYMETASNLETHRVALPHEGLYGLYTHRVALTHRALAPHELEGLYILEPRTYRALAGLNPRPRLEGLYARGLEARGPHELYSLYSPRGLNSERLETHRLYSTRPSERASPILETRPASNALATHRLYSTYRALAASNSERCYSCYSGLNASNILEASPGLNSERPHEPRGLYPHEHISGLYSERGLMETTRPASNPRASNTHRASPLESERGLASPCYSLETYRLEASNVALTRPILEPRALAPRLYSPRLYSASNALATHRVALLEILETRPILETYRGLYGLYGLYPHEGLNTHRGLYTHRSERSERLEHISVALTYRASPGLYLYSPHELEALAARGVALGLARGVALILEVALVALSERMETASNTYRARGVALGLYALALEGLYPHELEALALEPRGLYASNPRGLALAPRGLYASNMETGLYLEPHEASPGLNGLNLEALALEGLNTRPVALGLNLYSASNILEALAALAPHEGLYGLYASNPRLYSSERVALTHRLEPHEGLYGLSERALAGLYALAALASERVALSERLEHISLELESERPRGLYSERHISSERLEPHETHRARGALAILELEGLNSERGLYSERPHEASNALAPRTRPALAVALTHRSERLETYRGLALAARGASNARGTHRLEASNLEALALYSLETHRGLYCYSSERARGGLASNGLTHRGLILEILELYSCYSLEARGASNLYSASPPRGLNGLILELELEASNGLALAPHEVALVALPRTYRGLYTHRPRLESERVALASNPHEGLYPRTHRVALASPGLYASPPHELETHRASPMETPRASPILELELEGLLEGLYGLNPHELYSLYSTHRGLNILELEVALGLYVALASNLYSASPGLGLYTHRALAPHELEVALTYRGLYALAPRGLYPHESERLYSASPASNASNSERILEILETHRARGLYSGLPHEGLNGLGLYLELYSILEPHEPHEPRGLYVALSERGLPHEGLYLYSGLSERILELEPHEHISTYRTHRASPTRPVALASPASPGLNARGPRGLASNTYRARGGLALALEGLYASPVALVALGLYASPTYRASNPHEILECYSPRALALEGLPHETHRLYSLYSPHESERGLTRPGLYASNASNALAPHEPHETYRTYRPHEGLHISARGSERSERLYSLEPRTRPPRGLTRPMETGLYVALMETHISGLYTYRGLILEGLPHEVALPHEGLYLEPRLEGLARGARGASPASNTYRTHRLYSALAGLGLILELESERARGSERILEVALLYSARGTRPALAASNPHEALALYSTYRGLYASNPRASNGLTHRGLNASNASNSERTHRSERTRPPRVALPHELYSSERTHRGLGLNLYSTYRLETHRLEASNTHRGLSERTHRARGILEMETTHRLYSLEARGALAGLNGLNCYSARGPHETRPTHRSERPHEPHEPRLYSVALLEGLMETTHRGLYASNILEASPGLALAGLTRPGLTRPLYSALAGLYPHEHISARGTRPASNASNTYRMETMETASPTRPLYSASNGLNPHEASNASPTYRTHRSERLYSLYSGLSERCYSVALGLYLEGLALAALAALALYSGLALAALAALALYSGLALAALAALALYSGLALAALAALALYSGLALAALAALALYSGLLYSASNLYSVALCYSLYSGLPHESERHISLEGLYLYSGLASPPHETHRSERLESERLEVALLETYRSERARGLYSPHEPRSERGLYTHRPHEGLGLNVALSERGLNLEVALLYSGLVALVALSERLETHRGLALACYSVALALAGLGLYALAASPPRASPCYSTYRASPTHRARGTHRSERALALESERALALYSSERCYSGLSERASNSERPRPHEPRVALHISPRGLYTHRALAGLCYSCYSTHRLYSGLGLYLEGLARGLYSLECYSMETALAALALELYSHISGLNPRGLNGLPHEPRTHRTYRVALGLPRTHRASNASPGLILECYSGLALAPHEARGLYSASPPRLYSGLTYRALAASNGLNPHEMETTRPGLTYRSERTHRASNTYRGLYGLNALAPRLESERLELEVALSERTYRTHRLYSSERTYRLESERMETVALGLYSERCYSCYSTHRSERALASERPRTHRVALCYSPHELELYSGLARGLEGLNLELYSHISLESERLELETHRTHRLESERASNARGVALCYSSERGLNTYRALAALATYRGLYGLLYSLYSSERARGLESERASNLEILELYSLEALAGLNLYSVALPRTHRALAASPLEGLASPVALLEPRLEALAGLASPILETHRASNILELESERLYSCYSCYSGLSERALASERGLASPCYSMETALALYSGLLEPRGLHISTHRVALLYSLECYSASPASNGLNASPTHRLYSASNSERLYSPHEGLASPCYSCYSGLNGLLYSTHRALAMETASPVALPHEVALCYSTHRTYRPHEMETPRALAALAGLNLEPRGLLEPRASPVALGLLEPRTHRASNLYSASPVALCYSASPPRGLYASNTHRLYSVALMETASPLYSTYRTHRPHEGLLESERARGARGTHRHISLEPRGLVALPHELESERLYSVALLEGLPRTHRLELYSSERLEGLYGLCYSCYSASPVALGLASPSERTHRTHRCYSPHEASNALALYSGLYPRLELELYSLYSGLLESERSERPHEILEASPLYSGLYGLNGLLECYSALAASPTYRSERGLASNTHRPHETYRTYRLEGLNASNALAPHELEVALALATYRTHRLYSLYSALAPRGLNLETHRSERSERGLLEMETALAILETHRARGLYSMETALAALATHRALAALATHRCYSCYSGLNLESERGLASPLYSLELEALACYSGLYGLGLYALAALAASPILEILEILEGLYHISLECYSILELEHISGLMETTHRPRVALSERASPARGVALTHRGLNCYSCYSTHRSERSERTYRALAASNARGARGPRCYSPHESERSERLEGLVALASPGLTHRTYRVALPRLYSGLPHESERASPASPLYSPHETHRPHEHISSERASPLECYSGLNALAGLNGLYVALALALEGLNTHRMETLYSGLNGLPHELEILEASNLEVALLYSHISLYSPRLYSILETHRGLGLGLNLEGLALAVALILEALAASPPHESERGLYLELEGLLYSCYSCYSGLNGLYGLNGLGLNGLVALCYSPHEALAGLGLGLYTRPLYSLEILESERLYSTHRARGALAALALEGLYVAL

SEQ ID NO: 13 - DNA, 4025 bp, artificial sequence
  Synthesized
ttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatgggcgtgcacgaatgtcctgcctggctgtggctgctgctgagcctgctgtctctgcctctgggactgcctgtgctgggagcccctcctagactgatctgcgacagccgggtgctggaaagatacctgctggaagccaaagaggccgagaacatcaccaccggctgcgccgagcactgcagcctgaacgagaatatcaccgtgcccgacaccaaagtgaacttctacgcctggaagcggatggaagtgggccagcaggctgtggaagtgtggcagggactggccctgctgagcgaagctgtgctgagaggacaggctctgctcgtgaacagcagccagccttgggagcctctgcagctgcacgtggacaaggccgtgtctggcctgagaagcctgaccacactgctgagagccctgggggcccagaaagaggccatctctccacctgatgccgcctctgccgcccctctgagaaccatcaccgccgacaccttcagaaagctgttccgggtgtacagcaacttcctgcggggcaagctgaagctgtacacaggcgaggcctgccggaccggcgatagataacccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 14 - DNA, 4016 bp, artificial sequence
  Synthesized
ttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatgaagaaaacccagacctggatcctgacctgcatctacctgcagctgctgctgttcaaccccctcgtgaaaaccgagggcatctgccggaacagagtgaccaacaacgtgaaggacgtgaccaagctggtggccaacctgcccaaggactacatgatcaccctgaaatacgtgcccggcatggacgtgctgcccagccactgttggatcagcgagatggtggtgcagctgagcgacagcctgaccgacctgctggacaagttcagcaacatcagcgagggcctgagcaactacagcatcatcgataagctcgtgaacatcgtggacgacctggtggaatgcgtgaaagagaacagctccaaggacctgaagaagtccttcaagagccccgagcccagactgttcacccccgaggaattcttccggatcttcaaccggtccatcgacgccttcaaggacttcgtggtggccagcgagacaagcgactgcgtggtgtctagcaccctgtcccccgagaaggacagcagagtgtccgtgacaaagcccttcatgctgccccctgtggccgcctaacccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 15 - DNA, 4829 bp, Artificial Sequence
  Synthesized
ttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatgcagcgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagctgacataacccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 16 - DNA, 3902 bp, artificial sequence
  Synthesized
ttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatgagcagactgcccgtgctcctgctgctgcagctgctcgtgcggcctggactgcaggctcctatgacccagaccacccccctgaaaaccagctgggtcaactgcagcaacatgatcgacgagatcatcacccacctgaagcagccccccctgcccctgctggacttcaacaacctgaacggcgaggaccaggacatcctgatggaaaacaacctgcggaggcccaacctggaagccttcaacagagccgtgaagtccctgcagaacgccagcgccatcgagagcatcctgaagaacctgctgccctgcctgcctctggccacagccgctcctacaagacaccccatccacatcaaggacggcgactggaacgagttccggcggaagctgaccttctacctgaaaacactggaaaacgcccaggcccagcagaccacactgagcctggccatcttctaacccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 17 - DNA, 4505 bp, artificial sequence
  Synthesized
ttgggtagggaaggaagatttatgaaatatttaaaaaattattcttccttcgctttgtttttagacataatgttaaatttattttgaaatttaaagcaacataaaagaacatgtgatttttctacttattgaaagagagaaaggaaaaaaatatgaaacagggatggaaagaatcctatgcctggtgaaggtcaagggttctcataacctacagagaatttggggtcagcctgtcctattgtatattatggcaaagataatcatcatctcatttgggtccattttcctctccatctctgcttaactgaagatcccatgagatatactcacactgaatctaaatagcctatctcagggcttgaatcacatgtgggccacagcaggaatgggaacatggaatttctaagtcctatcttacttgttattgttgctatgtctttttcttagtttgcatctgaggcaacatcagctttttcagacagaatggctttggaatagtaaaaaagacacagaagccctaaaatatgtatgtatgtatatgtgtgtgtgcatgcgtgagtacttgtgtgtaaatttttcattatctataggtaaaagcacacttggaattagcaatagatgcaatttgggacttaactctttcagtatgtcttatttctaagcaaagtatttagtttggttagtaattactaaacactgagaactaaattgcaaacaccaagaactaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacaatggaactgaccgagctgctgctggtcgtgatgctgctgctgaccgccagactgaccctgtctagccctgcccctcctgcctgcgatctgagagtgctgagcaagctgctgcgggacagccacgtgctgcacagcagactgagccagtgccctgaggtgcaccctctgcctacacctgtgctgctgcctgccgtggatttcagcctgggcgagtggaaaacccagatggaagagacaaaggcccaggacatcctgggagccgtgaccctgctgctggaaggcgtgatggctgccagaggacagctgggccctacctgtctgtcctctctgctgggccagctgtctggacaagtgcggctgctgctgggagccctgcagtctctgctgggaacacagctgcctccccagggcagaaccaccgcccacaaggaccccaacgccatcttcctgagcttccagcatctgctgagaggcaaagtgcggttcctgatgctcgtgggcggcagcacactgtgcgtgcggagagcacctcctaccacagccgtgcctagcagaaccagcctggtgctgaccctgaacgagctgcccaacagaacctccggcctgctggaaacaaacttcaccgccagcgccaggaccacaggctctggactgctgaagtggcagcagggcttccgggccaagattcctggcctgctgaaccagaccagcagaagcctggaccagatccccggctacctgaaccggatccacgaactgctgaacggcaccagaggcctgttcccaggcccctccagaagaacactgggcgctcccgatatcagcagcggcacctctgataccggcagcctgccccctaatctgcagcctggctacagccctagccctacccaccctccaaccggccagtacaccctgttccctctgccacctaccctgcccacaccagtggtgcagctgcatcctctgctgcccgatcctagcgcccctacccctacaccaacaagccccctgctgaataccagctacacccacagccagaacctgagccaggaaggctaacccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaccccgggataattcctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctattttgtagatgagaaaatgaagataaacatcaaagcatagattaagtaattttccaaagggtcaaaattcaaaattgaaaccaaagtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 18 - DNA, 1386 bp, Artificial Sequence
  Synthesized
atgcagcgcgtgaacatgattatggccgagagccctggcctgatcaccatctgcctgctgggctacctgctgagcgccgagtgcaccgtgtttctggaccacgagaacgccaacaagatcctgaaccggcccaagcggtacaacagcggcaagctggaagagttcgtgcagggcaacctggaacgcgagtgcatggaagagaagtgcagcttcgaagaggccagagaggtgttcgagaacaccgagcggaccaccgagttctggaagcagtacgtggacggcgaccagtgcgagagcaacccctgtctgaatggcggcagctgcaaggacgacatcaacagctacgagtgctggtgccccttcggcttcgagggcaagaactgcgagctggacgtgacctgcaacatcaagaacggcagatgcgagcagttctgcaagaacagcgccgacaacaaggtcgtgtgctcctgcaccgagggctacagactggccgagaaccagaagtcctgcgagcccgccgtgcctttcccatgtggaagagtgtccgtgtcccagaccagcaagctgaccagagccgagacagtgttccccgacgtggactacgtgaacagcaccgaggccgagacaatcctggacaacatcacccagagcacccagtccttcaacgacttcaccagagtcgtgggcggcgaggatgccaagcctggacagttcccgtggcaggtggtgctgaacggaaaggtggacgccttttgcggcggcagcatcgtgaacgagaagtggatcgtgacagccgcccactgcgtggaaaccggcgtgaagattacagtggtggccggcgagcacaacatcgaggaaaccgagcacacagagcagaaacggaacgtgatcagaatcatcccccaccacaactacaacgccgccatcaacaagtacaaccacgatatcgccctgctggaactggacgagcccctggtgctgaatagctacgtgacccccatctgtatcgccgacaaagagtacaccaacatctttctgaagttcggcagcggctacgtgtccggctggggcagagtgtttcacaagggcagatccgctctggtgctgcagtacctgagagtgcctctggtggaccgggccacctgtctgagaagcaccaagttcaccatctacaacaacatgttctgcgccggctttcacgagggcggcagagatagctgtcagggcgattctggcggccctcacgtgacagaggtggaaggcaccagctttctgaccggcatcatcagctggggcgaggaatgcgccatgaaggggaagtacggcatctacaccaaggtgtccagatacgtgaactggatcaaagaaaagaccaagctgacataa

SEQ ID NO: 19 - DNA, 582 bp, Artificial Sequence
  Synthesized
atgggcgtgcacgaatgtcctgcctggctgtggctgctgctgagcctgctgtctctgcctctgggactgcctgtgctgggagcccctcctagactgatctgcgacagccgggtgctggaaagatacctgctggaagccaaagaggccgagaacatcaccaccggctgcgccgagcactgcagcctgaacgagaatatcaccgtgcccgacaccaaagtgaacttctacgcctggaagcggatggaagtgggccagcaggctgtggaagtgtggcagggactggccctgctgagcgaagctgtgctgagaggacaggctctgctcgtgaacagcagccagccttgggagcctctgcagctgcacgtggacaaggccgtgtctggcctgagaagcctgaccacactgctgagagccctgggggcccagaaagaggccatctctccacctgatgccgcctctgccgcccctctgagaaccatcaccgccgacaccttcagaaagctgttccgggtgtacagcaacttcctgcggggcaagctgaagctgtacacaggcgaggcctgccggaccggcgatagataa

SEQ ID NO: 20 - DNA, 459 bp, Artificial Sequence
  Synthesized
atgagcagactgcccgtgctcctgctgctgcagctgctcgtgcggcctggactgcaggctcctatgacccagaccacccccctgaaaaccagctgggtcaactgcagcaacatgatcgacgagatcatcacccacctgaagcagccccccctgcccctgctggacttcaacaacctgaacggcgaggaccaggacatcctgatggaaaacaacctgcggaggcccaacctggaagccttcaacagagccgtgaagtccctgcagaacgccagcgccatcgagagcatcctgaagaacctgctgccctgcctgcctctggccacagccgctcctacaagacaccccatccacatcaaggacggcgactggaacgagttccggcggaagctgaccttctacctgaaaacactggaaaacgcccaggcccagcagaccacactgagcctggccatcttctaa

SEQ ID NO: 21 - DNA, 573 bp, Artificial Sequence
  Synthesized
atgaagaaaacccagacctggatcctgacctgcatctacctgcagctgctgctgttcaaccccctcgtgaaaaccgagggcatctgccggaacagagtgaccaacaacgtgaaggacgtgaccaagctggtggccaacctgcccaaggactacatgatcaccctgaaatacgtgcccggcatggacgtgctgcccagccactgttggatcagcgagatggtggtgcagctgagcgacagcctgaccgacctgctggacaagttcagcaacatcagcgagggcctgagcaactacagcatcatcgataagctcgtgaacatcgtggacgacctggtggaatgcgtgaaagagaacagctccaaggacctgaagaagtccttcaagagccccgagcccagactgttcacccccgaggaattcttccggatcttcaaccggtccatcgacgccttcaaggacttcgtggtggccagcgagacaagcgactgcgtggtgtctagcaccctgtcccccgagaaggacagcagagtgtccgtgacaaagcccttcatgctgccccctgtggccgcctaa

SEQ ID NO: 22 - DNA, 1062 bp, artificial sequence
  aSynthesized
atggaactgaccgagctgctgctggtcgtgatgctgctgctgaccgccagactgaccctgtctagccctgcccctcctgcctgcgatctgagagtgctgagcaagctgctgcgggacagccacgtgctgcacagcagactgagccagtgccctgaggtgcaccctctgcctacacctgtgctgctgcctgccgtggatttcagcctgggcgagtggaaaacccagatggaagagacaaaggcccaggacatcctgggagccgtgaccctgctgctggaaggcgtgatggctgccagaggacagctgggccctacctgtctgtcctctctgctgggccagctgtctggacaagtgcggctgctgctgggagccctgcagtctctgctgggaacacagctgcctccccagggcagaaccaccgcccacaaggaccccaacgccatcttcctgagcttccagcatctgctgagaggcaaagtgcggttcctgatgctcgtgggcggcagcacactgtgcgtgcggagagcacctcctaccacagccgtgcctagcagaaccagcctggtgctgaccctgaacgagctgcccaacagaacctccggcctgctggaaacaaacttcaccgccagcgccaggaccacaggctctggactgctgaagtggcagcagggcttccgggccaagattcctggcctgctgaaccagaccagcagaagcctggaccagatccccggctacctgaaccggatccacgaactgctgaacggcaccagaggcctgttcccaggcccctccagaagaacactgggcgctcccgatatcagcagcggcacctctgataccggcagcctgccccctaatctgcagcctggctacagccctagccctacccaccctccaaccggccagtacaccctgttccctctgccacctaccctgcccacaccagtggtgcagctgcatcctctgctgcccgatcctagcgcccctacccctacaccaacaagccccctgctgaataccagctacacccacagccagaacctgagccaggaaggctaa

(19) United States
(12) Patent Application Publication
(10) Pub. No.: US 2016/0207978 A1
(43) Pub. Date: Jul. 21, 2016

(54) METHODS AND COMPOSITIONS RELATED TO LARGE SCALE PRODUCTION OF PROTEINS

(71) Applicant: CELL MACHINES INC., Houston, TX (US)

(72) Inventor: James Kelly, Houston, TX (US)

(21) Appl. No.: 14/889,513

(22) PCT Filed: May 6, 2014
(86) PCT No.: PCT/US14/36947

§ 371 Date: Nov. 6, 2015

Related U.S. Application Data
(60) Provisional application No. 61/819,912, filed on May 6, 2013.
(60) Provisional application No. 61/954,692, filed on Mar. 18, 2014.

(51) Int. Cl. C07K 14/76; C07K 16/46; C07K 14/755; C07K 16/00; C12N 9/64; C12N 9/18

(52) U.S. Cl. CPC C07K 14/76; C12N 9/644; C12N 9/18; C07K 14/755; C07K 16/00; C07K 16/468; C12Y 304/21022; C12Y 301/01008; C07K 2319/31; C07K 2317/622; C07K 2317/31; C07K 2317/94

(57) ABSTRACT

Disclosed herein are methods and compositions related to synthetic fusion proteins and engineered cells.

### CROSS-REFERENCE TO RELATED APPLICATIONS

This application claims benefit of U.S. Provisional Application No. 61/819,912, filed May 6, 2013, and to U.S. Provisional Application No. 61/954,692, filed Mar. 18, 2014, both of which are hereby incorporated by reference in their entireties.

### BACKGROUND

Therapeutic proteins are widely employed in treating various acquired and genetic diseases such as cancer and enzyme deficiencies (Wurm et al. Nat. Biotechnol. 22, 1393-1398). There are three major problems impeding even more widespread use. First, they are difficult and expensive to produce. Second, they are often modified by glycosylation or sulfation processes which are poorly reproduced in most mammalian cell culture systems. Third, they often have a short half-life in vivo, necessitating frequent injection or infusion. Accordingly, there is a need for more efficient and effective compositions and methods for protein production and use.

### SUMMARY

Disclosed herein are nucleic acids encoding a non-albumin protein (also referred to herein as a protein of interest) operably inserted into an albumin gene locus in a hepatocyte or hepatocyte-derived cell line. Also disclosed are nucleic acids encoding a non-albumin protein (protein of interest) operably inserted into a non-endogenous gene; wherein the non-endogenous gene is in a hepatocyte or hepatocyte-derived cell line. Also provided is a hepatocyte or hepatocyte-derived cell line lacking endogenous albumin coding sequence, comprising a nucleic acid encoding a non-albumin protein (protein of interest) operably inserted into a non-endogenous gene, for example, the albumin gene locus.

Also disclosed is a protein produced by a nucleic acid encoding a non-albumin protein operably inserted into nucleic acid encoding a non-albumin protein, for example, an albumin gene locus, in a hepatocyte or hepatocyte-derived cell line.

Also disclosed herein is a system which is useful for producing a protein from the engineered cells disclosed herein.

Further disclosed herein is a method of producing a non-albumin protein, the method comprising a) culturing the engineered cells disclosed herein; and b) allowing the cell to produce the non-albumin protein.

Disclosed are polypeptides comprising multiple domains, where at least two domains are selected from different members of the albumin superfamily. This is referred to as an SFP, or synthetic fusion protein. The SFP can have one, two, three, four, or more domains. The polypeptide can be fused to a protein of interest (POI), and together this molecule is referred to as the SFP-POI.

Also disclosed are methods of modulating distribution of a protein of interest within a subject, the method comprising administering to the subject the polypeptide described above, wherein the polypeptide modulates the distribution of the peptide of interest within the subject.

Also disclosed is a method of treating a subject with a disease, or preventing said disease in the subject, the method comprising administering to the subject the polypeptide described herein, wherein the protein of interest is able to treat or prevent the disease.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a computer generated model of SFP compared to the actual structure of human albumin. The triangle marks the conserved histidine triad that is responsible for binding to the neonatal Fc receptor.

FIG. 2 shows albumin synthesis in cultures of the C3A cell line.

FIG. 3 shows a diagram of insertion into the albumin locus.

FIGS. 4A-C shows construction of the Factor IX (FIXneo) and Stabile9 (S9neo) targeting plasmids.

FIG. 5 shows BChE and StabileBChE targeting plasmids.

FIG. 6 shows G418 resistant clones analyzed for insertion into the albumin locus via PCR.

FIG. 7 shows clones analyzed for presence of Factor IX in the supernate via ELISA.

FIG. 8 shows clones analyzed for Factor IX enzyme activity.

FIG. 9 shows clones analyzed for mRNA via QPCR.

FIG. 10 shows codon optimization increases production of Factor IX mRNA.

FIG. 11 shows the structure of the Factor IX minigene construct.

### DETAILED DESCRIPTION

The materials, compositions, and methods described herein can be understood more readily by reference to the following detailed descriptions of specific aspects of the disclosed subject matter and the Examples and Figure included herein.

Before the present materials, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “an enzyme” includes mixtures of two or more such enzymes; reference to “the probiotic” includes mixtures of two or more such probiotics, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. “About” can mean within 5% of the stated value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “5” is disclosed, then “about 5” is also disclosed.

### Non-Endogenous Genes in Hepatocyte Cells

Disclosed herein are nucleic acids encoding a non-albumin protein (also referred to herein as a protein of interest) operably inserted into an albumin gene locus in a hepatocyte or hepatocyte-derived cell line. It is noted that the entire, or part of, the endogenous gene can be replaced by the nucleic acid encoding the non-albumin protein. Also disclosed are nucleic acids encoding a non-albumin protein (protein of interest) operably inserted into a non-endogenous gene selected from the group consisting of: alpha-1-microglobulin/bikunin precursor; alpha-2-HS-glycoprotein; alphafetoprotein; apolipoprotein A2; apolipoprotein C1; apolipoprotein H; fibrinogen gamma chain; serpin peptidase inhibitor, clade A, member 1; serpin peptidase inhibitor, clade A, member 3; serpin peptidase inhibitor, clade A, member 7; serpin peptidase inhibitor, clade C, member 1; and transferrin; wherein the non-endogenous gene is in a hepatocyte or hepatocyte-derived cell line. Also disclosed herein are cells comprising the nucleic acids encoding a non-albumin protein operably inserted into an albumin gene or other non-endogenous gene locus.

By “non-endogenous gene locus” is meant a gene other than that of the endogenous gene of the protein of interest. Non-limiting examples of genes which can be used as insertion sites are found in Table 1:

TABLE 1
Non-Endogenous Genes

| Target Gene Name | Gene Symbol | Common Name |
|---|---|---|
| Albumin | ALB | Albumin |
| Alpha-1-microglobulin/ | AMBP | Alpha-1-microglobulin |
| bikunin precursor | | |
| Alpha-2-HS-glycoprotein | AHSG | Fetuin |
| Alphafetoprotein | AFP | Alphafetoproteins |
| Apolipoprotein A2 | APOA2 | Apolipoprotein A2 |
| Apolipoprotein C1 | APOC1 | Apolipoprotein C1 |
| Apolipoprotein H | APOH | Beta-2-glycoprotein |
| Fibrinogen gamma chain | FBG | Fibrinogen gamma |
| Serpin peptidase inhibitor, | SERPINA1 | Alpha-1-antitrypsin |
| clade A, member 1 | | |
| Serpin peptidase inhibitor, | SERPINA3 | Anticyhmotrypsin |
| clade A, member 3 | | |
| Serpin peptidase inhibitor, | SERPINA7 | Thyroxine binding |
| clade A, member 7 | | globulin |
| Serpin peptidase inhibitor, | SERPINC1 | Antithrombin III |
| clade C, member 1 | | |
| Transferrin | TF | Transferrin |

By “hepatocyte” is meant a cell of the main tissue of the liver. By “hepatocyte-derived cell line” is meant functional hepatocytes derived from cells, such as human stem cells. For example, the cell can be an immortal liver cell. Examples of hepatocytes useful with the systems and methods disclosed herein include, but are not limited to, those found in Table 2, below:

TABLE 2
Cell Lines

| Human Liver Cell Lines | Type |
|---|---|
| Hep3B2.1-7 | ATCC HB-8064 |
| HepG2 | ATCC HB-8065 |
| C3A (HepG2/C3A) | CRL-10741 |
| HuH-7 | JCRB0403 |
| HuH-6 | JCRB0401 |

ATCC—American Type Culture Collection
JCRB—Japanese Collection of Bioresources Cell Bank

The nucleic acids referred to herein encode a non-albumin protein. “Non-albumin protein” refers to any protein which is not a native albumin protein. These proteins are also referred to herein as “proteins of interest” or “peptides of interest.” The protein of interest can have one or more therapeutic and/or biological activities. Therapeutic proteins include but are not limited to, proteins, polypeptides, peptides, antibodies, and biologics. (The terms peptides, proteins, and polypeptides can be used interchangeably herein.) Proteins of interest are further defined herein.

Specifically, the “protein of interest,” or “non-albumin protein,” is a protein that has an activity, e.g. biological or industrial. In a preferred embodiment, the activity is a biological activity that is useful for treating, preventing or ameliorating a disease, or for the production of products useful therein. A non-inclusive list of biological activities that may be possessed by a protein of interest includes, enhancing the immune response, promoting angiogenesis, inhibiting angiogenesis, regulating hematopoietic functions, stimulating nerve growth, enhancing an immune response, inhibiting an immune response, affecting cell metabolism, or any one or more of the biological functions.

Examples of non-albumin proteins, or proteins of interest, include those found in Table 3. This list is intended to be non-limiting, as one of skill in the art can readily envision other proteins of interest useful with the invention.

TABLE 3
Proteins of Interest/Non-Albumin Proteins

| | |
|---|---|
| Factor IX | gonadotropin releasing hormone |
| Factor VIII | keratinocyte growth factor |
| Erythropoietin | platelet derived growth factor |
| Thrombopoietin | collagenase |
| Stem Cell Factor (KIT ligand) | deoxyribonuclease (Dnase) |
| Interleukin 3 | hyaluronidase |
| Interleukin 6 | papain |
| Insulin | L-asparaginase |
| Flt3 | hirudin |
| phenylalanine hyroxylase | streptokinase |
| pramlintide | bevacizumab |
| growth hormone (somatotropin) | cetuximab |
| mecasermin | panitumumab |
| protein C | alemtuzumab |
| Factor VIIa | rituximab |
| beta-glucocerebrosidase | trastuzumab |
| aglucosidase-alpha | abatacept |
| laronidase | anakinra |
| idursuphase | adalimumab |
| galsulfase | etanercept |
| agalsidase-beta | infliximab |
| lactase | alefacept |
| lipase | efalizumab |
| amylase | natalizumab |
| adenosine deaminase | eculizumab |
| darbepoetin | antithymocyte globulin |
| granulocyte colony stimulating | basiliximab |
| factor | |
| granulocyte macrophage colony | daclizumab |
| stimulating factor | |
| interleukin 11 | muromonoab-CD3 |
| follicle stimulating hormone | omalizumab |
| human chorionic gonadotropin | palivizumab |
| lutropin-alpha | enfuviride |
| alpha-interferon | abciximab |
| interferon-beta | pegvisomant |
| interferon-gamma | ranibizumab |
| interleukin 2 | denileukin difitox |
| tissue plasminogen activator | ibritumomab |
| urokinase | gentuzomab |
| exenatide | tositumomab |
| octreotide | glucagon |
| bone morphogenic peptide 2 | growth hormone releasing hormone |
| bone morphogenic protein 7 | secretin |
| thyroid stimulating hormone | nofetumomab |
| capromab pendetide | apcitide |
| satumomab pendetide | imcimomab |
| arcitumomab | |

In an example, the non-albumin protein can be used in red blood cell production. A variety of proteins are required to direct hematopoietic stem cells to differentiate into the various cells of the hematopoietic system, such as erythrocytes (red cells) (Migliaccio A. R. Whitsett, C., Papayannopoulou, T., & Sadelain, M. (2012). The Potential of Stem Cells as an In Vitro Source of Red Blood Cells for Transfusion. Stem Cell, 10(2), 115-119).

These proteins can be applied sequentially or in combination. These proteins are required in large quantities to direct red cell production in vitro for use as a therapeutic. By inserting synthetic genes coding for these proteins into one or more of the non-endogenous genes listed above, the liver cells can be used to generate these quantities. Examples of proteins useful in red blood cell production include, but are not limited to, erythropoietin, thrombopoietin, stem cell factor (KIT ligand), interleukin 3, interleukin 6, insulin, and flt3.

It is also contemplated herein that more than one nucleic acid can be inserted into different genes, specifically including the same nucleic acid inserted into two or more different genes. The protein of interest for the second or more nucleic acid can be selected from Table 3. Alternatively, two or more nucleic acids which encode the same protein of interest can be inserted into two different genes. And lastly, it is envisioned that two nucleic acids which encode different proteins of interest can be inserted into two or more different genes. This is not limited to two different genes or two different nucleic acids, but can be extended to three, four, five, six, seven, eight, nine, ten, or more different nucleic acids encoding different proteins of interest, and they can be inserted into one, two, three, four, five, six, seven, eight, nine, ten or more different non-endogenous insertion genes.

It is also noted that nucleic acids encoding any of the proteins of interest present in Table 3 can be inserted into any of the non-endogenous genes in Table 1, and these insertions can be used in any of the cells found in Table 2. For example, in Table 4, below, any of the proteins of interest in the left column can be used with any of the non-endogenous genes of the center column, and they can be inserted into any of the cells in the right column. Therefore, every combination of proteins of interest, genes, and cells listed below in Table 4 is herein contemplated.

TABLE 4
Proteins of Interest, Non-Endogenous Genes for
Insertion, and Cells Useful in Combination

| Proteins of | | |
|---|---|---|
| Interest/Non- | | |
| Albumin Proteins | Non-Endogenous Gene | Liver Cell Type |
| Factor IX | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Factor VIII | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Erythropoietin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Thrombopoietin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Stem Cell Factor | alpha-1-microglobulin/bikunin precursor; | Hep3B2.1-7, HepG2, |
| (KIT ligand) | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Interleukin 3 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Interleukin 6 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Insulin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Flt3 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| phenylalanine | alpha-1-microglobulin/bikunin precursor; | Hep3B2.1-7, HepG2, |
| hyroxylase | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| pramlintide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| growth hormone | alpha-1-microglobulin/bikunin precursor; | Hep3B2.1-7, HepG2, |
| (somatotropin) | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| mecasermin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| protein C | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| Factor VIIa | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| beta- | alpha-1-microglobulin/bikunin precursor; | Hep3B2.1-7, HepG2, |
| glucocerebrosidase | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| aglucosidase-alpha | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| laronidase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| idursuphase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| galsulfase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferring | |
| agalsidase-beta | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| lactase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| lipase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| amylase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| adenosine deaminase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| darbepoetin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| granulocyte colony | alpha-1-microglobulin/bikunin precursor; | Hep3B2.1-7, HepG2, |
| stimulating factor | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| granulocyte | alpha-1-microglobulin/bikunin precursor; | Hep3B2.1-7, HepG2, |
| macrophage colony | alpha-2-HS-glycoprotein; alphafetoprotein; | C3A (HepG2/C3A), |
| stimulating factor | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| interleukin 11 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| follicle | alpha-1-microglobulin/bikunin precursor; | Hep3B2.1-7, HepG2, |
| stimulating | alpha-2-HS-glycoprotein; alphafetoprotein; | C3A (HepG2/C3A), |
| hormone | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| human chorionic | alpha-1-microglobulin/bikunin precursor; | Hep3B2.1-7, HepG2, |
| gonadotropin | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| lutropin-alpha | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| alpha-interferon | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| interferon-beta | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| interferon-gamma | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| interleukin 2 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| tissue plasminogen | alpha-1-microglobulin/bikunin precursor; | Hep3B2.1-7, HepG2, |
| activator | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| urokinase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| exenatide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| octreotide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| bone morphogenic | alpha-1-microglobulin/bikunin precursor; | Hep3B2.1-7, HepG2, |
| peptide 2 | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| bone morphogenic | alpha-1-microglobulin/bikunin precursor; | Hep3B2.1-7, HepG2, |
| protein 7 | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| thyroid stimulating | alpha-1-microglobulin/bikunin precursor; | Hep3B2.1-7, HepG2, |
| hormone | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| capromab pendetide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| satumomab pendetide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| arcitumomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| gonadotropin | alpha-1-microglobulin/bikunin precursor; | Hep3B2.1-7, HepG2, |
| releasing | alpha-2-HS-glycoprotein; alphafetoprotein; | C3A (HepG2/C3A), |
| hormone | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| keratinocyte | alpha-1-microglobulin/bikunin precursor; | Hep3B2.1-7, HepG2, |
| growth factor | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| platelet derived | alpha-1-microglobulin/bikunin precursor; | Hep3B2.1-7, HepG2, |
| growth factor | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| collagenase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| deoxyribonuclease | alpha-1-microglobulin/bikunin precursor; | Hep3B2.1-7, HepG2, |
| (Dnase) | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| hyaluronidase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| papain | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| L-asparaginase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| hirudin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| streptokinase | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| bevacizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| cetuximab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| panitumumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| alemtuzumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| rituximab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| trastuzumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| abatacept | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| anakinra | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| adalimumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| etanercept | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| infliximab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| alefacept | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| efalizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| natalizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| eculizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| antithymocyte | alpha-1-microglobulin/bikunin precursor; | Hep3B2.1-7, HepG2, |
| globulin | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| basiliximab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| daclizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| muromonoab-CD3 | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| omalizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| palivizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| enfuviride | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| abciximab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| pegvisomant | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| ranibizumab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| denileukin difitox | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| ibritumomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| gentuzomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| tositumomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| glucagon | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| growth hormone | alpha-1-microglobulin/bikunin precursor; | Hep3B2.1-7, HepG2, |
| releasing hormone | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| secretin | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| nofetumomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| apcitide | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |
| imcimomab | alpha-1-microglobulin/bikunin precursor; | Hep3B2.1-7, HepG2, |
| | alpha-2-HS-glycoprotein; alphafetoprotein; | C3A (HepG2/C3A), |
| | apolipoprotein A2; apolipoprotein C1; | HuH-7, and HuH-6 |
| | apolipoprotein H; fibrinogen gamma chain; | |
| | serpin peptidase inhibitor, clade A, member 1; | |
| | serpin peptidase inhibitor, clade A, member 3; | |
| | serpin peptidase inhibitor, clade A, member 7; | |
| | serpin peptidase inhibitor, clade C, member 1; | |
| | albumin; and transferrin | |

Disclosed in Table 5 are specific examples of proteins of interest, the target gene into which nucleic acid encoding the protein of interest can be inserted, and cells in which the expression system can be used.

TABLE 5
Specific Proteins of Interest, Non-Endogenous Genes
for Insertion, and Cells Useful in Combination

| Target Gene Name | Protein of Interest | Liver Cell Type |
|---|---|---|
| Albumin | Factor IX | Hep3B2.1-7, HepG2, |
| | | C3A (HepG2/C3A), HuH-7, and |
| | | HuH-6 |
| Albumin | Factor VIII | Hep3B2.1-7, HepG2, |
| | | C3A (HepG2/C3A), HuH-7, and |
| | | HuH-6 Hep3B2.1-7, HepG2, |
| | | C3A (HepG2/C3A), HuH-7, and |
| | | HuH-6 |
| Albumin | Erythropoietin | Hep3B2.1-7, HepG2, |
| | | C3A (HepG2/C3A), HuH-7, and |
| | | HuH-6 |
| Albumin | Thrombopoietin | Hep3B2.1-7, HepG2, |
| | | C3A (HepG2/C3A), HuH-7, and |
| | | HuH-6 |
| Albumin | Stem Cell Factor (KIT | Hep3B2.1-7, HepG2, |
| | ligand) | C3A (HepG2/C3A), HuH-7, and |
| | | HuH-6 |
| Albumin | Interleukin 3 | Hep3B2.1-7, HepG2, |
| | | C3A (HepG2/C3A), HuH-7, and |
| | | HuH-6 |
| Albumin | Interleukin 6 | Hep3B2.1-7, HepG2, |
| | | C3A (HepG2/C3A), HuH-7, and |
| | | HuH-6 |
| Albumin | Granulocyte macrophage | Hep3B2.1-7, HepG2, |
| | colony stimulating factor | C3A (HepG2/C3A), HuH-7, and |
| | | HuH-6 |
| Albumin | Flt3 | Hep3B2.1-7, HepG2, |
| | | C3A (HepG2/C3A), HuH-7, and |
| | | HuH-6 |
| Albumin | phenylalanine | Hep3B2.1-7, HepG2, |
| | hyroxylase | C3A (HepG2/C3A), HuH-7, and |
| | | HuH-6 |
| Alpha-1- | Factor IX | Hep3B2.1-7, HepG2, |
| microglobulin/bikunin precursor | | C3A (HepG2/C3A), HuH-7, and |
| | | HuH-6 |
| Alpha-1- | Factor VIII | Hep3B2.1-7, HepG2, |
| microglobulin/bikunin precursor | | C3A (HepG2/C3A), HuH-7, and |
| | | HuH-6 |
| Alpha-1- | Erythropoietin | Hep3B2.1-7, HepG2, |
| microglobulin/bikunin precursor | | C3A (HepG2/C3A), HuH-7, and |
| | | HuH-6 |
| Alpha-1- | Thrombopoietin | Hep3B2.1-7, HepG2, |
| microglobulin/bikunin precursor | | C3A (HepG2/C3A), HuH-7, and |
| | | HuH-6 |
| Alpha-1- | Stem Cell Factor (KIT | Hep3B2.1-7, HepG2, |
| microglobulin/bikunin precursor | ligand) | C3A (HepG2/C3A), HuH-7, and |
| | | HuH-6 |
| Alpha-1- | Interleukin 3 | Hep3B2.1-7, HepG2, |
| microglobulin/bikunin precursor | | C3A (HepG2/C3A), HuH-7, and |
| | | HuH-6 |
| Alpha-1- | Interleukin 6 | Hep3B2.1-7, HepG2, |
| microglobulin/bikunin precursor | | C3A (HepG2/C3A), HuH-7, and |
| | | HuH-6 |
| Alpha-1- | Granulocyte macrophage | Hep3B2.1-7, HepG2, |
| microglobulin/bikunin precursor | colony stimulating factor | C3A (HepG2/C3A), HuH-7, and |
| | | HuH-6 |
| Alpha-1- | Flt3 | Hep3B2.1-7, HepG2, |
| microglobulin/bikunin precursor | | C3A (HepG2/C3A), HuH-7, and |
| | | HuH-6 |
| Alpha-1- | phenylalanine | Hep3B2.1-7, HepG2, |
| microglobulin/bikunin precursor | hyroxylase | C3A (HepG2/C3A), HuH-7, and |
| | | HuH-6 |
| Alpha-2-HS- | Factor IX | Hep3B2.1-7, HepG2, |
| glycoprotein | | C3A (HepG2/C3A), HuH-7, and |
| | | HuH-6 |
| Alpha-2-HS- | Factor VIII | Hep3B2.1-7, HepG2, |
| glycoprotein | | C3A (HepG2/C3A), HuH-7, and |
| | | HuH-6 |
| Alpha-2-HS- | Erythropoietin | Hep3B2.1-7, HepG2, |
| glycoprotein | | C3A (HepG2/C3A), HuH-7, and |
| | | HuH-6 |
| Alpha-2-HS- | Thrombopoietin | Hep3B2.1-7, HepG2, |
| glycoprotein | | C3A (HepG2/C3A), HuH-7, and |
| | | HuH-6 |
| Alpha-2-HS- | Stem Cell Factor (KIT | Hep3B2.1-7, HepG2, |
| glycoprotein | ligand) | C3A (HepG2/C3A), HuH-7, and |
| | | HuH-6 |
| Alpha-2-HS- | Interleukin 3 | Hep3B2.1-7, HepG2, |
| glycoprotein | | C3A (HepG2/C3A), HuH-7, and |
| | | HuH-6 |
| Alpha-2-HS- | Interleukin 6 | Hep3B2.1-7, HepG2, |
| glycoprotein | | C3A (HepG2/C3A), HuH-7, and |
| | | HuH-6 |
| Alpha-2-HS- | Granulocyte macrophage | Hep3B2.1-7, HepG2, |
| glycoprotein | colony stimulating factor | C3A (HepG2/C3A), HuH-7, and |
| | | HuH-6 |
| Alpha-2-HS- | Flt3 | Hep3B2.1-7, HepG2, |
| glycoprotein | | C3A (HepG2/C3A), HuH-7, and |
| | | HuH-6 |
| Alpha-2-HS- | phenylalanine | Hep3B2.1-7, HepG2, |
| glycoprotein | hyroxylase | C3A (HepG2/C3A), HuH-7, and |
| | | HuH-6 |
| Apolipoprotein A2 | Factor IX | Hep3B2.1-7, HepG2, |
| | | C3A (HepG2/C3A), HuH-7, and |
| | | HuH-6 |
| Apolipoprotein A2 | Factor VIII | Hep3B2.1-7, HepG2, |
| | | C3A (HepG2/C3A), HuH-7, and |
| | | HuH-6 |
| Apolipoprotein A2 | Erythropoietin | Hep3B2.1-7, HepG2, |
| | | C3A (HepG2/C3A), HuH-7, and |
| | | HuH-6 |
| Apolipoprotein A2 | Thrombopoietin | Hep3B2.1-7, HepG2, |
| | | C3A (HepG2/C3A), HuH-7, and |
| | | HuH-6 |
| Apolipoprotein A2 | Stem Cell Factor (KIT | Hep3B2.1-7, HepG2, |
| | ligand) | C3A (HepG2/C3A), HuH-7, and |
| | | HuH-6 |
| Apolipoprotein A2 | Interleukin 3 | Hep3B2.1-7, HepG2, |
| | | C3A (HepG2/C3A), HuH-7, and |
| | | HuH-6 |
| Apolipoprotein A2 | Interleukin 6 | Hep3B2.1-7, HepG2, |
| | | C3A (HepG2/C3A), HuH-7, and |
| | | HuH-6 |
| Apolipoprotein A2 | Granulocyte macrophage | Hep3B2.1-7, HepG2, |
| | colony stimulating factor | C3A (HepG2/C3A), HuH-7, and |
| | | HuH-6 |
| Apolipoprotein A2 | Flt3 | Hep3B2.1-7, HepG2, |
| | | C3A (HepG2/C3A), HuH-7, and |
| | | HuH-6 |
| Apolipoprotein A2 | phenylalanine | Hep3B2.1-7, HepG2, |
| | hyroxylase | C3A (HepG2/C3A), HuH-7, and |
| | | HuH-6 |
| Apolipoprotein C1 | Factor IX | Hep3B2.1-7, HepG2, |
| | | C3A (HepG2/C3A), HuH-7, and |
| | | HuH-6 |
| Apolipoprotein C1 | Factor VIII | Hep3B2.1-7, HepG2, |
| | | C3A (HepG2/C3A), HuH-7, and |
| | | HuH-6 |
| Apolipoprotein C1 | Erythropoietin | Hep3B2.1-7, HepG2, |
| | | C3A (HepG2/C3A), HuH-7, and |
| | | HuH-6 |
| Apolipoprotein C1 | Thrombopoietin | Hep3B2.1-7, HepG2, |
| | | C3A (HepG2/C3A), HuH-7, and |
| | | HuH-6 |
| Apolipoprotein C1 | Stem Cell Factor (KIT | Hep3B2.1-7, HepG2, |
| | ligand) | C3A (HepG2/C3A), HuH-7, and |
| | | HuH-6 |
| Apolipoprotein C1 | Interleukin 3 | Hep3B2.1-7, HepG2, |
| | | C3A (HepG2/C3A), HuH-7, and |
| | | HuH-6 |
| Apolipoprotein C1 | Interleukin 6 | Hep3B2.1-7, HepG2, |
| | | C3A (HepG2/C3A), HuH-7, and |
| | | HuH-6 |
| Apolipoprotein C1 | Granulocyte macrophage | Hep3B2.1-7, HepG2, |
| | colony stimulating factor | C3A (HepG2/C3A), HuH-7, and |
| | | HuH-6 |
| Apolipoprotein C1 | Flt3 | Hep3B2.1-7, HepG2, |
| | | C3A (HepG2/C3A), HuH-7, and |
| | | HuH-6 |
| Apolipoprotein C1 | phenylalanine | Hep3B2.1-7, HepG2, |
| | hyroxylase | C3A (HepG2/C3A), HuH-7, and |
| | | HuH-6 |
| Apolipoprotein H | Factor IX | Hep3B2.1-7, HepG2, |
| | | C3A (HepG2/C3A), HuH-7, and |
| | | HuH-6 |
| Apolipoprotein H | Factor VIII | Hep3B2.1-7, HepG2, |
| | | C3A (HepG2/C3A), HuH-7, and |
| | | HuH-6 |
| Apolipoprotein H | Erythropoietin | Hep3B2.1-7, HepG2, |
| | | C3A (HepG2/C3A), HuH-7, and |
| | | HuH-6 |
| Apolipoprotein H | Thrombopoietin | Hep3B2.1-7, HepG2, |
| | | C3A (HepG2/C3A), HuH-7, and |
| | | HuH-6 |
| Apolipoprotein H | Stem Cell Factor (KIT | Hep3B2.1-7, HepG2, |
| | ligand) | C3A (HepG2/C3A), HuH-7, and |
| | | HuH-6 |
| Apolipoprotein H | Interleukin 3 | Hep3B2.1-7, HepG2, |
| | | C3A (HepG2/C3A), HuH-7, and |
| | | HuH-6 |
| Apolipoprotein H | Interleukin 6 | Hep3B2.1-7, HepG2, |
| | | C3A (HepG2/C3A), HuH-7, and |
| | | HuH-6 |
| Apolipoprotein H | Granulocyte macrophage | Hep3B2.1-7, HepG2, |
| | colony stimulating factor | C3A (HepG2/C3A), HuH-7, and |
| | | HuH-6 |
| Apolipoprotein H | Flt3 | Hep3B2.1-7, HepG2, |
| | | C3A (HepG2/C3A), HuH-7, and |
| | | HuH-6 |
| Apolipoprotein H | phenylalanine | Hep3B2.1-7, HepG2, |
| | hyroxylase | C3A (HepG2/C3A), HuH-7, and |
| | | HuH-6 |
| Serpin peptidase | Factor IX | Hep3B2.1-7, HepG2, |
| inhibitor, clade A, member 1 | | C3A (HepG2/C3A), HuH-7, and |
| | | HuH-6 |
| Serpin peptidase | Factor VIII | Hep3B2.1-7, HepG2, |
| inhibitor, clade A, member 1 | | C3A (HepG2/C3A), HuH-7, and |
| | | HuH-6 |
| Serpin peptidase | Erythropoietin | Hep3B2.1-7, HepG2, |
| inhibitor, clade A, member 1 | | C3A (HepG2/C3A), HuH-7, and |
| | | HuH-6 |
| Serpin peptidase | Thrombopoietin | Hep3B2.1-7, HepG2, |
| inhibitor, clade A, member 1 | | C3A (HepG2/C3A), HuH-7, and |
| | | HuH-6 |
| Serpin peptidase | Stem Cell Factor (KIT | Hep3B2.1-7, HepG2, |
| inhibitor, clade A, member 1 | ligand) | C3A (HepG2/C3A), HuH-7, and |
| | | HuH-6 |
| Serpin peptidase | Interleukin 3 | Hep3B2.1-7, HepG2, |
| inhibitor, clade A, member 1 | | C3A (HepG2/C3A), HuH-7, and |
| | | HuH-6 |
| Serpin peptidase | Interleukin 6 | Hep3B2.1-7, HepG2, |
| inhibitor, clade A, member 1 | | C3A (HepG2/C3A), HuH-7, and |
| | | HuH-6 |
| Serpin peptidase | Granulocyte macrophage | Hep3B2.1-7, HepG2, |
| inhibitor, clade A, member 1 | colony stimulating factor | C3A (HepG2/C3A), HuH-7, and |
| | | HuH-6 |
| Serpin peptidase | Flt3 | Hep3B2.1-7, HepG2, |
| inhibitor, clade A, member 1 | | C3A (HepG2/C3A), HuH-7, and |
| | | HuH-6 |
| Serpin peptidase | phenylalanine | Hep3B2.1-7, HepG2, |
| inhibitor, clade A, member 1 | hyroxylase | C3A (HepG2/C3A), HuH-7, and |
| | | HuH-6 |
| Serpin peptidase | Factor IX | Hep3B2.1-7, HepG2, |
| inhibitor, clade A, member 3 | | C3A (HepG2/C3A), HuH-7, and |
| | | HuH-6 |
| Serpin peptidase | Factor VIII | Hep3B2.1-7, HepG2, |
| inhibitor, clade A, member 3 | | C3A (HepG2/C3A), HuH-7, and |
| | | HuH-6 |
| Serpin peptidase | Erythropoietin | Hep3B2.1-7, HepG2, |
| inhibitor, clade A, member 3 | | C3A (HepG2/C3A), HuH-7, and |
| | | HuH-6 |
| Serpin peptidase | Thrombopoietin | Hep3B2.1-7, HepG2, |
| inhibitor, clade A, member 3 | | C3A (HepG2/C3A), HuH-7, and |
| | | HuH-6 |
| Serpin peptidase | Stem Cell Factor (KIT | Hep3B2.1-7, HepG2, |
| inhibitor, clade A, member 3 | ligand) | C3A (HepG2/C3A), HuH-7, and |
| | | HuH-6 |
| Serpin peptidase | Interleukin 3 | Hep3B2.1-7, HepG2, |
| inhibitor, clade A, member 3 | | C3A (HepG2/C3A), HuH-7, and |
| | | HuH-6 |
| Serpin peptidase | Interleukin 6 | Hep3B2.1-7, HepG2, |
| inhibitor, clade A, member 3 | | C3A (HepG2/C3A), HuH-7, and |
| | | HuH-6 |
| Serpin peptidase | Granulocyte macrophage | Hep3B2.1-7, HepG2, |
| inhibitor, clade A, member 3 | colony stimulating factor | C3A (HepG2/C3A), HuH-7, and |
| | | HuH-6 |
| Serpin peptidase | Flt3 | Hep3B2.1-7, HepG2, |
| inhibitor, clade A, member 3 | | C3A (HepG2/C3A), HuH-7, and |
| | | HuH-6 |
| Serpin peptidase | phenylalanine | Hep3B2.1-7, HepG2, |
| inhibitor, clade A, member 3 | hyroxylase | C3A (HepG2/C3A), HuH-7, and |
| | | HuH-6 |
| Serpin peptidase | Factor IX | Hep3B2.1-7, HepG2, |
| inhibitor, clade A, member 7 | | C3A (HepG2/C3A), HuH-7, and |
| | | HuH-6 |
| Serpin peptidase | Factor VIII | Hep3B2.1-7, HepG2, |
| inhibitor, clade A, member 7 | | C3A (HepG2/C3A), HuH-7, and |
| | | HuH-6 |
| Serpin peptidase | Erythropoietin | Hep3B2.1-7, HepG2, |
| inhibitor, clade A, member 7 | | C3A (HepG2/C3A), HuH-7, and |
| | | HuH-6 |
| Serpin peptidase | Thrombopoietin | Hep3B2.1-7, HepG2, |
| inhibitor, clade A, member 7 | | C3A (HepG2/C3A), HuH-7, and |
| | | HuH-6 |
| Serpin peptidase | Stem Cell Factor (KIT | Hep3B2.1-7, HepG2, |
| inhibitor, clade A, member 7 | ligand) | C3A (HepG2/C3A), HuH-7, and |
| | | HuH-6 |
| Serpin peptidase | Interleukin 3 | Hep3B2.1-7, HepG2, |
| inhibitor, clade A, member 7 | | C3A (HepG2/C3A), HuH-7, and |
| | | HuH-6 |
| Serpin peptidase | Interleukin 6 | Hep3B2.1-7, HepG2, |
| inhibitor, clade A, member 7 | | C3A (HepG2/C3A), HuH-7, and |
| | | HuH-6 |
| Serpin peptidase | Granulocyte macrophage | Hep3B2.1-7, HepG2, |
| inhibitor, clade A, member 7 | colony stimulating factor | C3A (HepG2/C3A), HuH-7, and |
| | | HuH-6 |
| Serpin peptidase | Flt3 | Hep3B2.1-7, HepG2, |
| inhibitor, clade A, member 7 | | C3A (HepG2/C3A), HuH-7, and |
| | | HuH-6 |
| Serpin peptidase | phenylalanine | Hep3B2.1-7, HepG2, |
| inhibitor, clade A, member 7 | hyroxylase | C3A (HepG2/C3A), HuH-7, and |
| | | HuH-6 |
| Serpin peptidase | Factor IX | Hep3B2.1-7, HepG2, |
| inhibitor, clade C, member 1 | | C3A (HepG2/C3A), HuH-7, and |
| | | HuH-6 |
| Serpin peptidase | Factor VIII | Hep3B2.1-7, HepG2, |
| inhibitor, clade C, member 1 | | C3A (HepG2/C3A), HuH-7, and |
| | | HuH-6 |
| Serpin peptidase | Erythropoietin | Hep3B2.1-7, HepG2, |
| inhibitor, clade C, member 1 | | C3A (HepG2/C3A), HuH-7, and |
| | | HuH-6 |
| Serpin peptidase | Thrombopoietin | Hep3B2.1-7, HepG2, |
| inhibitor, clade C, member 1 | | C3A (HepG2/C3A), HuH-7, and |
| | | HuH-6 |
| Serpin peptidase | Stem Cell Factor (KIT | Hep3B2.1-7, HepG2, |
| inhibitor, clade C, member 1 | ligand) | C3A (HepG2/C3A), HuH-7, and |
| | | HuH-6 |
| Serpin peptidase | Interleukin 3 | Hep3B2.1-7, HepG2, |
| inhibitor, clade C, member 1 | | C3A (HepG2/C3A), HuH-7, and |
| | | HuH-6 |
| Serpin peptidase | Interleukin 6 | Hep3B2.1-7, HepG2, |
| inhibitor, clade C, member 1 | | C3A (HepG2/C3A), HuH-7, and |
| | | HuH-6 |
| Serpin peptidase | Granulocyte macrophage | Hep3B2.1-7, HepG2, |
| inhibitor, clade C, member 1 | colony stimulating factor | C3A (HepG2/C3A), HuH-7, and |
| | | HuH-6 |
| Serpin peptidase | Flt3 | Hep3B2.1-7, HepG2, |
| inhibitor, clade C, member 1 | | C3A (HepG2/C3A), HuH-7, and |
| | | HuH-6 |
| Serpin peptidase | phenylalanine | Hep3B2.1-7, HepG2, |
| inhibitor, clade C, member 1 | hyroxylase | C3A (HepG2/C3A), HuH-7, and |
| | | HuH-6 |

Disclosed in Table 6 are specific examples of proteins of interest, the target gene into which nucleic acid encoding the protein of interest can be inserted, and cells in which the expression system can be used.

TABLE 6
Specific Proteins of Interest, Non-Endogenous Genes
for Insertion, and Cells Useful in Combination

| Target Gene Name | Protein of Interest | Liver Cell Type |
|---|---|---|
| Albumin | Factor IX | C3A |
| Albumin | Factor VIII | C3A |
| Albumin | Erythropoietin | C3A |
| Albumin | Thrombopoietin | C3A |
| Albumin | Stem Cell Factor (KIT | C3A |
| | ligand) | |
| Albumin | Interleukin 3 | C3A |
| Albumin | Interleukin 6 | C3A |
| Albumin | Granulocyte macrophage | C3A |
| | colony stimulating factor | |
| Albumin | Flt3 | C3A |
| Albumin | phenylalanine hyroxylase | C3A |
| Alpha-1-microglobulin/ | Factor IX | C3A |
| bikunin precursor | | |
| Alpha-1-microglobulin/ | Factor VIII | C3A |
| bikunin precursor | | |
| Alpha-1-microglobulin/ | Erythropoietin | C3A |
| bikunin precursor | | |
| Alpha-1-microglobulin/ | Thrombopoietin | C3A |
| bikunin precursor | | |
| Alpha-1-microglobulin/ | Stem Cell Factor (KIT | C3A |
| bikunin precursor | ligand) | |
| Alpha-1-microglobulin/ | Interleukin 3 | C3A |
| bikunin precursor | | |
| Alpha-1-microglobulin/ | Interleukin 6 | C3A |
| bikunin precursor | | |
| Alpha-1-microglobulin/ | Granulocyte macrophage | C3A |
| bikunin precursor | colony stimulating factor | |
| Alpha-1-microglobulin/ | Flt3 | C3A |
| bikunin precursor | | |
| Alpha-1-microglobulin/ | phenylalanine hyroxylase | C3A |
| bikunin precursor | | |
| Alpha-2-HS-glycoprotein | Factor IX | C3A |
| Alpha-2-HS-glycoprotein | Factor VIII | C3A |
| Alpha-2-HS-glycoprotein | Erythropoietin | C3A |
| Alpha-2-HS-glycoprotein | Thrombopoietin | C3A |
| Alpha-2-HS-glycoprotein | Stem Cell Factor (KIT | C3A |
| | ligand) | |
| Alpha-2-HS-glycoprotein | Interleukin 3 | C3A |
| Alpha-2-HS-glycoprotein | Interleukin 6 | C3A |
| Alpha-2-HS-glycoprotein | Granulocyte macrophage | C3A |
| | colony stimulating factor | |
| Alpha-2-HS-glycoprotein | Flt3 | C3A |
| Alpha-2-HS-glycoprotein | phenylalanine hyroxylase | C3A |
| Apolipoprotein A2 | Factor IX | C3A |
| Apolipoprotein A2 | Factor VIII | C3A |
| Apolipoprotein A2 | Erythropoietin | C3A |
| Apolipoprotein A2 | Thrombopoietin | C3A |
| Apolipoprotein A2 | Stem Cell Factor (KIT | C3A |
| | ligand) | |
| Apolipoprotein A2 | Interleukin 3 | C3A |
| Apolipoprotein A2 | Interleukin 6 | C3A |
| Apolipoprotein A2 | Granulocyte macrophage | C3A |
| | colony stimulating factor | |
| Apolipoprotein A2 | Flt3 | C3A |
| Apolipoprotein A2 | phenylalanine hyroxylase | C3A |
| Apolipoprotein C1 | Factor IX | C3A |
| Apolipoprotein C1 | Factor VIII | C3A |
| Apolipoprotein C1 | Erythropoietin | C3A |
| Apolipoprotein C1 | Thrombopoietin | C3A |
| Apolipoprotein C1 | Stem Cell Factor (KIT | C3A |
| | ligand) | |
| Apolipoprotein C1 | Interleukin 3 | C3A |
| Apolipoprotein C1 | Interleukin 6 | C3A |
| Apolipoprotein C1 | Granulocyte macrophage | C3A |
| | colony stimulating factor | |
| Apolipoprotein C1 | Flt3 | C3A |
| Apolipoprotein C1 | phenylalanine hyroxylase | C3A |
| Apolipoprotein H | Factor IX | C3A |
| Apolipoprotein H | Factor VIII | C3A |
| Apolipoprotein H | Erythropoietin | C3A |
| Apolipoprotein H | Thrombopoietin | C3A |
| Apolipoprotein H | Stem Cell Factor (KIT | C3A |
| | ligand) | |
| Apolipoprotein H | Interleukin 3 | C3A |
| Apolipoprotein H | Interleukin 6 | C3A |
| Apolipoprotein H | Granulocyte macrophage | C3A |
| | colony stimulating factor | |
| Apolipoprotein H | Flt3 | C3A |
| Apolipoprotein H | phenylalanine hyroxylase | |
| Serpin peptidase inhibitor, | Factor IX | C3A |
| clade A, member 1 | | |
| Serpin peptidase inhibitor, | Factor VIII | C3A |
| clade A, member 1 | | |
| Serpin peptidase inhibitor, | Erythropoietin | C3A |
| clade A, member 1 | | |
| Serpin peptidase inhibitor, | Thrombopoietin | C3A |
| clade A, member 1 | | |
| Serpin peptidase inhibitor, | Stem Cell Factor (KIT | C3A |
| clade A, member 1 | ligand) | |
| Serpin peptidase inhibitor, | Interleukin 3 | C3A |
| clade A, member 1 | | |
| Serpin peptidase inhibitor, | Interleukin 6 | C3A |
| clade A, member 1 | | |
| Serpin peptidase inhibitor, | Granulocyte macrophage | C3A |
| clade A, member 1 | colony stimulating factor | |
| Serpin peptidase inhibitor, | Flt3 | C3A |
| clade A, member 1 | | |
| Serpin peptidase inhibitor, | phenylalanine hyroxylase | C3A |
| clade A, member 1 | | |
| Serpin peptidase inhibitor, | Factor IX | C3A |
| clade A, member 3 | | |
| Serpin peptidase inhibitor, | Factor VIII | C3A |
| clade A, member 3 | | |
| Serpin peptidase inhibitor, | Erythropoietin | C3A |
| clade A, member 3 | | |
| Serpin peptidase inhibitor, | Thrombopoietin | C3A |
| clade A, member 3 | | |
| Serpin peptidase inhibitor, | Stem Cell Factor (KIT | C3A |
| clade A, member 3 | ligand) | |
| Serpin peptidase inhibitor, | Interleukin 3 | C3A |
| clade A, member 3 | | |
| Serpin peptidase inhibitor, | Interleukin 6 | C3A |
| clade A, member 3 | | |
| Serpin peptidase inhibitor, | Granulocyte macrophage | C3A |
| clade A, member 3 | colony stimulating factor | |
| Serpin peptidase inhibitor, | Flt3 | C3A |
| clade A, member 3 | | |
| Serpin peptidase inhibitor, | phenylalanine hyroxylase | C3A |
| clade A, member 3 | | |
| Serpin peptidase inhibitor, | Factor IX | C3A |
| clade A, member 7 | | |
| Serpin peptidase inhibitor, | Factor VIII | C3A |
| clade A, member 7 | | |
| Serpin peptidase inhibitor, | Erythropoietin | C3A |
| clade A, member 7 | | |
| Serpin peptidase inhibitor, | Thrombopoietin | C3A |
| clade A, member 7 | | |
| Serpin peptidase inhibitor, | Stem Cell Factor (KIT | C3A |
| clade A, member 7 | ligand) | |
| Serpin peptidase inhibitor, | Interleukin 3 | C3A |
| clade A, member 7 | | |
| Serpin peptidase inhibitor, | Interleukin 6 | C3A |
| clade A, member 7 | | |
| Serpin peptidase inhibitor, | Granulocyte macrophage | C3A |
| clade A, member 7 | colony stimulating factor | |
| Serpin peptidase inhibitor, | Flt3 | C3A |
| clade A, member 7 | | |
| Serpin peptidase inhibitor, | phenylalanine hyroxylase | C3A |
| clade A, member 7 | | |
| Serpin peptidase inhibitor, | Factor IX | C3A |
| clade C, member 1 | | |
| Serpin peptidase inhibitor, | Factor VIII | C3A |
| clade C, member 1 | | |
| Serpin peptidase inhibitor, | Erythropoietin | C3A |
| clade C, member 1 | | |
| Serpin peptidase inhibitor, | Thrombopoietin | C3A |
| clade C, member 1 | | |
| Serpin peptidase inhibitor, | Stem Cell Factor (KIT | C3A |
| clade C, member 1 | ligand) | |
| Serpin peptidase inhibitor, | Interleukin 3 | C3A |
| clade C, member 1 | | |
| Serpin peptidase inhibitor, | Interleukin 6 | C3A |
| clade C, member 1 | | |
| Serpin peptidase inhibitor, | Granulocyte macrophage | C3A |
| clade C, member 1 | colony stimulating factor | |
| Serpin peptidase inhibitor, | Flt3 | C3A |
| clade C, member 1 | | |
| Serpin peptidase inhibitor, | phenylalanine hyroxylase | C3A |
| clade C, member 1 | | |

The non-endogenous gene into which the nucleic acid is inserted can be albumin, or other genes provide herein. When the gene is not albumin, it can be alpha-1-microglobulin/bikunin precursor; alpha-2-HS-glycoprotein; alphafetoprotein; apolipoprotein A2; apolipoprotein C1; apolipoprotein H; fibrinogen gamma chain; serpin peptidase inhibitor, clade A, member 1; serpin peptidase inhibitor, clade A, member 3; serpin peptidase inhibitor, clade A, member 7; serpin peptidase inhibitor, clade C, member 1; or transferrin.

For example, a nucleic acid encoding Factor IX can be inserted into the albumin gene, and a nucleic acid encoding Factor VIII can inserted into the alpha 1 antitrypsin gene, so that the cell produces two different proteins of interest. In another example, a nucleic acid encoding erythropoietin can be inserted (or the endogenous coding gene can be replaced) into/by the albumin gene, and a nucleic acid encoding stem cell factor can be inserted into the alpha 1 antitrypsin gene, and a nucleic acid encoding interleukin 3 can be inserted into alpha 1 microglobulin gene, so that three different proteins of interest can be produced from three different genes. Therefore, a single engineered cell can produce three different proteins of interest. In another example, the gene for Factor VIII can be inserted into the albumin locus and the gene for von Willebrand Factor (VWF) can be inserted into the alpha-1-antitrypsin gene. VWF stabilizes Factor VIII and prevents its degradation in the culture fluid. Another example can be heterodimeric proteins, such as hemoglobin. The most common form of adult hemoglobin contains two alpha chains and two beta chains. These two subunits, alpha and beta, are coded by different genes on different chromosomes. By inserting an alpha chain in the albumin locus and a beta chain in the alpha-1-antitrypsin gene, a functional hemoglobin can be generated.

The engineered cells disclosed herein can produce the protein of interest at 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 or more picograms/cell/day.

The cell can be optimized for production in a variety of ways. For example, the coding sequence of cDNA encoding the non-albumin protein can be optimized (Fath, S., Bauer, A. P., Lisa, M., Spriestersbach, A., Maertens, B., Hahn, P., et al. (2011). Multiparameter RNA and Codon Optimization: A Standardized Tool to Assess and Enhance Autologous Mammalian Gene Expression. PLoS ONE, 6(3), e17596). Each of the nucleotide triplets in an RNA directs a particular charged transfer RNA to add its cognate amino acid to the growing peptide chain. Many of the amino acids have two or more transfer RNAs. By using the triplet that codes for the most abundant transfer RNA, protein synthesis can be increased. In another example, an intervening sequence can be included in the nucleic acid encoding the non-albumin protein as described herein and in NOTT, A. (2003). A quantitative analysis of intron effects on mammalian gene expression. RNA, 9(5), 607-617. And in another example, the cell can be optimized for production of the non-albumin protein by inclusion of a stabilizing 3′ untranslated region within the nucleic acid encoding the non-albumin protein. These optimization methods can produce significantly higher amounts of the protein of interest when compared to a control. For example, when the control comprises a system utilizing the same protein of interest (non-albumin gene) and the same insertion site, production of the protein of interest can be 2, 3, 4, 5, 6, 7, 8, 9, 10, 20, 30, 40, 50, 60, 70, 80, 90, 100, or more times higher than if one or more of the optimization methods were not used.

Also disclosed is a protein produced by a nucleic acid encoding a non-albumin protein operably inserted into an albumin gene locus in a hepatocyte or hepatocyte-derived cell line where the protein is glycosylated, such as Factor IX. The human liver cell system described here produces a human glycosylation pattern.

The protein produced from the engineered cells disclosed herein can be combined with a pharmaceutically acceptable carrier. Examples of pharmaceutically acceptable carriers can allow for intravenous administration, intraperitoneal administration, intramuscular administration, intracoronary administration, intraarterial administration, intradermal administration, subcutaneous administration, transdermal delivery, intratracheal administration, subcutaneous administration, intraarticular administration, intraventricular administration, inhalation, intracerebral, nasal, naval, oral, intraocular, pulmonary administration, impregnation of a catheter, by suppository and direct injection into a tissue, or systemically absorbed topical or mucosal administration. A person of skill in the art can envision that the protein produced can be combined with any pharmaceutically acceptable carrier known in the art.

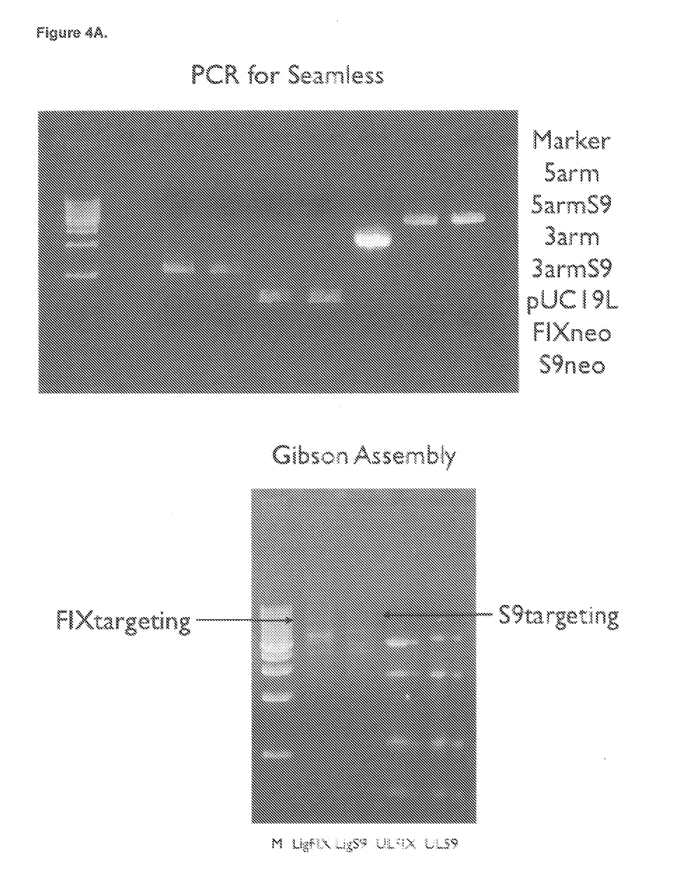

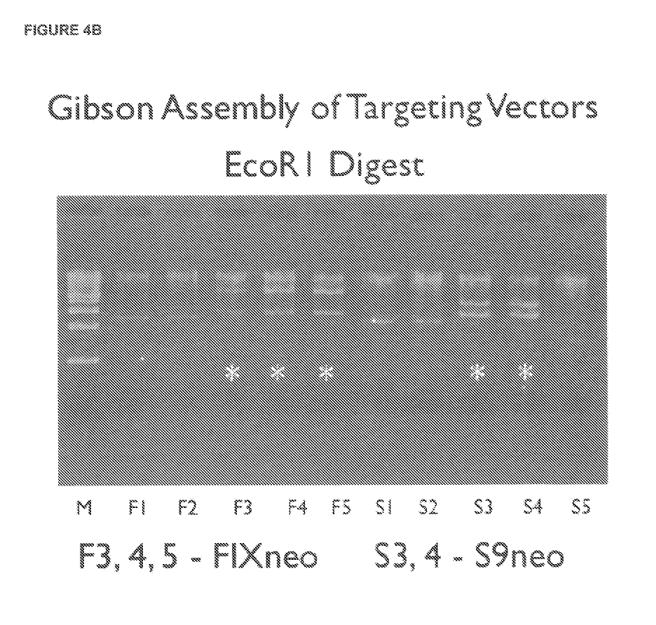

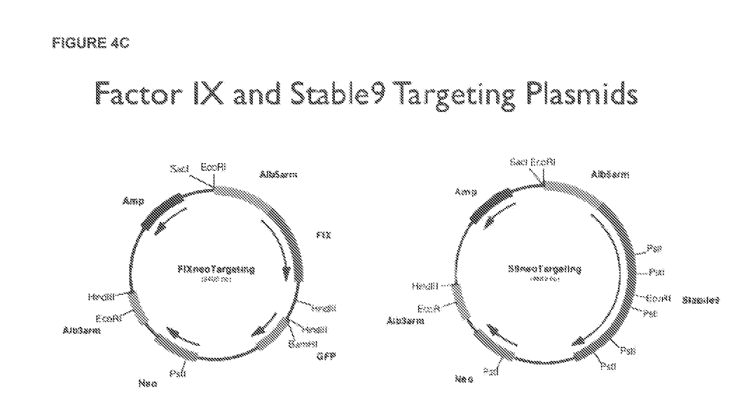

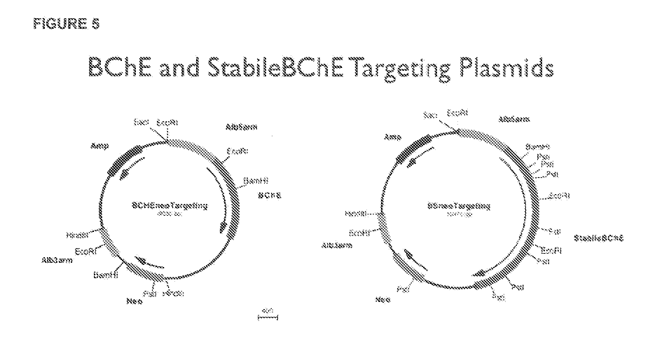

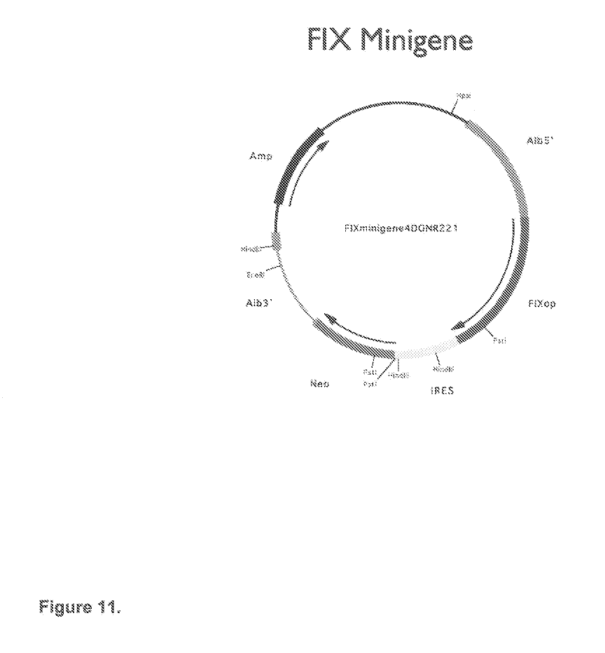

Also disclosed herein is a system which is useful for producing a protein from the engineered cells disclosed herein. This system can be used in fully disposable bioreactors housed in mobile clean rooms. When the culture period is finished, the disposable reactor can be discarded and replaced quickly, making it possible to produce more protein in less time. This system can also be used in a mobile clean room. These are prefabricated, class 100 clean rooms that can be inserted into general building space as opposed to purpose built clean rooms that are part of the building structure. The combined use of disposable bioreactors in mobile clean rooms can dramatically lower the cost of producing proteins according to the US Food and Drug Administration current Good Manufacturing Practices. For example, provided herein is a small, multiproduct facility, capable of producing proteins on the scale required for Factor IX, consisting of four mobile clean rooms (MCRs), installed in standard class 100,000 warehouse space. One MCR would be used for cell expansion, a second for production, the third for purification and the fourth for fill/finish and vialing. Using a disposable type bioreactor and other disposables in this process, a facility suitable for cGMP can be dramatically less expensive than the traditional stainless steel, clean-in-place type facility usually employed. Moreover, the use of disposables allows rapid change over from one production run to the next or even from one product to the next. Finally, construction and validation of such a facility requires less than 18 months as opposed to the three to five years for traditional facilities. Further disclosed herein is a method of producing a non-albumin protein, the method comprising a) culturing the engineered cells disclosed herein; and b) allowing the cell to produce the non-albumin protein. The albumin gene, or other non-endogenous gene can be excised, either partially or completely, prior to producing and incorporating the non-albumin gene. For example, nucleic acid constructs can be created that comprise a functional gene, with a stop codon, followed by sequences homologous to the insertion point, such as shown for FIXneo, in FIG. 4A. This construct inserts into the first coding sequence of the human albumin gene and interrupts the gene but leaves the remaining 12 kilobases of the albumin gene intact. Another form of construct can be created where sequences homologous to the 5′ end of the non-endogenous gene and sequences homologous to the 3′ end of the non-endogenous gene are used, such as shown in the FIXminigene construct shown in FIG. 11. Since the construct inserts via homologous recombination, the sequences internal to the construct are deleted. In the case of the FIXminigene, only a piece of the first coding sequence and the final two coding sequences plus the final intervening sequence of the albumin gene are retained. In another example, the albumin or other non-endogenous gene can be expressed as a fusion protein with the protein of interest (non-albumin gene). The protein of interest (non-albumin gene) can also be purified. In the instance where only the protein of interest, not a fusion protein, is produced, the protein is purified by standard biochemical techniques such as column chromatography. In the case of a fusion protein, the protein of interest can be purified by techniques used to purify the fusion partner. For example, albumin is retained on columns of Cibachron blue F3GA. A fusion protein containing factor IX fused to albumin or SFP can be purified by retention on Cibachron blue F3GA.

### Synthetic Fusion Proteins

### General

The present invention is based, in part, on the discovery that a protein of interest (e.g., a polypeptide, antibody, or peptide, or fragments and variants thereof) may be stabilized to extend the shelf-life and/or retain the protein of interest's activity for extended periods of time in solution (or in a pharmaceutical composition) in vitro and/or in vivo, by genetically fusing or chemically conjugating the protein of interest, polypeptide or peptide to two or more domains of proteins selected from the human albumin gene superfamily (referred to herein as the synthetic fusion protein, or SFP). This fusion protein with a protein of interest together makes up what is referred to herein as an SFP-POI, which sufficient to stabilize the protein and its activity.

The SFP serves two purposes. It is designed to maximize protein half life in vivo through binding to the neonatal Fc receptor (FcRN). It is also designed to allow facile, antibody free purification of the fused product without regard to the function of the therapeutic protein.

The other parts of this comprehensive production system utilize the human albumin locus in a human liver derived cell line. In that light, a second consideration in the design of SFP was the ability to distinguish the protein from human albumin but minimize the possibility of immune response to a synthetic protein. Simply altering the amino acids of albumin to maximize binding has been shown to be effective but the altered protein is essentially indistinguishable from albumin. Fusion of a desired protein to an altered albumin would necessitate purification procedures to be designed around the desired protein since the liver derived cells produce large amount of albumin. A wholly synthetic protein that binds to FcRN could be designed but would almost certainly provoke an unwanted immune response. SFP combines pieces of several of the members of the human albumin gene superfamily to accomplish the combined goal of long half-life, ease of purification and minimal immune response.

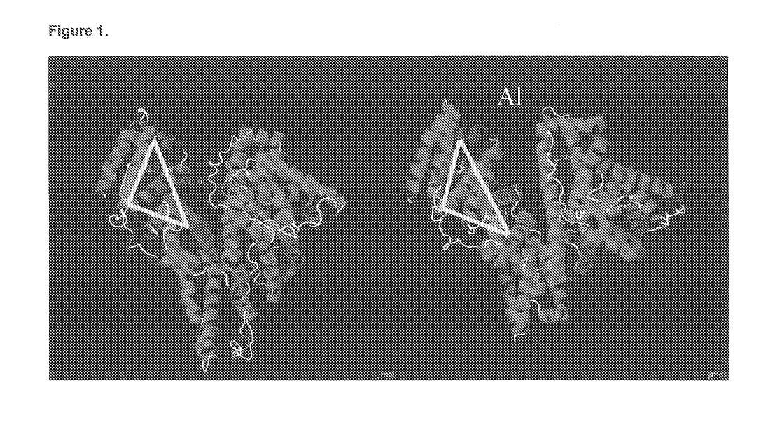

There are four genes in the albumin superfamily. These four genes, consisting of albumin, alphafetoprotein, vitamin D binding protein (also known as Gc globulin) and afamin, share similar structure and code for similarly shaped proteins (Peters, T. (1996) ALL ABOUT ALBUMIN, Academic Press, San Diego, Calif., 423). FIG. 1 shows a computer model of SFP2 compared to the known structure of human albumin. While the proteins are structurally similar, SFP2 shares only 53% homology to albumin on an amino acid basis. Specifically, the section of SFP2 that corresponds to Domain 1 of the human albumin, is derived from the Vitamin D binding protein and contains the vitamin D binding pocket. In this way, a vitamin D affinity column can be used to purify the fusion protein away from albumin, followed by ion exchange chromatography to separate it from native Vitamin D binding protein. The fusion protein can be purified with only minimal consideration of the properties of the desired partner.

Two versions of SFP are described here: SFP2, based on vitamin D binding protein and alphafetoprotein and SFP3, based on afamin and alphafetoprotein. Proteins using SFP2 can be purified on a vitamin D affinity column whereas proteins using SFP3 can be purified on a vitamin E affinity column.

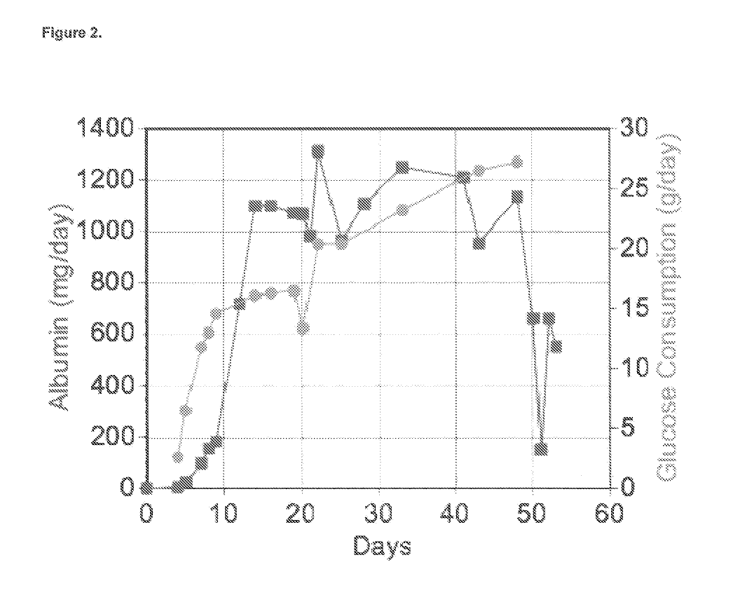

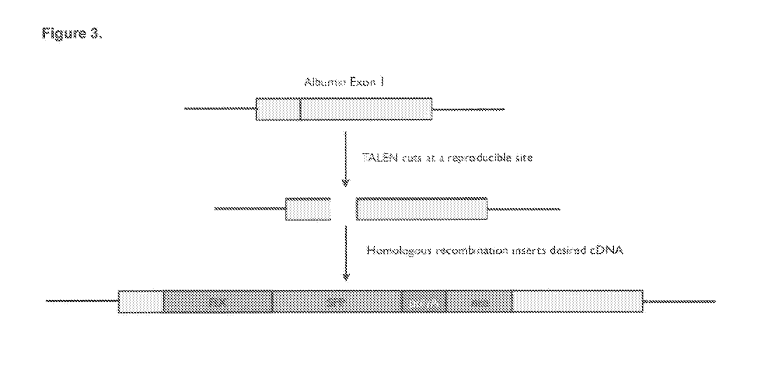

The human liver is capable of massive protein synthesis and produces 30 to 50 grams of protein per day (Peters, T. (1996) ALL ABOUT ALBUMIN, Academic Press, San Diego, Calif., 423). Albumin is the major protein produced by the liver, comprising about 15% of the total output (Peavy, D E, et al. (1978) Correlation of albumin production rates and albumin mRNA levels in livers of normal, diabetic, and insulin-treated diabetic rats. Proc. Natl. Acad. Sci. 75, 5879-5883). Other highly synthesized serum proteins include alpha-1-antitrypsin and transferrin (Bowman, B H (1993) HEPATIC PLASMA PROTEINS, Academic Press, San Diego, Calif.). Most human liver cell lines recapitulate this synthesis, although not usually at the level of primary hepatocytes derived directly from a liver. FIG. 2 shows human albumin synthesis from one such cell line, HepG2/C3A (C3A) (Kelly, J H (1994) U.S. Pat. No. 5,290,684). This chart shows that about 100 g of C3A produces 1 gram of human albumin per day. Moreover, the cells were capable of this production for a sustained period, over a month. The albumin gene is highly transcribed in the liver and liver derived cell lines and produces a very stable, highly translated mRNA. Additionally, the hepatocyte is capable of processing and secreting this large mass of protein (Peters, T. (1996) ALL ABOUT ALBUMIN, Academic Press, San Diego, Calif., 423). Using homologous recombination, any gene can be inserted into this locus thereby switching the cell from production of albumin to production of the desired protein at a similar level. FIG. 3 shows a diagram of this process.

Transcription activator like elements fused to restriction endonucleases (TALENS) allow very specific insertion into essentially any known sequence (Miller, J C, et al. (2010) A TALE nuclease architecture for efficient genome engineering. Nat. Biotechnol. 29, 143-148). Two complementary TALENS are created, one binding on either side of the desired insertion site. Upon binding, the nuclease dimerizes and makes a double stranded cut at the specific site. When the TALENS are used in combination with a targeting vector containing sequences homologous to the insertion site but carrying the desired sequence, they insert cleanly and specifically into the chosen site. In this way, any cDNA or gene could be inserted into the human albumin gene such that transcription and secretion are not disturbed.

Albumin is used here as a primary example but other genes could be easily used by designing specific TALENS. Moreover, since the TALENS are site specific, double and triple insertions are possible using separate target genes, such as albumin and alpha-1-antitrypsin. This may be desirable when two protein are needed to form a complex, such as in the case of heteromeric proteins consisting of two different subunits.

Site specific insertion could also be accomplished using zinc finger nucleases (Durai, S, et al. (2005). Zinc finger nucleases:custom-designed molecular scissors for genome engineering of plant and mammalian cells. Nuc. Acids. Res. 33, 5978-5990).

The present invention relates generally to synthetic fusion proteins and methods of treating, preventing, or ameliorating diseases or disorders. As used herein, “synthetic fusion protein (SFP)” refers to a peptide comprising multiple domains, where at least two domains (or fragments or variants thereof) are selected from different members of the human albumin superfamily. The SFP can then be fused to a protein of interest, which is referred to as an SFP-POI. For example, the domains that make up the albumin superfamily portion of the SFP can be selected from any of the members of the albumin superfamily, including but not limited to albumin, alpha-fetoprotein, vitamin D-binding protein and afamin.

The SFP can comprise two, three, or more domains. For example, one or two domains can be from the vitamin D-binding protein. When this is the case, the polypeptide can be capable of binding vitamin D. In another example, at least one domain can be derived from alphafetoprotein. In another example, at least one domain can be derived from afamin. For example, two domains can be derived from afamin. The peptide can, for example, bind vitamin E.

Some examples of SFP domains can include the following combinations:

DBP1-DBP2-ALB3

DBP1-DBP2-AFP3

DBP1-DBP2-AFM3

AFM1-AFM2-ALB2

AFM1-AFM2-AFP3

### Wherein DBP is Vitamin D Binding Protein, AFP is Alphafetoprotein, ALB is Albumin, and AFM is Afamin.

The invention comprises at least a fragment or variant of a protein of interest and an albumin superfamily portion, which are associated with one another, preferably by genetic fusion (i.e., the fusion protein is generated by translation of a nucleic acid in which a polynucleotide encoding all or a portion of a protein of interest is joined in-frame with a polynucleotide encoding the albumin superfamily portion) or chemical conjugation to one another. The protein of interest, when fused to the albumin superfamily protein or SFP portion, may be referred to as a the “fusion protein.”

In one embodiment, the invention provides a SFP-POI comprising, or alternatively consisting of, a protein of interest and a synthetic fusion protein. In other embodiments, the invention provides an SFP-POI comprising, or alternatively consisting of, a biologically active and/or therapeutically active fragment of a therapeutic protein and an albumin superfamily protein portion. In other embodiments, the invention provides an SFP-POI comprising, or alternatively consisting of, a biologically active and/or therapeutically active variant of a protein of interest and an albumin superfamily protein portion. In preferred embodiments, the albumin superfamily protein portion component of the SFP-POI is the mature portion of any one or more members of the human albumin superfamily, including but not limited to albumin, alpha-fetoprotein, vitamin D-binding protein and afamin.

In further embodiments, the invention provides SFP-POI comprising, or alternatively consisting of, a protein of interest, and a biologically active and/or therapeutically active fragment of a domain of one or more members of the albumin superfamily. In a further preferred embodiment, the protein of interest portion of the SFP-POI is the extracellular soluble domain of the protein of interest. In an alternative embodiment, the protein of interest is the active form of the protein.

In further embodiments, the invention provides an SFP-POI comprising, or alternatively consisting of, a biologically active and/or therapeutically active fragment or variant of a protein of interest and a biologically active and/or therapeutically active fragment or variant of one of more domains of a protein selected from the albumin superfamily. In preferred embodiments, the invention provides an SFP-POI comprising, or alternatively consisting of, the mature portion of a protein of interest and a functionally mature portion of one or more fusion proteins selected from the human albumin superfamily.

Disclosed herein are compositions and methods for delivery of a protein of interest (e.g., a polypeptide, antibody, or peptide, or fragments and variants thereof), where the protein of interest is stabilized to extend the shelf-life and/or retain the protein of interest's activity for extended periods of time in solution (or in a pharmaceutical composition) in vitro and/or in vivo, by genetically fusing or chemically conjugating the protein of interest, polypeptide or peptide to all or a portion of the synthetic fusion protein to stabilize the protein of interest and its activity.

### Proteins of Interest

As stated above, an SFP-POI comprises at least a fragment or variant of a protein of interest and at least a fragment or variant of one or more domains of an albumin superfamily protein, which are associated with one another, preferably by genetic fusion or chemical conjugation.

As used herein, “protein of interest” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics. (The terms peptides, proteins, and polypeptides are used interchangeably herein.) It is specifically contemplated that the term “protein of interest” encompasses antibodies and fragments and variants thereof. Thus an SFP-POI of the invention may contain at least a fragment or variant of a protein of interest, and/or at least a fragment or variant of an antibody. Additionally, the term “protein of interest” may refer to the endogenous or naturally occurring correlate of a protein of interest.

By a polypeptide displaying a “therapeutic activity” or a protein that is “therapeutically active” is meant a polypeptide that possesses one or more known biological and/or therapeutic activities associated with a protein of interest such as one or more of the proteins of interest described herein or otherwise known in the art. As a non-limiting example, a “protein of interest” is a protein that is useful to treat, prevent or ameliorate a disease, condition or disorder. As a non-limiting example, a “protein of interest” may be one that binds specifically to a particular cell type (normal (e.g., lymphocytes) or abnormal e.g., (cancer cells)) and therefore may be used to target a compound (drug, or cytotoxic agent) to that cell type specifically.

In another non-limiting example, a “protein of interest” is a protein that has a biological activity, and in particular, a biological activity that is useful for treating, preventing or ameliorating a disease. A non-inclusive list of biological activities that may be possessed by a protein of interest includes, enhancing the immune response, promoting angiogenesis, inhibiting angiogenesis, regulating hematopoietic functions, stimulating nerve growth, enhancing an immune response, inhibiting an immune response, or any one or more of the biological activities described herein.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

Examples of useful assays for particular proteins of interest include, but are not limited to, Human chorionic gonadotropin (hCG receptor binding and activation assay: J Biol Chem 268(28):20851-4 (1993)), Leptin (cell-based assay: Protein Expr Purif 4(3):335-42 (1998)), B-glucocerebrosidase (fluorometric assay: Daniels et al., Clin Chim Acta. 106(2):155-63 (1980) and Johnson et al., Clin Chim Acta. 102(1):91-7 (1980)), DNASE (DNA degradation assay: J Biochem (Tokyo) 92(4):1297-303 (1982)), Follicle Stimulating Hormone (cAMP assay: J Reprod Immunol 49(1):1-19 (2001)), TNF Receptor (PIP5K assay: J Biol Chem 272(9):5861-5870 (1997)), Urokinase (plasminogen cleavage assay: (Sazonova et al., J Biol Chem 2001 Jan. 18 (electronic prepublication)), Decorin (collagen fibril stability assay: Cell Mol Life Sci 57(5):859-863 (2000) or an in vitro cell adhesion assay: J Cell Biochem 67(1):75-83 (1997)), Osteoprotegrin (co-culture assay for osteoclastogenesis, bone resorption assay dentine resorption assay, or fibroblast proliferation assay: FASEB J. 12:845-854 (1998)), Human luteinizing hormone (in vitro fluorescence assay: Endocrinology 141(6):2220-2228 (2000)), Tie-2 (phosphorylation assay: Int Immunol 10(8):1217-1227 (1998)), t-PA (Wallen, R, Biochemistry of plasminogen. In: Kline D. L., Reddy, K. N. N., eds. Fibrinolysis. Boca Raton, Fla.: CRC Press, 1980:1-25; Saksela, and Rifkin, Annu Rev Cell Biol 4:93-126 (1988); Womack et al., Med Sci Sports Exerc 33(2):214-9 (2001).

Proteins of such as cell surface and secretory proteins, are often modified by the attachment of one or more oligosaccharide groups. The modification, referred to as glycosylation, can dramatically affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. N-acetylneuramic acid (also known as sialic acid) is usually the terminal residue of both N-linked and O-linked oligosaccharides. Variables such as protein structure and cell type influence the number and nature of the carbohydrate units within the chains at different glycosylation sites. Glycosylation isomers are also common at the same site within a given cell type.

For example, several types of human interferon are glycosylated. Natural human interferon alpha2 is O-glycosylated at threonine 106, and N-glycosylation occurs at asparagine 72 in interferon alpha14 (Adolf et al., J. Biochem 276:511 (1991); Nyman T A et al., J. Biochem 329:295 (1998)). The oligosaccharides at asparagine 80 in natural interferon-beta/alpha may play an important factor in the solubility and stability of the protein, but may not be essential for its biological activity. This penults the production of an unglycosylated analog (interferon-(beta 1b) engineered with sequence modifications to enhance stability (Hosoi et al., J. Interferon Res. 8:375 (1988; Karpusas et al., Cell Mol Life Sci 54:1203 (1998); Knight, J. Interferon Res. 2:421 (1982); Runkel et al., Pharm Res 15:641 (1998); Lin, Dev. Biol. Stand. 96:97 (1998))1. Interferon-.gamma. contains two N-linked oligosaccharide chains at positions 25 and 97, both important for the efficient formation of the bioactive recombinant protein, and having an influence on the pharmacokinetic properties of the protein (Sareneva et al., Eur. J. Biochem 242:191 (1996); Sareneva et al., Biochem J. 303:831 (1994); Sareneva et al., J. Interferon Res. 13:267 (1993)). Mixed O-linked and N-linked glycosylation also occurs, for example in human erythropoietin, N-linked glycosylation occurs at asparagine residues located at positions 24, 38 and 83 while O-linked glycosylation occurs at a serine residue located at position 126 (Lai et al., J. Biol. Chem. 261:3116 (1986); Broudy et al., Arch. Biochem. Biophys. 265:329 (1988)).

Proteins of interest, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g. in E. coli or glycosylation-deficient yeast. These approaches are described in more detail below and are known in the art.

Proteins of interest include, but are not limited to, TNF Receptor, enzymes (such as, for example, urokinase, B-glucocerebrosidase), growth factors (such as, for example, epidermal growth factor, FGF-1, fibroblast growth factor-2, nerve growth factor, platelet-derived growth factor, VEGF-1), interleukins (such as, for example, IL-1, IL-4, IL-8, IL-10, IL-11, IL-12), interleukin receptors (such as, for example, interleukin-4 receptor); interferons (e.g., interferon gamma, interferon omega); transforming growth factors (including, but not limited to, TGF-beta, TGF-beta-1, TGF-beta-3); tumor necrosis factors (such as, for example, TNF alpha), and hormones (such as, for example, gonadotropin, Human luteinizing hormone, Follicle Stimulating Hormone). These proteins and nucleic acid sequences encoding these proteins are well known and available in public databases such as Chemical Abstracts Services Databases (e.g., the CAS Registry), GenBank, and GenSeq.

Other proteins of interest include coagulation factor IX, butyrylcholinesterase, coagulation factor VIII, coagulation factor Viia, alpha-1-antitrypsin, antithrombin III, phenylalanine hydroxylase, erythropoietin, growth hormone, granulocyte colony stimulating factor, interferon beta, and atrial natriuretic peptide.

The protein of interest need not be a therapeutic, and in fact can be used as a vaccine antigen. The protein of interest can also be a single chain variable fragment.

### Polypeptide and Polynucleotide Fragments and Variants

### Fragments

The present invention is further directed to fragments of the proteins of interest described herein as well as fragments of individual domains selected from members of the human albumin superfamily, as well as functional fragments of the entire SFP-POI molecule.

Even if deletion of one or more amino acids from the N-terminus of a protein results in modification or loss of one or more biological functions of the protein of interest, or individual domains selected from members of the human albumin superfamily (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained when less than the majority of the residues of the complete polypeptide are removed from the N-terminus. Whether a particular polypeptide lacking N-terminal residues of a complete polypeptide retains such immunologic activities can readily be determined by routine methods described herein and otherwise known in the art. It is not unlikely that a mutein with a large number of deleted N-terminal amino acid residues may retain some biological or immunogenic activities. In fact, peptides composed of as few as six amino acid residues may often evoke an immune response.

Accordingly, fragments of a molecule or protein of interest, include the full length protein as well as polypeptides having one or more residues deleted from the amino terminus of the amino acid sequence of the reference polypeptide, are contemplated herein. In addition, fragments of proteins from the human albumin superfamily polypeptides corresponding to an albumin protein portion of an SFP of the invention, including the full length protein, or domains thereof, as well as polypeptides having one or more residues deleted from the amino terminus of the amino acid sequence of the reference polypeptide (i.e., albumin superfamily protein), are herein contemplated.

Moreover, fragments of SFPs of the invention, include the full length SFP as well as polypeptides having one or more residues deleted from the amino terminus of the SFP. Also as mentioned above, even if deletion of one or more amino acids from the N-terminus or C-terminus of a reference polypeptide results in modification or loss of one or more biological functions of the protein, other functional activities (e.g., biological activities, ability to multimerize, ability to bind a ligand) and/or therapeutic activities may still be retained. For example, the ability of polypeptides with C-terminal deletions to induce and/or bind to antibodies which recognize the complete or mature forms of the polypeptide generally will be retained when less than the majority of the residues of the complete or mature polypeptide are removed from the C-terminus. Whether a particular polypeptide lacking the N-terminal and/or C-terminal residues of a reference polypeptide retains therapeutic activity can readily be determined by routine methods described herein and/or otherwise known in the art.

The present invention further provides polypeptides having one or more residues deleted from the carboxy terminus of the amino acid sequence of a protein of interest. In addition, the present invention provides polypeptides having one or more residues deleted from the carboxy terminus of the amino acid sequence of albumin superfamily protein portion. Polynucleotides encoding these polypeptides are also encompassed by the invention.

The present application is also directed to proteins containing polypeptides at least 50%, 55%, 60%, 65%, 70%, 75%, 80%, 85%, 90%, 95%, 96%, 97%, 98% or 99% identical to a reference polypeptide sequence (e.g., a protein of interest or the albumin superfamily fusion protein portion) set forth herein, or fragments thereof. In preferred embodiments, the application is directed to proteins comprising polypeptides at least 50%, 55%, 60%, 65%, 70%, 75%, 80%, 85%, 90%, 95%, 96%, 97%, 98% or 99% identical to reference polypeptides having the amino acid sequence of N- and C-terminal deletions as described above. Polynucleotides encoding these polypeptides are also encompassed by the invention.

For example, the albumin superfamily protein can be derived from domains of different albumin superfamily members (albumin, alphafetoprotein, afamin, or vitamin D binding protein). Each domain can have 5, 10, 15, 20, 25, 30, 35, 40, 45, 50, 55, 60, 65, 70, 75, 80, 85, 90, 95, 96, 97, 98, 99, or 100%, or any amount in between, sequence similarity to the domain for the native albumin superfamily member. For example, if the albumin superfamily protein comprises two domains from afamin and one domain from vitamin D binding protein, the first domain from afamin can have 90% identity to the native afamin domain sequence, the second domain from afamin can have 84% homology with the native afamin domain sequence, and the third domain can have 100% sequence homology with the native vitamin D binding protein domain.

Relatedly, the polypeptide comprising one or more domains or fragments thereof from the human albumin superfamily can have less than 80%, 75%, 70%, 65%, 60%, 55%, 50%, 45%, 40%, 35%, 30%, 25%, 20%, 15%, 10%, or 5% or less homology to the human albumin protein. As described above, the human albumin superfamily portion can be a compilation of domains from multiple members of the albumin superfamily. The albumin superfamily protein can comprise one domain from albumin, and one, two, or three domains from other superfamily members. Relatedly, the polypeptide comprising one or more domains or fragments thereof from the human albumin superfamily can have less than 80%, 75%, 70%, 65%, 60%, 55%, 50%, 45%, 40%, 35%, 30%, 25%, 20%, 15%, 10%, or 5% or less homology to the human alphafetoprotein. As described above, the human albumin superfamily portion can be a compilation of domains from multiple members of the albumin superfamily. The albumin superfamily protein can comprise one domain from albumin, and one, two, or three domains from other superfamily members.

By way of example, shown below is the third domain of the synthetic fusion protein (SFP2) aligned with the same region in AFP. Each of the amino acid changes were done intentionally to maximize binding to the neonatal Fc receptor. The three histidines marked in red are essential for FCRN binding. The first, at AA 479 in SFP2, is conserved in AFP but the surrounding sequence was modified to match human albumin. The second, at AA 525 in SFP2, is also conserved but the surrounding sequence was modified to match the mouse albumin sequence. This gives SFP2 binding similar to mouse. The third, at AA 550 in SFP2, is a glutamine in AFP. It was changed to the histidine that is present in human albumin at this position. (SEQ ID NO: 5 is SFP2; SEQ ID NO: 6 is the AFP).

Third domain of SFP2 compared to AFP sequence

Query 407 ---CADYSENTFYYLQNAFLVAYTKKAPQLTSSELMAITRKMAATAATCCQLSEDKLLAC 463
C + + YYLQNAFLVAYTKKAPQLTSSELMAITRKMAATAATCCQLSEDKLLAC
Sbjct 413 KRSCGLFQKLGEYYLQNAFLVAYTKKAPQLTSSELMAITRKMAATAATCCQLSEDKLLAC 472

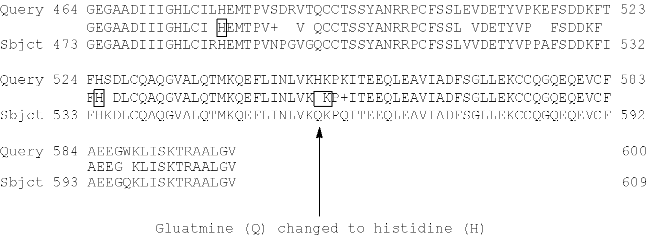

Another example is found below. The sequence of SFP2 aligned with DBP over the first two domains (DBP only has two domains) is shown. The double cysteine was altered to eliminate a slight structural difference between DBP and albumin. By eliminating the second cys, the first cys at position 74 in SFP2 does not pair up with one of the other cys and can be used to carry small molecules, as is done with albumin.

The second change eliminates an O glycosylation site that is heterogeneously glycosylated in vivo.

(SEQ ID NO: 5 is SFP2; SEQ ID NO: 7 is DBP)

Domain 1 and 2 of SFP2 aligned with DBP

```

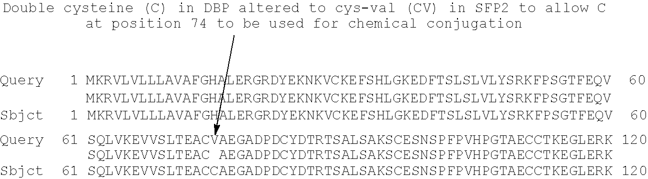

Query 121 LCMAALKHQPQEFPTYVEPTNDEICEAFRKDPKEYANQFMWEYSTNYGQAPLSLLVSYTK 180
                    LCMAALKHQPQEFPTYVEPTNDEICEAFRKDPKEYANQFMWEYSTNYGQAPLSLLVSYTK
Sbjct 121 LCMAALKHQPQEFPTYVEPTNDEICEAFRKDPKEYANQFMWEYSTNYGQAPLSLLVSYTK 180
Query 181 SYLSMVGSCCTSASPTVCFLKERLQLKHLSLLTTLSNRVCSQYAAYGEKKSRLSNLIKLA 240
                    SYLSMVGSCCTSASPTVCFLKERLQLKHLSLLTTLSNRVCSQYAAYGEKKSRLSNLIKLA
Sbjct 181 SYLSMVGSCCTSASPTVCFLKERLQLKHLSLLTTLSNRVCSQYAAYGEKKSRLSNLIKLA 240

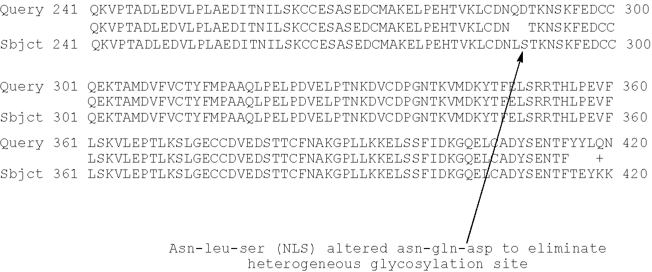

```

Preferred polypeptide fragments of the invention are fragments comprising, or alternatively, consisting of, an amino acid sequence that displays a therapeutic activity and/or functional activity (e.g. biological activity) of the polypeptide sequence of the protein of interest or SFP, which the amino acid sequence is a fragment. Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

### Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant” refers to a protein of interest, or the synthetic fusion protein, which differs in sequence from the protein of interest and/or the albumin superfamily protein portion, but retains at least one functional and/or therapeutic property thereof (e.g., a therapeutic activity and/or biological activity of one of the domains from which the SFP-POI was derived) as described elsewhere herein or otherwise known in the art. Generally, variants are overall very similar, and, in many regions, identical to the amino acid sequence of the protein of interest or albumin superfamily protein.

The present invention is also directed to proteins which comprise, or alternatively consist of, an amino acid sequence which is at least 80%, 85%, 90%, 95%, 96%, 97%, 98%, 99% or 100%, identical to, for example, the amino acid sequence of the SFP itself, the protein of interest, or the SFP-POI. Fragments of these polypeptides are also provided (e.g., those fragments described herein). Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6 times sodium chloride/sodium citrate (SSC) at about 45 degrees Celsius, followed by one or more washes in 0.2 times SSC, 0.1% SDS at about 50-65 degrees Celsius), under highly stringent conditions (e.g., hybridization to filter bound DNA in 6 times sodium chloride/sodium citrate (SSC) at about 45 degrees Celsius, followed by one or more washes in 0.1 times SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequence of an SFP of the invention or a fragment, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The variant will usually have at least 75% (preferably at least about 80%, 90%, 95% or 99%) sequence identity with a length of normal HA or Therapeutic protein which is the same length as the variant. Homology or identity at the nucleotide or amino acid sequence level is determined by BLAST (Basic Local Alignment Search Tool) analysis using the algorithm employed by the programs blastp, blastn, blastx, tblastn and tblastx (Karlin et al., Proc. Natl. Acad. Sci. USA 87: 2264-2268 (1990) and Altschul, J. Mol. Evol. 36: 290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters may be adjusted as follows: Q=10 (gap creation penalty); R=10 (gap extension penalty); wink=1 (generates word hits at every wink.sup.th position along the query); and gapw=16. The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The polynucleotide variants of the invention may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, polypeptide variants in which less than 50, less than 40, less than 30, less than 20, less than 10, or 5-50, 5-25, 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host, such as, yeast or E. coli).

In a preferred embodiment, a polynucleotide encoding SFP of the invention is optimized for expression in yeast or mammalian cells. In further preferred embodiment, a polynucleotide encoding a protein of interest portion of SFP-POI of the invention is optimized for expression in yeast or mammalian cells. In a still further preferred embodiment, a polynucleotide encoding an SFP-POI of the invention is optimized for expression in yeast or mammalian cells.

In an alternative embodiment, a codon optimized polynucleotide encoding a protein of interest portion of an SFP-POI of the invention does not hybridize to the wild type polynucleotide encoding the protein of interest under stringent hybridization conditions as described herein. In a further embodiment, a codon optimized polynucleotide encoding an SFP of the invention does not hybridize to the wild type polynucleotide encoding the albumin superfamily protein under stringent hybridization conditions as described herein. In another embodiment, a codon optimized polynucleotide encoding an SFP of the invention does not hybridize to the wild type polynucleotide encoding the protein of interest portion or the SFP under stringent hybridization conditions as described herein.

In an additional embodiment, polynucleotides encoding a protein of interest portion of SFP-POI of the invention do not comprise, or alternatively consist of, the naturally occurring sequence of that protein of interest. In a further embodiment, polynucleotides encoding an albumin superfamily protein portion of an SFP of the invention do not comprise, or alternatively consist of, the naturally occurring sequence of albumin superfamily protein. In an alternative embodiment, polynucleotides encoding an SFP-POI of the invention do not comprise, or alternatively consist of, the naturally occurring sequence of a protein of interest portion or the SFP.

Naturally occurring variants are called “allelic variants,” and refer to one of several alternate forms of a gene occupying a given locus on a chromosome of an organism. (Genes II, Lewin, B., ed., John Wiley & Sons, New York (1985)). These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the polypeptide of the present invention without substantial loss of biological function. As an example, Ron et al. (J. Biol. Chem. 268: 2984-2988 (1993)) reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less thaw the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants that have a functional activity (e.g., biological activity and/or therapeutic activity). In preferred embodiments the invention provides variants of SFPs that have a functional activity that corresponds to one or more biological and/or therapeutic activities of the protein of interest. Such variants include deletions, insertions, inversions, repeats, and substitutions selected according to general rules known in the art so as have little effect on activity.

In preferred embodiments, the variants of the invention have conservative substitutions. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. See Cunningham and Wells, Science 244:1081-1085 (1989). The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly. Besides conservative amino acid substitution, variants of the present invention include (i) polypeptides containing substitutions of one or more of the non-conserved amino acid residues, where the substituted amino acid residues may or may not be one encoded by the genetic code, or (ii) polypeptides containing substitutions of one or more of the amino acid residues having a substituent group, or (iii) polypeptides which have been fused with or chemically conjugated to another compound, such as a compound to increase the stability and/or solubility of the polypeptide (for example, polyethylene glycol), (iv) polypeptide containing additional amino acids, such as, for example, an IgG Fc fusion region peptide, Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

In specific embodiments, the polypeptides of the invention comprise, or alternatively, consist of, fragments or variants of the amino acid sequence of a SFP-POI, protein of interest alone, or SFP alone, wherein the fragments or variants have 1-5, 5-10, 5-25, 5-50, 10-50 or 50-150, amino acid residue additions, substitutions, and/or deletions when compared to the reference amino acid sequence. In preferred embodiments, the amino acid substitutions are conservative. Nucleic acids encoding these polypeptides are also encompassed by the invention.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York; pgs. 1-12 (1983); Seifter et al., Meth. Enzymol. 182:626-646 (1990); Rattan et al., Ann. N.Y. Acad. Sci. 663:48-62 (1992)).

### Functional Activity

“A polypeptide having functional activity” refers to a polypeptide capable of displaying one or more known functional activities associated with the full-length, pro-protein, and/or mature form of a therapeutic protein. Such functional activities include, but are not limited to, biological activity, antigenicity [ability to bind (or compete with a polypeptide for binding) to an anti-polypeptide antibody], immunogenicity (ability to generate antibody which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a molecule or protein of interest of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention). In preferred embodiments, an SFP of the invention has at least one biological and/or therapeutic activity associated with the protein of interest (or fragment or variant thereof) when it is not fused to albumin.

For example, in one embodiment where one is assaying for the ability of an SFP-POI of the invention to bind or compete with a therapeutic protein for binding to anti-therapeutic polypeptide antibody and/or anti-albumin antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In a preferred embodiment, where a binding partner (e.g., a receptor or a ligand) of a protein of interest is identified, binding to that binding partner by an SFP-POI containing that protein of interest portion of the fusion can be assayed, e.g., by means well-known in the art, such as, for example, reducing and non-reducing gel chromatography, protein affinity chromatography, and affinity blotting. See generally, Phizicky et al., Microbiol. Rev. 59:94-123 (1995). In another embodiment, the ability of physiological correlates of an SFP-POI of the present invention to bind to a substrate(s) of the protein of interest can be routinely assayed using techniques known in the art.

In an alternative embodiment, where the ability of an SFP-POI of the invention to multimerize is being evaluated, association with other components of the multimer can be assayed, e.g., by means well-known in the art, such as, for example, reducing and non-reducing gel chromatography, protein affinity chromatography, and affinity blotting. See generally, Phizicky et al., supra. In addition, assays described herein and otherwise known in the art may routinely be applied to measure the ability of SFP-POIs of the present invention and fragments, variants and derivatives thereof to elicit biological activity and/or therapeutic activity (either in vitro or in vivo) related to either the therapeutic protein portion and/or albumin superfamily portion (SFP) of the present invention. Other methods will be known to the skilled artisan and are within the scope of the invention.

### Albumin Superfamily Members

As described above, the SFP-POI of the invention comprises at least a fragment or variant of a protein of interest and at least a fragment or variant of two or more domains from members of the human albumin superfamily. The two or more domains are associated with each other, preferably by genetic fusion or chemical conjugation. There can be two, three, four, or more different domains that make up the albumin superfamily protein portion of the SFP. By “albumin superfamily member” is meant either a full protein from a member of the albumin superfamily, or a fragment or variant thereof, or a fusion of two or more domains from one or more members of the albumin superfamily. In other words, when the term “albumin superfamily protein” is used, this refers generally to a polypeptide whose individual parts are obtained from one or more albumin superfamily proteins, meaning albumin, alpha-fetoprotein, vitamin D-binding protein, or afamin.

The terms human albumin superfamily, albumin-like superfamily, and albumin superfamily are used interchangeably herein. A number of serum transport proteins are known to be evolutionarily related, including albumin, alpha-fetoprotein, vitamin D-binding protein and afamin [PubMed2481749, PubMed2423133, PubMed7517938]. Albumin is the main protein of plasma; it binds water, cations (such as Ca2+, Na+ and K+), fatty acids, hormones, bilirubin and drugs—its main function is to regulate the colloidal osmotic pressure of blood. Alphafetoprotein (alpha-fetoglobulin) is a foetal plasma protein that binds various cations, fatty acids and bilirubin. Vitamin D-binding protein binds to vitamin D and its metabolites, as well as to fatty acids. The biological role of afamin (alpha-albumin) has not yet been characterized. Proteins from humans, as well as other species, are contemplated herein.

As used herein, a portion of a protein from the albumin superfamily sufficient to prolong the therapeutic activity or shelf-life of the protein of interest refers to a portion of the protein sufficient in length or structure to stabilize or prolong the therapeutic activity of the protein so that the shelf life of the protein of interest portion of the SFP-POI is prolonged or extended compared to the shelf-life in the non-fusion state. This can include the full length protein from the albumin superfamily, or may include one or more fragments thereof that are capable of stabilizing or prolonging the therapeutic activity. Such fragments may be of 10 or more amino acids in length or may include about 15, 20, 25, 30, 50, or more contiguous amino acids. This can include the entire protein selected from the albumin superfamily, or various domains in various combinations from different proteins in the albumin superfamily.

The albumin superfamily protein portion of the SFP-POI of the invention may be a variant of the normal protein. The term “variants” includes insertions, deletions and substitutions, either conservative or non-conservative, where such changes do not substantially alter one or more of the oncotic, useful ligand-binding and non-immunogenic properties of protein itself, or the active site, or active domain which confers the therapeutic activities.

In one example, the SFP can be altered so that it can maximally bind the neonatal Fc protein. The neonatal Fc receptor plays a role in adult salvage of IgG through its occurrence in the pathway of endocytosis in endothelial cells. Fc receptors in the acidic endosomes bind to IgG internalized through pinocytosis, recycling it to the cell surface, releasing it at the basic pH of blood, thereby preventing it from undergoing lysosomal degradation. This mechanism may provide an explanation for the greater half-life of IgG in the blood compared to other isotypes. It has been shown that conjugation of some drugs to the Fc domain of IgG significantly increases their half-life.

The SFP can also be altered to prevent glycosylation.

In particular, the SFP of the invention may include naturally occurring polymorphic variants. The protein may be derived from any vertebrate, especially any mammal, for example human, cow, sheep, or pig. Non-mammalian albumins, for example, include, but are not limited to, hen and salmon. The SFP may be from a different animal than the protein of interest portion.

Generally speaking, the albumin superfamily protein fragment or variant will be at least 100 amino acids long, preferably at least 150 amino acids long. Preferably, the SFP of the invention can comprise at least one subdomain or domain of the albumin superfamily protein, or conservative modifications thereof. If the fusion is based on subdomains, some or all of the adjacent linker is preferably used to link to the protein of interest moiety.

### Synthetic Fusion Proteins

The present invention relates generally to SFPs and methods of treating, preventing, or ameliorating diseases or disorders. As used herein, “synthetic fusion protein” refers to a molecule selected from the human albumin superfamily (or fragments or variants thereof). An SFP comprises at least a fragment or variant of two or more domains selected from members of the albumin superfamily proteins, which are associated with one another, preferably by genetic fusion (i.e., the SFP is generated by translation of a nucleic acid in which a polynucleotide encoding all or a portion of domains from various albumin superfamily members which have also been fused together) or chemical conjugation to one another.

Preferably, the SFP-POI can comprise the SFP/albumin superfamily member as the N-terminal portion, and a protein of interest as the C-terminal portion. Alternatively, an SFP-POI comprising a member of the SFP/albumin superfamily as the C-terminal portion, and a protein of interest as the N-terminal portion may also be used.

In other embodiments, the SFP has a protein of interest fused to both the N-terminus and the C-terminus of the SFP. In a preferred embodiment, the proteins of interest are fused at the N- and C-termini are the same proteins. In a preferred embodiment, the proteins fused at the N- and C-termini are different proteins. In another preferred embodiment, the proteins fused at the N- and C-termini are different proteins, which may be used to treat or prevent the same disease, disorder, or condition. In another preferred embodiment, the proteins of interest fused at the N- and C-termini are different proteins, which may be used to treat or prevent diseases or disorders which are known in the art to commonly occur in patients simultaneously.

As an alternative to the fusion of known therapeutic molecules, the peptides could be obtained by screening libraries constructed as fusions to the N-, C- or N- and C-termini of the SFP, or domain fragment of the same, of typically 6, 8, 12, 20 or 25 or Xn (where X is an amino acid (aa) and in equals the number of residues) randomized amino acids, and in which all possible combinations of amino acids were represented. A particular advantage of this approach is that the peptides may be selected in situ on the albumin superfamily molecule and the properties of the peptide would therefore be as selected for rather than, potentially, modified as might be the case for a peptide derived by any other method then being attached to the protein.

Additionally, the SFP-POI of the invention may include a linker peptide between the fused portions to provide greater physical separation between the moieties and thus maximize the accessibility of the protein of interest portion, for instance, for binding to its cognate receptor. The linker peptide may consist of amino acids such that it is flexible or more rigid.

The linker sequence may be cleavable by a protease or chemically to yield the growth hormone related moiety. Preferably, the protease is one which is produced naturally by the host, for example the S. cerevisiae protease kex2 or equivalent proteases. Therefore, as described above, the SFPs of the invention may have the following formula R1-L-R2; R2-L-R1; or R1-L-R2-L-R1, wherein R1 is at least one protein of interest, peptide or polypeptide sequence, and not necessarily the same protein of interest, L is a linker and R2 is a derived from the albumin superfamily proteins discussed above.

In preferred embodiments, SFP-POI of the invention comprising a protein of interest have extended shelf life, or half-life, compared to the shelf life the same protein when not fused to an albumin superfamily protein. Shelf-life, or half life, typically refers to the time period over which the therapeutic activity of a protein in solution or in some other storage formulation, is stable without undue loss of therapeutic activity. Many of the therapeutic proteins are highly labile in their unfused state. As described below, the typical shelf-life of these therapeutic proteins is markedly prolonged upon incorporation into the SFP of the invention. This half-life can be 5, 10, 15, 20, 25, 30, 35, 40, 45, 50, 55, 60, 65, 70, 75, 80, 85, 90, 95, 100, 150, 200, 250, or 300% or more greater for the protein of interest fused to a albumin superfamily protein compared to the native protein of interest. The half-life can also be increased by 5, 10, 15, 20, 25, 30, 35, 40, 45, 50, 55, 60, 65, 70, 75, 80, 85, 90, 95, 100, 150, 200, 250, or 300% when compared to a protein of interest fused to albumin.

SFPs and SFP-POIs of the invention with “prolonged” or “extended” half-life exhibit greater therapeutic activity relative to a standard that has been subjected to the same storage and handling conditions. The standard may be the unfused full-length protein. When the therapeutic protein portion of the SFP-POI is an analog, a variant, or is otherwise altered or does not include the complete sequence for that protein, the prolongation of therapeutic activity may alternatively be compared to the unfused equivalent of that analog, variant, altered peptide or incomplete sequence. As an example, an SFP-POI of the invention may retain greater than about 100% of the therapeutic activity, or greater than about 105%, 110%, 120%, 130%, 150% or 200% of the therapeutic activity of a standard when subjected to the same storage and handling conditions as the standard when compared at a given time point.

Shelf-life may also be assessed in terms of therapeutic activity remaining after storage, normalized to therapeutic activity when storage began. SFPs and SFP-POIs of the invention with prolonged or extended shelf-life as exhibited by prolonged or extended therapeutic activity may retain greater than about 50% of the therapeutic activity, about 60%, 70%, 80%, or 90% or more of the therapeutic activity of the equivalent unfused therapeutic protein when subjected to the same conditions.

Disclosed is a method of modulating distribution of a peptide of interest within a subject, the method comprising administering to the subject an SFP-POI, wherein the albumin superfamily protein/SFP portion modulates distribution of the peptide of interest within the subject. It can be slower or faster than the distribution of the peptide of interest without the SFP. For example, the administration of the composition to the subject can result in a blood level half-life of the peptide of interest which is greater than the blood level half-life obtained upon administration of the peptide of interest alone, or when the peptide of interest is fused to albumin rather than an albumin superfamily protein fusion.

### Expression of Fusion Proteins

The SFPs and SFP-POIs of the invention may be produced as recombinant molecules by secretion from yeast, a microorganism such as a bacterium, or a human or animal cell line. Preferably, the polypeptide is secreted from the host cells. For example, by fusing the hGH coding sequence to the albumin superfamily coding sequence, either to the 5′ end or 3′ end, it is possible to secrete the SFP and SFP-POIs from yeast without the requirement for a yeast-derived pro sequence.

Hence, a particular embodiment of the invention comprises a DNA construct encoding a signal sequence effective for directing secretion in yeast, particularly a yeast-derived signal sequence (especially one which is homologous to the yeast host), and the fused molecule of the first aspect of the invention, there being no yeast-derived pro sequence between the signal and the mature polypeptide. The Saccharomyces cerevisiae invertase signal is a preferred example of yeast-derived signal sequence.

The present invention also includes a cell, transformed to express an SFP or SFP-POI of the invention. In addition to the transformed host cells themselves, the present invention also contemplates a culture of those cells, including a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris, filamentous fungi (for example Aspergillus), plant cells, animal cells and insect cells.

Preferred yeast strains to be used in the production of SFPs are D88, DXY1 and BXP10. D88 [leu2-3, leu2-122, can1, pra1, ubc4] is a derivative of parent strain AH22his.+ (also known as DB1; see, e.g., Sleep et al. Biotechnology 8:42-46 (1990)). The strain contains a leu2 mutation which allows for auxotropic selection of 2 micron-based plasmids that contain the LEU2 gene. D88 also exhibits a derepression of PRB1 in glucose excess. The PRB I promoter is normally controlled by two checkpoints that monitor glucose levels and growth stage. The promoter is activated in wild type yeast upon glucose depletion and entry into stationary phase. Strain D88 exhibits the repression by glucose but maintains the induction upon entry into stationary phase. The PRA1 gene encodes a yeast vacuolar protease, YscA endoprotease A, that is localized in the ER. The UBC4 gene is in the ubiquitination pathway and is involved in targeting short lived and abnormal proteins for ubiquitin dependant degradation. Isolation of this ubc4 mutation was found to increase the copy number of an expression plasmid in the cell and cause an increased level of expression of a desired protein expressed from the plasmid (see, e.g., International Publication No. WO99/00504, hereby incorporated in its entirety by reference herein).

DXY1, a derivative of D88, has the following genotype: [leu2-3, leu2-122, can1, pra1, ubc4, ura3::yap3]. In addition to the mutations isolated in D88, this strain also has a knockout of the YAPS protease. This protease causes cleavage of mostly di-basic residues (RR, RK, KR, KK) but can also promote cleavage at single basic residues in proteins. Isolation of this yap3 mutation resulted in higher levels of full length HSA production (see, e.g., U.S. Pat. No. 5,965,386, and Kerry-Williams et al., Yeast 14:161-169 (1998), hereby incorporated in their entireties by reference herein).

BXP10 has the following genotype: leu2-3, leu2-122, can1, pra1, ubc4, ura3, yap3::URA3, lys2, hsp150::LYS2, pmt1::URA3. In addition to the mutations isolated in DXY1, this strain also has a knockout of the PMT1 gene and the HSP150 gene. The PMT1 gene is a member of the evolutionarily conserved family of dolichyl-phosphate-D-mannose protein O-mannosyltransferases (Pmts). The transmembrane topology of Pmtlp suggests that it is an integral membrane protein of the endoplasmic reticulum with a role in O-linked glycosylation. This mutation serves to reduce/eliminate O-linked glycosylation of HSA fusions (see, e.g., International Publication No. WO00/44772, hereby incorporated in its entirety by reference herein). Studies revealed that the Hsp150 protein is inefficiently separated from rHA by ion exchange chromatography. The mutation in the HSP150 gene removes a potential contaminant that has proven difficult to remove by standard purification techniques. See, e.g., U.S. Pat. No. 5,783,423, hereby incorporated in its entirety by reference herein.

The desired protein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, 7RP1, LEU2 and URA3. Plasmids pRS413-416 are Yeast Centromere plasmids (Ycps).

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, enzymes that remove protruding, y-single-stranded termini with their 3′ 5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces teiiuini compatible with those of the DNA segment.

Synthetic linkers containing a variety of restriction endonuclease sites are commercially available from a number of sources including International Biotechnologies Inc, New Haven, Conn., USA.

A desirable way to modify the DNA in accordance with the invention, since the superfamily protein is made up of domain from various proteins, is to use the polymerase chain reaction as disclosed by Saiki et al. (1988) Science 239, 487-491. In this method the DNA to be enzymatically amplified is flanked by two specific oligonucleotide primers which themselves become incorporated into the amplified DNA. The specific primers may contain restriction endonuclease recognition sites which can be used for cloning into expression vectors using methods known in the art.

Exemplary genera of yeast contemplated to be useful in the practice of the present invention as hosts for expressing the SFPs or SFP-POIs are Pichia (formerly classified as Hansenula), Saccharomyces, Kluyveromyces, Aspergillus, Candida, Torulopsis, Torulaspora, Schizosaccharomyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, sporidiobolus, Endomycopsis, and the like. Preferred genera are those selected from the group consisting of Saccharomyces, Schizosaccharomyces, Kluyveromyces, Pichia and Torulaspora. Examples of Saccharomyces spp. are S. cerevisiae, S. italicus and S. rouxii.

Examples of Kluyveromyces spp. are K. fragilis, K. lactis and K. marxianus. A suitable Torulaspora species is T. delbrueckii. Examples of Pichia (Hansenula) spp. are P. angusta (formerly H. polymorpha), P. anomala (formerly H. anomala) and P. pastoris. Methods for the transformation of S. cerevisiae are taught generally in EP 251 744, EP 258 067 and WO 90/01063, all of which are incorporated herein by reference.

Preferred exemplary species of Saccharomyces include S. cerevisiae, S. italicus, S. diastaticus, and Zygosaccharomyces rouxii. Preferred exemplary species of Kluyveromyces include K. fragilis and K. lactis. Preferred exemplary species of Hansenula include H. polymorpha (now Pichia angusta), H. anomala (now Pichia anomala), and Pichia capsulata. Additional preferred exemplary species of Pichia include P. pastoris. Preferred exemplary species of Aspergillus include A. niger and A. nidulans. Preferred exemplary species of Yarrowia include Y. lipolytica. Many preferred yeast species are available from the ATCC. For example, the following preferred yeast species are available from the ATCC and are useful in the expression of SFPs: Saccharomyces cerevisiae Hansen, teleomorph strain BY4743 yap3 mutant (ATCC Accession No. 4022731); Saccharomyces cerevisiae Hansen, teleomorph strain BY4743 hsp150 mutant (ATCC Accession No. 4021266); Saccharomyces cerevisiae Hansen, teleomorph strain BY4743 pmt1 mutant (ATCC Accession No. 4023792); Saccharomyces cerevisiae Hansen, teleomorph (ATCC Accession Nos. 20626; 44773; 44774; and 62995); Saccharomyces diastaticus Andrews et Gilliland ex van der Walt, teleomorph (ATCC Accession No. 62987); Kluyveromyces lactis (Dombrowski) van der Walt, teleomorph (ATCC Accession No. 76492); Pichia angusta (Teunisson et al.) Kurtzman, teleomorph deposited as Hansenula polymorpha de Morais et Maia, teleomorph (ATCC Accession No. 26012); Aspergillus niger van Tieghem, anamorph (ATCC Accession No. 9029); Aspergillus niger van Tieghem, anamorph (ATCC Accession No. 16404); Aspergillus nidulans (Eidam) Winter, anamorph (ATCC Accession No. 48756); and Yarrowia lipolytica (Wickerham et al.) van der Walt et von Arx, teleomorph (ATCC Accession No. 201847).

Suitable promoters for S. cerevisiae include those associated with the PGKI gene, GAL1 or GAL10 genes, CYCI, PHO5, TRPI, ADHI, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone, [a mating factor pheromone], the PRBI promoter, the GUT2 promoter, the GPDI promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbpl gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOX1 and AOX2. Gleeson et al. (1986) J. Gen. Microbiol. 132, 3459-3465 include information on Hansenula vectors and transformation, suitable promoters being MOX1 and FMD1; whilst EP 361 991, Fleer et al. (1991) and other-publications from Rhone-Poulenc Rorer teach how to express foreign proteins in Kluyveromyces spp., a suitable promoter being PGKI.

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is preferred.

The desired SFP or SFP-POI may be initially expressed with a secretion leader sequence, which may be any leader effective in the yeast chosen. Leaders useful in S. cerevisiae include that from the mating factor .alpha. polypeptide (MF-1) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MF.alpha.-1, 0 glucanase (BCL2) and killer toxin; S. diastaticus glucoamylase II; S. carlsbergensis.alpha.-galactosidase (MEL1); K. lactis killer toxin; and Candida glucoamylase.

### Additional Methods of Recombinant and Synthetic Production of Synthetic Fusion Proteins (SFPs) and SFP-POIs

The present invention also relates to vectors containing a polynucleotide encoding an SFP and/or SFP-POI of the present invention, host cells, and the production of SFPs and SFP-POI by synthetic and recombinant techniques. The vector may be, for example, a phage, plasmid, viral, or retroviral vector. Retroviral vectors may be replication competent or replication defective. In the latter case, viral propagation generally will occur only in complementing host cells.
The nucleic acids encoding the SFP and SFP-POIs can be incorporated into a specific target in the genome. For example, the target sequence can be the human albumin gene, or alpha-1-antitrypsin, transferrin or antithrombin III, alpha-fetoprotein, or insulin like growth factor II.

The polynucleotides encoding SFPs and SFP-POIs of the invention may be joined to a vector containing a selectable marker for propagation in a host. Generally, a plasmid vector is introduced in a precipitate, such as a calcium phosphate precipitate, or in a complex with a charged-lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS,NSO, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-S1, pPIC3.5K, pPIC9K, and PAO815 (all available from Invitrogen, Carlbad, Calif.). Other suitable vectors will be readily apparent to the skilled artisan.

In one embodiment, polynucleotides encoding the SFP and SFP-POIs of the invention may be fused to signal sequences, which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the SFPs or SFP-POIs of the invention may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides SFPs of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that may be fused to an SFP or SFP-POI of the invention in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA), and a consensus signal sequence (MPTWAWWLFLVLLLALWAPARG). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169 (1992) and in Biblia and Robinson Biotechnol. Prog. 11:1 (1995) which are herein incorporated by reference.

The present invention also relates to host cells containing the above-described vector constructs described herein, and additionally encompasses host cells containing nucleotide sequences of the invention that are operably associated with one or more heterologous control regions (e.g., promoter and/or enhancer) using techniques known of in the art. The host cell can be a higher eukaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Further more, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

In one example, the host cell can be a liver-derived cell, such as a HepG2/C3A cell. The cell can be American Type Culture Collection #CRL-10741.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Therapeutic protein may be replaced with an SFP or SFP-POI corresponding to the Therapeutic protein), and/or to include genetic material (e.g., heterologous polynucleotide sequences such as for example, an SFP or SFP-POI of the invention corresponding to the protein of interest may be included). The genetic material operably associated with the endogenous polynucleotide may activate, alter, and/or amplify endogenous polynucleotides.

In addition, techniques known in the art may be used to operably associate heterologous polynucleotides (e.g., polynucleotides encoding an albumin superfamily protein, or a fragment or variant thereof) and/or heterologous control regions (e.g., promoter and/or enhancer) with endogenous polynucleotide sequences encoding a therapeutic protein via homologous recombination (see, e.g., U.S. Pat. No. 5,641,670, issued Jun. 24, 1997; International Publication Number WO 96/29411; International Publication Number WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties):

SFPs or SFP-POI of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, hydrophobic charge interaction chromatography and lectin chromatography. Most preferably, high performance liquid chromatography (“HPLC”) is employed for purification.

In preferred embodiments the SFP or SFP-POI of the invention are purified using Anion Exchange Chromatography including, but not limited to, chromatography on Q-sepharose, DEAF sepharose, poros HQ, poros DEAE, Toyopearl Q, Toyopearl QAE, Toyopearl DEAE, Resource/Source Q and DEAF, Fractogel Q and DEAE columns.

In specific embodiments the SFP or SFP-POI of the invention are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In specific embodiments the SFP or SFP-POI of the invention are purified using Hydrophobic Interaction Chromatography including, but not limited to, Phenyl, Butyl, Methyl, Octyl, Hexyl-sepharose, poros Phenyl, Butyl, Methyl, Octyl, Hexyl, Toyopearl Phenyl, Butyl, Methyl, Octyl, Hexyl Resource/Source Phenyl, Butyl, Methyl, Octyl, Hexyl, Fractogel Phenyl, Butyl, Methyl, Octyl, Hexyl columns and their equivalents and comparables.

In specific embodiments the SFP or SFP-POI of the invention are purified using Size Exclusion Chromatography including, but not limited to, sepharose S100, S200, S300, superdex resin columns and their equivalents and comparables.

In specific embodiments the SFPs of the invention are purified using Affinity Chromatography including, but not limited to, Mimetic Dye affinity, peptide affinity and antibody affinity columns that are selective for either the HSA or the “fusion target” molecules.

In preferred embodiments SFPs or SFP-POIs of the invention are purified using one or more Chromatography methods listed above. In other preferred embodiments, SFPs of the invention are purified using one or more of the following Chromatography columns, Q sepharose FF column, SP Sepharose FF column, Q Sepharose High Performance Column, Blue Sepharose FF column, Blue Column, Phenyl Sepharose FF column, DEAE Sepharose FF, or Methyl Column.

Additionally, SFPs or SFP-POIs of the invention may be purified using the process described in International Publication No. WO00/44772 which is herein incorporated by reference in its entirety. One of skill in the art could easily modify the process described therein for use in the purification of SFPs of the invention.

SFPs or SFP-POIs of the present invention may be recovered from: products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, SFPs of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express SFPs or SFP-POIs of the invention in a eukaryotic system. Pichia pastoris is a methylotrophic yeast which can metabolize methanol as its sole carbon source. A main step in the methanol metabolization pathway is the oxidation of methanol to formaldehyde using O.sub.2. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O.sub.2. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See Ellis, S. B., et al., Mol. Cell. Biol. 5:1111-21 (1985); Koutz, P. J, et al., Yeast 5:167-77 (1989); Tschopp, J. F., et al. Nucl. Acids Res. 15:3859-76 (1987). Thus, a heterologous coding sequence, such as, for example, a polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding an SFP or SFP-POI of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a polypeptide of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PA0815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a polynucleotide encoding an SFP or SFP-POI of the present invention, may be achieved by cloning the heterologous polynucleotide of the invention into an expression vector such as, for example, pGAPZ or pGAPZalpha, and growing the yeast culture in the absence of methanol.

In addition, SFPs of the invention can be chemically synthesized using techniques known in the art (e.g., see Creighton, 1983, Proteins: Structures and Molecular Principles, W.H. Freeman & Co., N.Y., and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, .alpha.-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Further more, the amino acid can be D (dextrorotary) or L (levorotary).

The invention encompasses SFPs of the present invention which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH.sub.4; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The SFPs may also be modified with a detectable label, such as an enzymatic, fluorescent, isotopic or affinity label to allow for detection and isolation of the protein.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine, carbon, sulfur, tritium, indium, technetium, thallium, gallium, palladium, molybdenum, xenon, and fluorine.

As mentioned, the SFPs or SFP-POIs of the invention may be modified by either natural processes, such as post-translational processing, or by chemical modification techniques which are well known in the art. It will be appreciated that the same type of modification may be present in the same or varying degrees at several sites in a given polypeptide. Polypeptides of the invention may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

SFPS of the invention and antibodies that bind a protein of interest, or fragments or variants thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Further, an SFP of the invention may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxombicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, .beta.-interferon, nerve growth factor, platelet derived growth factor, tissue plasminogen activator, an apoptotic agent, e.g., TNF-alpha, TNF-beta, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to proteins (e.g., SFPs) are well known in the art.

SFP-POIs may also be attached to solid supports, which are particularly useful for immunoassays or purification of polypeptides that are bound by, that bind to, or associate with SFP-POIs of the invention. Such solid supports include, but are not limited to, glass, cellulose, polyacrylamide, nylon, polystyrene, polyvinyl chloride or polypropylene.

SFPs, with or without a therapeutic moiety conjugated to it, administered alone or in combination with cytotoxic factor(s) and/or cytokine(s) can be used as a therapeutic.

Also provided by the invention are chemically modified derivatives of the SFPs of the invention which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The SFPs may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, such as, for example, the method disclosed in EP 0 401 384 (coupling PEG to G-CSF), herein incorporated by reference; see also Malik et al., Exp. Hematol. 20:1028-1035 (1992), reporting pegylation of GM-CSF using tresyl chloride. For example, polyethylene glycol may be covalently bound through amino acid residues via reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the SFPs of the invention may be accomplished by any number of means. For example, polyethylene glycol may be attached to the SFP either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride (CISO.sub.2CH.sub.2CF.sub.3). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in International Publication No. WO 98/32466, the entire disclosure of which is incorporated herein by reference. Pegylated protein products produced using the reaction chemistries set out herein are included within the scope of the invention.

The number of polyethylene glycol moieties attached to each SFP or SFP-POI of the invention (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

The polypeptides of the invention can be recovered and purified from chemical synthesis and recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Well known techniques for refolding protein may be employed to regenerate active conformation when the polypeptide is denatured during isolation and/or purification.

The presence and quantity of SFPs or SFP-POIs of the invention may be determined using ELISA, a well known immunoassay known in the art. In one ELISA protocol that would be useful for detecting/quantifying SFPs or SFP-POIs of the invention, comprises the steps of coating an ELISA plate with an anti-human serum albumin antibody, blocking the plate to prevent non-specific binding, washing the ELISA plate, adding a solution containing the SFP or SFP-POIs of the invention (at one or more different concentrations), adding a secondary anti-therapeutic protein specific antibody coupled to a detectable label (as described herein or otherwise known in the art), and detecting the presence of the secondary antibody. In an alternate version of this protocol, the ELISA plate might be coated with the anti-therapeutic protein specific antibody and the labeled secondary reagent might be the anti-human albumin superfamily specific antibody.

### Uses of the Polynucleotides

Each of the polynucleotides identified herein can be used in numerous ways as reagents. The following description should be considered exemplary and utilizes known techniques.

Certain polynucleotides of the present invention are useful to produce the SFPs or SFP-POIs of the invention. As described in more detail below, polynucleotides of the invention (encoding SFPs or SFP-POIs) may be used in recombinant DNA methods useful in genetic engineering to make cells, cell lines, or tissues that express the SFP or SFP-POI encoded by the polynucleotides encoding SFP or SFP-POI of the invention.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

Each of the polypeptides identified herein can be used in numerous ways. The following description should be considered exemplary and utilizes known techniques.

SFPs of the invention are useful to provide immunological probes for differential identification of the tissue(s) (e.g., immunohistochemistry assays such as, for example, ABC immunoperoxidase (Hsu et al., J. Histochem. Cytochem. 29:577-580 (1981)) or cell type(s) (e.g., immunocytochemistry assays).

SFP-POIs can be used to assay levels of polypeptides in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other methods useful for detecting protein gene expression include immunoassays; such as the enzyme linked immunosorbent assay (ELISA) and the radioimmunoassay (RIA). Suitable assay labels are known in the art.

SFP-POIs of the invention can also be detected in vivo by imaging. Labels or markers for in vivo imaging of protein include those detectable by X-radiography, nuclear magnetic resonance (NMR) or electron spin relaxation (ESR). For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the SFP-POI by labeling of nutrients given to a cell line expressing the SFP-POI of the invention.

An SFP-POI which has been labeled with an appropriate detectable imaging moiety, such as a radioisotope, a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries. The SFP-POI will then preferentially accumulate at locations in the body (e.g., organs, cells, extracellular spaces or matrices) where one or more receptors, ligands or substrates (corresponding to that of the therapeutic protein used to make the SFP-POI of the invention) are located. Alternatively, in the case where the SFP-POI comprises at least a fragment or variant of a therapeutic antibody, the labeled SFP-POI will then preferentially accumulate at the locations in the body (e.g., organs, cells, extracellular spaces or matrices) where the polypeptides/epitopes corresponding to those bound by the therapeutic antibody (used to make the SFP-POI of the invention) are located. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)). The protocols described therein could easily be modified by one of skill in the art for use with the SFPs of the invention.

In one embodiment, the invention provides a method for the specific delivery of SFP-POIs of the invention to cells by administering SFP-POIs of the invention (e.g., polypeptides encoded by polynucleotides encoding SFP-POIs of the invention and/or antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering SFP-POIs of the invention in association with toxins or cytotoxic prodrugs.

By “toxin” is meant one or more compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. “Toxin” also includes a cytostatic or cytocidal agent, a therapeutic agent or a radioactive metal ion, e.g., alpha-emitters; luminescent labels, such as luminol; and fluorescent labels, such as fluorescein and rhodamine, and biotin. In a specific embodiment, the invention provides a method for the specific destruction of cells (e.g., the destruction of tumor cells) by administering polypeptides of the invention or antibodies of the invention in association with the radioisotope.

Techniques known in the art may be applied to label polypeptides of the invention. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

The SFPs and SFP-POIs of the present invention are useful for diagnosis, treatment, prevention and/or prognosis of various disorders in mammals, preferably humans. Such disorders include, but are not limited to, those described herein under the section heading “Biological Activities,” below.

Thus, the invention provides a diagnostic method of a disorder, which involves (a) assaying the expression level of a certain polypeptide in cells or body fluid of an individual using an SFP-POI of the invention; and (b) comparing the assayed polypeptide expression level with a standard polypeptide expression level, whereby an increase or decrease in the assayed polypeptide expression level compared to the standard expression level is indicative of a disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Moreover, SFP-POIs of the present invention can be used to treat or prevent diseases or conditions such as, for example, neural disorders, immune system disorders, muscular disorders, reproductive disorders, gastrointestinal disorders, pulmonary disorders, cardiovascular disorders, renal disorders, proliferative disorders, and/or cancerous diseases and conditions. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B, SOD, catalase, DNA repair proteins), to inhibit the activity of a polypeptide (e.g., an oncogene or tumor supressor), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth inhibition, enhancement of the immune response to proliferative cells or tissues).

In particular, SFP-POIs comprising of at least a fragment or variant of a therapeutic antibody can also be used to treat disease (as described supra, and elsewhere herein). For example, administration of an SFP-POI comprising of at least a fragment or variant of a Therapeutic antibody can bind, and/or neutralize the polypeptide to which the Therapeutic antibody used to make the SFP-POI immunospecifically binds, and/or reduce overproduction of the polypeptide to which the Therapeutic antibody used to make the SFP-POI immunospecifically binds. Similarly, administration of an SFP-POI comprising of at least a fragment or variant of a therapeutic antibody can activate the polypeptide to which the therapeutic antibody used to make the SFP-POI immunospecifically binds, by binding to the polypeptide bound to a membrane (receptor).

At the very least, the SFP-POIs of the invention of the present invention can be used as molecular weight markers on SDS-PAGE gels or on molecular sieve gel filtration columns using methods well known to those of skill in the art. SFP-POIs of the invention can also be used to raise antibodies, which in turn may be used to measure protein expression of the therapeutic protein, albumin superfamily protein, and/or the SFP of the invention from a recombinant cell, as a way of assessing transformation of the host cell, or in a biological sample. Moreover, the SFP-POI of the present invention can be used to test the biological activities described herein.

### Diagnostic Assays

The compounds of the present invention are useful for diagnosis, treatment, prevention and/or prognosis of various disorders in mammals, preferably humans. For a number of disorders, substantially altered (increased or decreased) levels of gene expression can be detected in tissues, cells or bodily fluids (e.g., sera, plasma, urine, semen, synovial fluid or spinal fluid) taken from an individual having such a disorder, relative to a “standard” gene expression level, that is, the expression level in tissues or bodily fluids from an individual not having the disorder. Thus, the invention provides a diagnostic method useful during diagnosis of a disorder, which involves measuring the expression level of the gene encoding a polypeptide in tissues, cells or body fluid from an individual and comparing the measured gene expression level with a standard gene expression level, whereby an increase or decrease in the gene expression level(s) compared to the standard is indicative of a disorder. These diagnostic assays may be performed in vivo or in vitro, such as, for example, on blood samples, biopsy tissue or autopsy tissue.

The present invention is also useful as a prognostic indicator, whereby patients exhibiting enhanced or depressed gene expression will experience a worse clinical outcome.

By “assaying the expression level of the gene encoding a polypeptide” is intended qualitatively or quantitatively measuring or estimating the level of a particular polypeptide or the level of the mRNA encoding the polypeptide of the invention in a first biological sample either directly (e.g., by determining or estimating absolute protein level or mRNA level) or relatively (e.g., by comparing to the polypeptide level or mRNA level in a second biological sample). Preferably, the polypeptide expression level or mRNA level in the first biological sample is measured or estimated and compared to a standard polypeptide level or mRNA level, the standard being taken from a second biological sample obtained from an individual not having the disorder or being determined by averaging levels from a population of individuals not having the disorder. As will be appreciated in the art, once a standard polypeptide level or mRNA level is known, it can be used repeatedly as a standard for comparison.

By “biological sample” is intended any biological sample obtained from an individual, cell line, tissue culture, or other source containing polypeptides of the invention (including portions thereof) or mRNA. As indicated, biological samples include body fluids (such as sera, plasma, urine, synovial fluid and spinal fluid) and tissue sources found to express the full length or fragments thereof of a polypeptide or mRNA. Methods for obtaining tissue biopsies and body fluids from mammals are well known in the art. Where the biological sample is to include mRNA, a tissue biopsy is the preferred source.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the polypeptides of the invention are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

The present invention also relates to diagnostic assays such as quantitative and diagnostic assays for detecting levels of polypeptides that bind to, are bound by, or associate with SFPs or SFP-POIs of the invention, in a biological sample (e.g., cells and tissues), including determination of normal and abnormal levels of polypeptides. Thus, for instance, a diagnostic assay in accordance with the invention for detecting abnormal expression of polypeptides that bind to, are bound by, or associate SFPs compared to normal control tissue samples may be used to detect the presence of tumors. Assay techniques that can be used to determine levels of a polypeptide that bind to, are bound by, or associate with SFPs or SFP-POIs of the present invention in a sample derived from a host are well-known to those of skill in the art. Such assay methods include radioimmunoassays, competitive-binding assays, Western Blot analysis and ELISA assays. Assaying polypeptide levels in a biological sample can occur using any art-known method.

Assaying polypeptide levels in a biological sample can occur using a variety of techniques. For example, polypeptide expression in tissues can be studied with classical immunohistological methods (Jalkanen et al., J. Cell. Biol. 101:976-985 (1985); Jalkanen, M., et al., J. Cell. Biol. 105:3087-3096 (1987)). Other methods useful for detecting polypeptide gene expression include immunoassays, such as the enzyme linked immunosorbent assay (ELISA) and the radioimmunoassay (RIA).

The tissue or cell type to be analyzed will generally include those which are known, or suspected, to express the gene of interest (such as, for example, cancer). The protein isolation methods employed herein may, for example, be such as those described in Harlow and Lane (Harlow, E. and Lane, D., 1988, “Antibodies: A Laboratory Manual”, Cold Spring Harbor Laboratory Press, Cold Spring Harbor, N.Y.), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the gene.

For example, SFP-POIs may be used to quantitatively or qualitatively detect the presence of polypeptides that bind to, are bound by, or associate with SFP-POIs of the present invention. This can be accomplished, for example, by immunofluorescence techniques employing a fluorescently labeled SFP-POI coupled with light microscopic, flow cytometric, or fluorimetric detection.

In a preferred embodiment, SFP-POIs comprising at least a fragment or variant of an antibody that immunospecifically binds at least a protein of interest disclosed herein (e.g., the therapeutic protein) or otherwise known in the art may be used to quantitatively or qualitatively detect the presence of gene products or conserved variants or peptide fragments thereof. This can be accomplished, for example, by immunofluorescence techniques employing a fluorescently labeled antibody coupled with light microscopic, flow cytometric, or fluorimetric detection.

The SFP-POIs of the present invention may, additionally, be employed histologically, as in immunofluorescence, immunoelectron microscopy or non-immunological assays, for in situ detection of polypeptides that bind to, are bound by, or associate with an SFP-POI of the present invention. In situ detection may be accomplished by removing a histological specimen from a patient, and applying thereto a labeled antibody or polypeptide of the present invention. The SFP-POIs are preferably applied by overlaying the labeled SFP-POIs onto a biological sample. Through the use of such a procedure, it is possible to determine not only the presence of the polypeptides that bind to, are bound by, or associate with SFP-POIs, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays that detect polypeptides that bind to, are bound by, or associate with SFP-POIs will typically comprise incubating a sample, such as a biological fluid, a tissue extract, freshly harvested cells, or lysates of cells which have been incubated in cell culture, in the presence of a detectably labeled antibody capable of binding gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled SFP-POI of the invention. The solid phase support may then be washed with the buffer a second time to remove unbound antibody or polypeptide. Optionally the antibody is subsequently labeled. The amount of bound label on solid support may then be detected by conventional means.

By “solid phase support or carrier” is intended any support capable of binding a polypeptide (e.g., an SFP, SFP-POI, or polypeptide that binds, is bound by, or associates with an SFP or SFP-POI of the invention.) Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a polypeptide. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, such as a vaccine antigen, or will be able to ascertain the same by use of routine experimentation.

The binding activity of a given lot of SFP-POI may be determined according to well known methods. Those skilled in the art will be able to determine operative and optimal assay conditions for each determination by employing routine experimentation.

In addition to assaying polypeptide levels in a biological sample obtained from an individual, polypeptide can also be detected in vivo by imaging. For example, in one embodiment of the invention, SFP-POIs of the invention are used to image diseased or neoplastic cells.

Labels or markers for in vivo imaging of SFP-POIs of the invention include those detectable by X-radiography, NMR, MRI, CAT-scans or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the SFP-POI by labeling of nutrients of a cell line (or bacterial or yeast strain) engineered.

Additionally, SFP-POIs of the invention whose presence can be detected, can be administered. For example, SFP-POIs of the invention labeled with a radio-opaque or other appropriate compound can be administered and visualized in vivo, as discussed, above for labeled antibodies. Further, such polypeptides can be utilized for in vitro diagnostic procedures.

A polypeptide-specific antibody or antibody fragment which has been labeled with an appropriate detectable imaging moiety, such as a radioisotope, a radio-opaque substance, or a material detectable by nuclear magnetic resonance, is introduced (for example, parenterally, subcutaneously or intraperitoneally) into the mammal to be examined for a disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicures. The labeled SFP-POI will then preferentially accumulate at the locations in the body which contain a polypeptide or other substance that binds to, is bound by or associates with an SFP-POI of the present invention. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)).

One of the ways in which an SFP-POI of the present invention can be detectably labeled is by linking the same to a reporter enzyme and using the linked product in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller et al., J. Clin. Pathol. 31:507-520 (1978); Butler, J. E., Meth. Enzymol. 73:482-523 (1981); Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The reporter enzyme which is bound to the antibody will react with an appropriate substrate, preferably a chromogenic substrate, in such a manner as to produce a chemical moiety which can be detected, for example, by spectrophotometric, fluorimetric or by visual means. Reporter enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the reporter enzyme. Detection may also be accomplished by visual comparison of the extent of enzymatic reaction of a substrate in comparison with similarly prepared standards.

SFP-POIs may also be radiolabelled and used in any of a variety of other immunoassays. For example, by radioactively labeling the SFP-POIs, it is possible to the use the SFP-POIs in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by means including, but not limited to, a gamma counter, a scintillation counter, or autoradiography.

It is also possible to label the SFP-POIs with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, ophthaldehyde and fluorescamine.

The SFP-POI can also be detectably labeled using fluorescence emitting metals. These metals can be attached to the antibody using such metal chelating groups as diethylenetriaminepentacetic acid (DTPA) or ethylenediaminetetraacetic acid (EDTA).

The SFP-POIs can also can be detectably labeled by coupling it to a chemiluminescent compound. The presence of the chemiluminescent-tagged SFP-POI is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label SFP-POIs of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### Transgenic Organisms

Transgenic organisms that express the SFP-POIs of the invention are also included in the invention. Transgenic organisms are genetically modified organisms into which recombinant, exogenous or cloned genetic material has been transferred. Such genetic material is often referred to as a transgene. The nucleic acid sequence of the transgene may include one or more transcriptional regulatory sequences and other nucleic acid sequences such as introns, that may be necessary for optimal expression and secretion of the encoded protein. The transgene may be designed to direct the expression of the encoded protein in a manner that facilitates its recovery from the organism or from a product produced by the organism, e.g. from the milk, blood, urine, eggs, hair or seeds of the organism. The transgene may consist of nucleic acid sequences derived from the genome of the same species or of a different species than the species of the target animal. The transgene may be integrated either at a locus of a genome where that particular nucleic acid sequence is not otherwise normally found or at the normal locus for the transgene.

The term “germ cell line transgenic organism” refers to a transgenic organism in which the genetic alteration or genetic information was introduced into a germ line cell, thereby conferring the ability of the transgenic organism to transfer the genetic information to offspring. If such offspring in fact possess some or all of that alteration or genetic information, then they too are transgenic organisms. The alteration or genetic information may be foreign to the species of organism to which the recipient belongs, foreign only to the particular individual recipient, or may be genetic information already possessed by the recipient. In the last case, the altered or introduced gene may be expressed differently than the native gene.

A transgenic organism may be a transgenic animal or a transgenic plant. Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al. (1993) Hypertension 22(4):630-633; Brenin et al. (1997) Surg. Oncol. 6(2)99-110; Tuan (ed.), Recombinant Gene Expression Protocols, Methods in Molecular Biology No. 62, Humana Press (1997)). The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al. (1996) Genetics 143(4):1753-1760); or, are capable of generating a fully human antibody response (McCarthy (1997) The Lancet 349(9049):405).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al. (1997) Mol. Reprod. Dev. 46(4):515-526; Houdebine (1995) Reprod. Nutr. Dev. 35(6):609-617; Petters (1994) Reprod. Fertil. Dev. 6(5):643-645; Schnieke et al. (1997) Science 278(5346):2130-2133; and Amoah (1997) J. Animal Science 75(2):578-585).

To direct the secretion of the transgene-encoded protein of the invention into the milk of transgenic mammals, it may be put under the control of a promoter that is preferentially activated in mammary epithelial cells. Promoters that control the genes encoding milk proteins are preferred, for example the promoter for casein, beta lactoglobulin, whey acid protein, or lactalbumin (see, e.g., DiTullio (1992) BioTechnology 10:74-77; Clark et al. (1989) BioTechnology 7:487-492; Gorton et al. (1987) BioTechnology 5:1183-1187; and Soulier et al. (1992) FEBS Letts. 297:13). The transgenic mammals of choice would produce large volumes of milk and have long lactating periods, for example goats, cows, camels or sheep.

An SFP of the invention can also be expressed in a transgenic plant, e.g. a plant in which the DNA transgene is inserted into the nuclear or plastidic genome. Plant transformation procedures used to introduce foreign nucleic acids into plant cells or protoplasts are known in the art. See, in general, Methods in Enzymology Vol. 153 (“Recombinant DNA Part D”) 1987, Wu and Grossman Eds., Academic Press and European Patent Application EP 693554. Methods for generation of genetically engineered plants are further described in U.S. Pat. No. 5,283,184, U.S. Pat. No. 5,482,852, and European Patent Application EP 693 554, all of which are hereby incorporated by reference.

### Pharmaceutical or Therapeutic Compositions

The SFP-POIs of the invention or formulations thereof may be administered by any conventional method including parenteral (e.g. subcutaneous or intramuscular) injection or intravenous infusion. The treatment may consist of a single dose or a plurality of doses over a period of time.

While it is possible for an SFP-POI of the invention to be administered alone, it is preferable to present it as a pharmaceutical formulation, together with one or more acceptable carriers. The carrier(s) must be “acceptable” in the sense of being compatible with the SFP-POI and not deleterious to the recipients thereof. Typically, the carriers will be water or saline which will be sterile and pyrogen free. SFPs of the invention are particularly well suited to formulation in aqueous carriers such as sterile pyrogen free water, saline or other isotonic solutions because of their extended shelf-life in solution. For instance, pharmaceutical compositions of the invention may be formulated well in advance in aqueous form, for instance, weeks or months or longer time periods before being dispensed.

For example, wherein the therapeutic protein is hGH, EPO, alpha-IFN or beta-IFN, formulations containing SFP-POI may be prepared taking into account the extended shelf-life of the SFP-POI in aqueous formulations. As discussed above, the shelf-life of many of these Therapeutic proteins are markedly increased or prolonged after fusion to an albumin superfamily protein.

In instances where aerosol administration is appropriate, the SFP-POIs of the invention can be formulated as aerosols using standard procedures. The term “aerosol” includes any gas-borne suspended phase of an SFP-POI of the instant invention which is capable of being inhaled into the bronchioles or nasal passages. Specifically, aerosol includes a gas-borne suspension of droplets of an SFP-POI of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., (1992) Pharmacol. Toxicol. Methods 27:143-159.

The formulations of the invention are also typically non-immunogenic, in part, because of the use of the components of the SFP-POI being derived from the proper species. For instance, for human use, both the therapeutic protein and albumin superfamily portions of the SFP-POI will typically be human. In some cases, wherein either component is non human-derived, that component may be humanized by substitution of key amino acids so that specific epitopes appear to the human immune system to be human in nature rather than foreign.

The formulations may conveniently be presented in unit dosage form and may be prepared by any of the methods well known in the art of pharmacy. Such methods include the step of bringing into association the SFP-POI with the carrier that constitutes one or more accessory ingredients. In general the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders. Dosage formulations may contain the Therapeutic protein portion at a lower molar concentration or lower dosage compared to the non-fused standard formulation for the Therapeutic protein given the extended serum half-life exhibited by many of the SFPs of the invention.

As an example, when an SFP-POI of the invention comprises growth hormone as one or more of the therapeutic protein regions, the dosage form can be calculated on the basis of the potency of the SFP-POI relative to the potency of hGH, while taking into account the prolonged serum half-life and shelf-life of the SFP-POI compared to that of native hGH. Growth hormone is typically administered at 0.3 to 30.0 IU/kg/week, for example 0.9 to 12.0 IU/kg/week, given in three or seven divided doses for a year or more. In an SFP-POI consisting of full length HA fused to full length GH, an equivalent dose in terms of units would represent a greater weight of agent but the dosage frequency can be reduced, for example to twice a week, once a week or less.

Formulations or compositions of the invention may be packaged together with, or included in a kit with, instructions or a package insert referring to the extended shelf-life of the SFP-POI component. For instance, such instructions or package inserts may address recommended storage conditions, such as time, temperature and light, taking into account the extended or prolonged shelf-life of the SFP-POIs of the invention. Such instructions or package inserts may also address the particular advantages of the SFP-POIs of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

SFP-POIs of the invention can also be included in nutraceuticals. For instance, certain SFP-POIs of the invention may be administered in natural products, including milk or milk product obtained from a transgenic mammal which expresses SFP-POI. Such compositions can also include plant or plant products obtained from a transgenic plant which expresses the SFP-POI. The SFP-POI can also be provided in powder or tablet form, with or without other known additives, carriers, fillers and diluents. Nutraceuticals are described in Scott Hegenhart, Food Product Design, December 1993.

The invention also provides methods of treatment and/or prevention of diseases or disorders (such as, for example, any one or more of the diseases or disorders disclosed herein) by administration to a subject of an effective amount of an SFP-POI of the invention or a polynucleotide encoding an SFP-POI of the invention in a pharmaceutically acceptable carrier.

The SFP-POI and/or polynucleotide will be formulated and dosed in a fashion consistent with good medical practice, taking into account the clinical condition of the individual patient (especially the side effects of treatment with the SFP-POI and/or polynucleotide alone), the site of delivery, the method of administration, the scheduling of administration, and other factors known to practitioners. The “effective amount” for purposes herein is thus determined by such considerations.

As a general proposition, the total pharmaceutically effective amount of the SFP-POI in administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the SFP-POI is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

SFP-POIs and/or polynucleotides can be are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

SFP-POIs and/or polynucleotides of the invention are also suitably administered by sustained-release systems. Examples of sustained-release SFPs and/or polynucleotides are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. Additional examples of sustained-release SFPs and/or polynucleotides include suitable polymeric materials (such as, for example, semi-permeable polymer matrices in the form of shaped articles, e.g., films, or mirocapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release SFP-POIs and/or polynucleotides also include liposomally entrapped SFPs and/or polynucleotides of the invention (see generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the SFP-POI and/or polynucleotide are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapeutic.

In yet an additional embodiment, the SFP-POIs and/or polynucleotides of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). Other controlled release systems are discussed in the review by Langer (Science 249:1527-1533 (1990)).

For parenteral administration, in one embodiment, the SFP-POI and/or polynucleotide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the therapeutic.

Generally, the formulations are prepared by contacting the SFP-POI and/or polynucleotide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The SF-POIP is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). SFPs and/or polynucleotides generally are placed into a container having a sterile access port, for example, an intravenous solution bag or vial having a stopper pierceable by a hypodermic injection needle.

SFP-POIs and/or polynucleotides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous SFP-POI and/or polynucleotide solution, and the resulting mixture is lyophilized. The infusion solution is prepared by reconstituting the lyophilized SFP-POI and/or polynucleotide using bacteriostatic Water-for-Injection.

In a specific and preferred embodiment, the SFP-POI formulations comprises 0.01 M sodium phosphate, 0.15 mM sodium chloride, 0.16 micromole sodium octanoate/milligram of fusion protein, 15 micrograms/milliliter polysorbate 80, pH 7.2. In another specific and preferred embodiment, the SFP-POI formulations consists 0.01 M sodium phosphate, 0.15 mM sodium chloride, 0.16 micromole sodium octanoate/milligram of fusion protein, 15 micrograms/milliliter polysorbate 80, pH 7.2. The pH and buffer are chosen to match physiological conditions and the salt is added as a tonicifier. Sodium octanoate has been chosen due to its reported ability to increase the thermal stability of the protein in solution. Finally, polysorbate has been added as a generic surfactant, which lowers the surface tension of the solution and lowers non-specific adsorption of the SFP-POI to the container closure system.

The invention also provides a pharmaceutical pack or kit comprising one or more containers filled with one or more of the ingredients of the SFP-POIs and/or polynucleotides of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the SFP-POIs and/or polynucleotides may be employed in conjunction with other therapeutic compounds.

The SFP-POI and/or polynucleotides of the invention may be administered alone or in combination with adjuvants. Adjuvants that may be administered with the SFP-POI and/or polynucleotides of the invention include, but are not limited to, alum, alum plus deoxycholate (ImmunoAg), MTP-PE (Biocine Corp.), QS21 (Genentech, Inc.), BCG (e.g., THERACYSMPL and nonviable preparations of Corynebacterium parvum. In a specific embodiment, SFP-POIs and/or polynucleotides of the invention are administered in combination with alum. In another specific embodiment, SFP-POIs and/or polynucleotides of the invention are administered in combination with QS-21. Further adjuvants that may be administered with the SFP-POIs and/or polynucleotides of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the SFP-POIs and/or polynucleotides of the invention include, but are not limited to, vaccines directed toward protection against MMR (measles, mumps, rubella), polio, varicella, tetanus/diptheria, hepatitis A, hepatitis B, Haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The SFP-POIs and/or polynucleotides of the invention may be administered alone or in combination with other therapeutic agents. SFP-POIs and/or polynucleotide agents that may be administered in combination with the SFP-POIs and/or polynucleotides of the invention, include but not limited to, chemotherapeutic agents, antibiotics, steroidal and non-steroidal anti-inflammatories, conventional immunotherapeutic agents, and/or therapeutic treatments described below. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the SFP-POIs and/or polynucleotides of the invention are administered in combination with an anticoagulant. Anticoagulants that may be administered with the compositions of the invention include, but are not limited to, heparin, low molecular weight heparin, warfarin sodium (e.g., COUMADIN™), dicumarol, 4-hydroxycoumarin, anisindione (e.g., MIRADON™), acenocoumarol (e.g., nicoumalone, SINTHROME™), indan-1,3-dione, phenprocoumon (e.g., MARCUMAR™), ethyl biscoumacetate (e.g., TROMEXANT™), and aspirin. In a specific embodiment, compositions of the invention are administered in combination with heparin and/or warfarin. In another specific embodiment, compositions of the invention are administered in combination with warfarin. In another specific embodiment, compositions of the invention are administered in combination with warfarin and aspirin. In another specific embodiment, compositions of the invention are administered in combination with heparin. In another specific embodiment, compositions of the invention are administered in combination with heparin and aspirin.

In another embodiment, the SFP-POIs and/or polynucleotides of the invention are administered in combination with thrombolytic drugs. Thrombolytic drugs that may be administered with the compositions of the invention include, but are not limited to, plasminogen, lys-plasminogen, alpha2-antiplasmin, streptokinae (e.g., KABIKINASE™), antiresplace (e.g., EMINASET™ tissue plasminogen activator (t-PA, altevase, ACTIVASET™), urokinase (e.g., ABBOKINASE™), sauruplase, (Prourokinase, single chain urokinase), and aminocaproic acid (e.g., AMICAR™). In a specific embodiment, compositions of the invention are administered in combination with tissue plasminogen activator and aspirin.

In another embodiment, the SFP-POIs and/or polynucleotides of the invention are administered in combination with antiplatelet drugs. Antiplatelet drugs that may be administered with the compositions of the invention include, but are not limited to, aspirin, dipyridamole (e.g., PERSANTINET™), and ticlopidine (e.g., TICLIDT™).

In specific embodiments, the use of anti-coagulants, thrombolytic and/or antiplatelet drugs in combination with SFP-POIs and/or polynucleotides of the invention is contemplated for the prevention, diagnosis, and/or treatment of thrombosis, arterial thrombosis, venous thrombosis, thromboembolism, pulmonary embolism, atherosclerosis, myocardial infarction, transient ischemic attack, unstable angina. In specific embodiments, the use of anticoagulants, thrombolytic drugs and/or antiplatelet drugs in combination with SFP-POIs and/or polynucleotides of the invention is contemplated for the prevention of occulsion of saphenous grafts, for reducing the risk of periprocedural thrombosis as might accompany angioplasty procedures, for reducing the risk of stroke in patients with atrial fibrillation including nonrheumatic atrial fibrillation, for reducing the risk of embolism associated with mechanical heart valves and or mitral valves disease. Other uses for the therapeutics of the invention, alone or in combination with antiplatelet, anticoagulant, and/or thrornbolytic drugs, include, but are not limited to, the prevention of occlusions in extracorporeal devices (e.g., intravascular canulas, vascular access shunts in hemodialysis patients, hemodialysis machines, and cardiopulmonary bypass machines).

In certain embodiments, SFP-POIs and/or polynucleotides of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs). Protease inhibitors that may be administered in combination with the SFP-POIs. In a specific embodiment, antiretroviral agents, nucleoside reverse transcriptase inhibitors, non-nucleoside reverse transcriptase inhibitors, and/or protease inhibitors may be used in any combination with SFPs and/or polynucleotides of the invention to treat AIDS and/or to prevent or treat HIV infection.

In a further embodiment, the SFP-POIs and/or polynucleotides of the invention are administered in combination with an antibiotic agent. Antibiotic agents that may be administered with the SFP-POIs and/or polynucleotides of the invention include, but are not limited to, amoxicillin, beta-lactamases, aminoglycosides, beta-lactam (glycopeptide), beta-lactamases, Clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamethoxazole, and vancomycin.

In other embodiments, the SFP-POIs and/or polynucleotides of the invention are administered in combination with immunestimulants. Immunostimulants that transfoituation to the fusogenic state; Trimeris) and T-1249 (a second-generation fusion inhibitor; Trimeris).

In a further embodiment, the SFP-POIs and/or polynucleotides of the invention are administered in combination with an antiviral agent. Antiviral agents that may be administered with the SFP-POIs and/or polynucleotides of the invention include, but are not limited to, acyclovir, ribavirin, amantadine, and remantidine.

In other embodiments, SFP-POIs and/or polynucleotides of the invention may be administered in combination with anti-opportunistic infection agents. In other embodiments, SFP-POIs and/or polynucleotides of the invention are administered in combination with immunosuppressive agents. In an additional embodiment, SFP-POIs and/or polynucleotides of the invention are administered alone or in combination with one or more intravenous immune globulin preparations.

In certain embodiments, the SFP-POIs and/or polynucleotides of the invention are administered alone or in combination with an anti-inflammatory agent. Anti-inflammatory agents that may be administered with the SFP-POIs and/or polynucleotides of the invention include, but are not limited to, corticosteroids (e.g. betamethasone, budesonide, cortisone, dexamethasone, hydrocortisone, methylprednisolone, prednisolone, prednisone, and triamcinolone), nonsteroidal anti-inflammatory drugs (e.g., diclofenac, diflunisal, etodolac, fenoprofen, floctafenine, flurbiprofen, ibuprofen, indomethacin, ketoprofen, meclofenamate, mefenamic acid, meloxicam, nabumetone, naproxen, oxaprozin, phenylbutazone, piroxicam, sulindac, tenoxicam, tiaprofenic acid, and tolmetin.), as well as antihistamines, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In an additional embodiment, the compositions of the invention are administered alone or in combination with an anti-angiogenic agent. Anti-angiogenic agents that may be administered with the compositions of the invention include, but are not limited to, Angiostatin (Entremed, Rockville, Md.), Troponin-1 (Boston Life Sciences, Boston, Mass.), anti-Invasive Factor, retinoic acid and derivatives thereof, paclitaxel (Taxol), Suramin, Tissue Inhibitor of Metalloproteinase-1, Tissue Inhibitor of Metalloproteinase-2, VEGI, Plasminogen Activator Inhibitor-1, Plasminogen Activator Inhibitor-2, and various forms of the lighter “d group” transition metals. Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide-variety of other anti-angiogenic factors may also be utilized within the context of the present invention. Representative examples include, but are not limited to, platelet factor 4; protamine sulphate; sulphated chitin derivatives (prepared from queen crab shells), (Murata et al., Cancer Res. 51:22-26, (1991)); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Bio. Chem. 267:17321-17326, (1992)); Chymostatin (Tomkinson et al., Biochem J. 286:475-480, (1992)); Cyclodextrin Tetradecasulfate; Eponemycin; Camptothecin; Fumagillin (Ingber et al., Nature 348:555-557, (1990)); Gold Sodium Thiomalate (“GST”; Matsubara and Ziff, J. Clin. Invest. 79:1440-1446, (1987)); anticollagenase-serum; alpha2-antiplasmin (Holmes et al., J. Biol. Chem. 262(4):1659-1664, (1987)); Bisantrene (National Cancer Institute); Lobenzarit disodium (N-(2)-carboxyphenyl-4-chloroanthronilic acid disodium or “CCA”; (Takeuchi et al., Agents Actions 36:312-316, (1992)); and metalloproteinase inhibitors such as BB94.

Additional anti-angiogenic factors that may also be utilized within the context of the present invention include Thalidomide, (Celgene, Warren, N.J.); Angiostatic steroid; AGM-1470 (H. Brem and J. Folkman J. Pediatr. Surg. 28:445-51 (1993)); an integrin alpha v beta 3 antagonist (C. Storgard et al., J. Clin. Invest. 103:47-54 (1999)); carboxynaminolmidazole; Carboxyamidotriazole (CAI) (National Cancer Institute, Bethesda, Md.); Conbretastatin A-4 (CA4P) (OXiGENE, Boston, Mass.); Squalamine (Magainin Pharmaceuticals, Plymouth Meeting, Pa.); TNP-470, (Tap Pharmaceuticals, Deerfield, Ill.); ZD-0101 AstraZeneca (London, UK); APRA (CT2584); Benefin, Byrostatin-1 (SC339555); CGP-41251 (PKC 412); CM101; Dexrazoxane (ICRF187); DMXAA; Endostatin; Flavopridiol; Genestein; GTE; ImmTher; Iressa (ZD1839); Octreotide (Somatostatin); Panretin; Penacillamine; Photopoint; PI-88; Prinomastat (AG-3340) Purlytin; Suradista (FCE26644); Tamoxifen (Nolvadex); Tazarotene; Tetrathiomolybdate; Xeloda (Capecitabine); and 5-Fluorouracil.

Anti-angiogenic agents that may be administered in combination with the compounds of the invention may work through a variety of mechanisms including, but not limited to, inhibiting proteolysis of the extracellular matrix, blocking the function of endothelial cell extracellular matrix adhesion molecules, by antagonizing the function of angiogenesis inducers such as growth factors, and inhibiting integrin receptors expressed on proliferating endothelial cells. Examples of anti-angiogenic inhibitors that interfere with extracellular matrix proteolysis and which may be administered in combination with the compositons of the invention include, but are not lmited to, AG-3340 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), and Metastat (Aeterna, St-Foy, Quebec). Examples of anti-angiogenic inhibitors that act by blocking the function of endothelial cell-extracellular matrix adhesion molecules and which may be administered in combination with the compositons of the invention include, but are not lmited to, EMD-121974 (Merck KcgaA Darmstadt, Germany) and Vitaxin (Ixsys, La Jolla, Calif./Medimmune, Gaithersburg, Md.). Examples of anti-angiogenic agents that act by directly antagonizing or inhibiting angiogenesis inducers and which may be administered in combination with the compositons of the invention include, but are not lmited to, Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), and SU-6668 (Sugen). Other anti-angiogenic agents act to indirectly inhibit angiogenesis. Examples of indirect inhibitors of angiogenesis which may be administered in combination with the compositons of the invention include, but are not limited to, IM-862 (Cytran, Kirkland, Wash.), Interferon-alpha, IL-12 (Roche, Nutley, N.J.), and Pentosan polysulfate (Georgetown University, Washington, D.C.).

In particular embodiments, the use of compositions of the invention in combination with anti-angiogenic agents is contemplated for the treatment, prevention, and/or amelioration of an autoimmune disease, such as for example, an autoimmune disease described herein. In a particular embodiment, the use of compositions of the invention in combination with anti-angiogenic agents is contemplated for the treatment, prevention, and/or amelioration of arthritis. In a more particular embodiment, the use of compositions of the invention in combination with anti-angiogenic agents is contemplated for the treatment, prevention, and/or amelioration of rheumatoid arthritis.

In another embodiment, the polynucleotides encoding a polypeptide of the present invention are administered in combination with an angiogenic protein, or polynucleotides encoding an angiogenic protein. Examples of angiogenic proteins that may be administered with the compositions of the invention include, but are not limited to, acidic and basic fibroblast growth factors, VEGF-1, VEGF-2, VEGF-3, epidermal growth factor alpha and beta, platelet-derived endothelial cell growth factor, platelet-derived growth factor, tumor necrosis factor alpha, hepatocyte growth factor, insulin-like growth factor, colony stimulating factor, macrophage colony stimulating factor, granulocyte/macrophage colony stimulating factor, and nitric oxide synthase.

In additional embodiments, compositions of the invention are administered in combination with a chemotherapeutic agent. Chemotherapeutic agents that may be administered with the SFPs and/or polynucleotides of the invention include, but are not limited to alkylating agents such as nitrogen mustards (for example, Mechlorethamine, cyclophosphamide, Cyclophosphamide Ifosfamide, Melphalan (L-sarcolysin), and Chlorambucil), ethylenimines and methylmelamines (for example, Hexamethylmelamine and Thiotepa), alkyl sulfonates (for example, Busulfan), nitrosoureas (for example, Carmustine (BCNU), Lomustine (CCNU), Semustine (methyl-CCNU), and Streptozocin (streptozotocin)), triazenes (for example, Dacarbazine (DTIC; dimethyltriazenoimidazolecarboxamide)), folic acid analogs (for example, Methotrexate (amethopterin)), pyrimidine analogs (for example, Fluorouacil (5-fluorouracil; 5-FU) Floxuridine (fluorodeoxyuridine; FudR), and Cytarabine (cytosine arabinoside)), purine analogs and related inhibitors (for example, Mercaptopurine (6-mercaptopurine; 6-MP), Thioguanine (6-thioguanine; TG), and Pentostatin (2′-deoxycoformycin)), vinca alkaloids (for example, Vinblastine (VLB, vinblastine sulfate)) and Vincristine (vincristine sulfate)), epipodophyllotoxins (for example, Etoposide and Teniposide), antibiotics (for example, Dactinomycin (actinomycin D), Daunorubicin (daunomycin; rubidomycin), Doxorubicin, Bleomycin, Plicamycin (mithramycin), and Mitomycin (mitomycin C), enzymes (for example, L-Asparaginase), biological response modifiers (for example, Interferon-alpha and interferon-alpha-2b), platinum coordination compounds (for example, Cisplatin (cis-DDP) and Carboplatin), anthracenedione (Mitoxantrone), substituted ureas (for example, Hydroxy urea), methylhydrazine derivatives (for example, Procarbazine (N-methyl hydrazine; MIH), adrenocorticosteroids (for example, Prednisone), progestins (for example, Hydroxyprogesterone caproate, Medroxyprogesterone, Medroxyprogesterone acetate, and Megestrol acetate), estrogens (for example, Diethylstilbestrol (DES), Diethylstilbestrol diphosphate, Estradiol, and Ethinyl estradiol), antiestrogens (for example, Tamoxifen), androgens (Testosterone proprionate, and Fluoxymesterone), antiandrogens (for example, Flutamide), gonadotropin-releasing horomone analogs (for example, Leuprolide), other hormones and hormone analogs (for example, methyltestosterone, estramustine, estramustine phosphate sodium, chlorotrianisene, and testolactone), and others (for example, dicarbazine, glutamic acid, and mitotane).

In one embodiment, the compositions of the invention are administered in combination with one or more of the following drugs: inflixirnab (also known as Remicade™ Centocor, Inc.), Trocade (Roche, RO-32-3555), Leflunomide (also known as Arava™ from Hoechst Marion Roussel), Kineret™ (an IL-1 Receptor antagonist also known as Anakinra from Amgen, Inc.)

In a specific embodiment, compositions of the invention are administered in combination with CHOP (cyclophosphamide, doxorubicin, vincristine, and prednisone) or combination of one or more of the components of CHOP. In one embodiment, the compositions of the invention are administered in combination with anti-CD20 antibodies, human monoclonal anti-CD20 antibodies. In another embodiment, the compositions of the invention are administered in combination with anti-CD20 antibodies and CHOP, or anti-CD20 antibodies and any combination of one or more of the components of CHOP, particularly cyclophosphamide and/or prednisone. In a specific embodiment, compositions of the invention are administered in combination with Rituximab. In a further embodiment, compositions of the invention are administered with Rituximab and CHOP, or Rituximab and any combination of one or more of the components of CHOP, particularly cyclophosphamide and/or prednisone. In a specific embodiment, compositions of the invention are administered in combination with tositumomab. In a further embodiment, compositions of the invention are administered with tositumomab and CHOP, or tositumomab and any combination of one or more of the components of CHOP, particularly cyclophosphamide and/or prednisone. The anti-CD20 antibodies may optionally be associated with radioisotopes, toxins or cytotoxic prodrugs.

In another specific embodiment, the compositions of the invention are administered in combination Zevalin™. In a further embodiment, compositions of the invention are administered with Zevalin™ and CHOP, or Zevalin™ and any combination of one or more of the components of CHOP, particularly cyclophosphamide and/or prednisone. Zevalin™may be associated with one or more radisotopes.

In an additional embodiment, the SFP-POIs and/or polynucleotides of the invention are administered in combination with cytokines Cytokines that may be administered with the SFP-POIs and/or polynucleotides of the invention include, but are not limited to, IL2, IL3, IL4, IL5, IL6, IL7, IL10, IL12, IL13, IL15, anti-CD40, CD40L, IFN-gamma and TNF-alpha. In another embodiment, SFP-POIs and/or polynucleotides of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In one embodiment, the SFP-POIs and/or polynucleotides of the invention are administered in combination with members of the TNF family. TNF, TNF-related or TNF-like molecules that may be administered with the SFP-POIs and/or polynucleotides of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TRANK, TR9 (International Publication No. WO 98/56892), TR10 (International Publication No. WO 98/54202), 312C2 (International Publication No. WO 98/06842), and TR12, and soluble fowls CD154, CD70, and CD153.

In an additional embodiment, the SFP-POIs and/or polynucleotides of the invention are administered in combination with angiogenic proteins. Angiogenic proteins that may be administered with the SFP-POIs and/or polynucleotides of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (PlGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B 186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are herein incorporated by reference in their entireties.

In an additional embodiment, the SFP-POIs and/or polynucleotides of the invention are administered in combination with Fibroblast Growth Factors. Fibroblast Growth Factors that may be administered with the SFP-POIs and/or polynucleotides of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In an additional embodiment, the SFP-POIs and/or polynucleotides of the invention are administered in combination with hematopoietic growth factors. Hematopoietic growth factors that may be administered with the SFP-POIs and/or polynucleotides of the invention include, but are not limited to, granulocyte macrophage colony stimulating factor (GM-CSF) (sargramostim, LEUKINE™, PROKINE™), granulocyte colony stimulating factor (G-CSF) (filgrastim, NEUPOGEN™), macrophage colony stimulating factor (M-CSF, CSF-1) erythropoietin (epoetin alfa, EPOGEN™, PROCRIT™), stem cell factor (SCF, c-kit ligand, steel factor), megakaryocyte colony stimulating factor, PIXY321 (a GMCSF/IL-3 fusion protein), interleukins, especially any one or more of IL-1 through IL-12, interferon-gamma, or thrombopoietin.

In certain embodiments, SFP-POIs and/or polynucleotides of the present invention are administered in combination with adrenergic blockers, such as, for example, acebutolol, atenolol, betaxolol, bisoprolol, carteolol, labetalol, metoprolol, nadolol, oxprenolol, penbutolol, pindolol, propranolol, sotalol, and timolol.

In another embodiment, the SFP-POIs and/or polynucleotides of the invention are administered in combination with an antiarrhythmic drug (e.g., adenosine, amidoarone, bretylium, digitalis, digoxin, digitoxin, diliazem, disopyramide, esmolol, flecamide, lidocaine, mexiletine, moricizine, phenyloin, procainamide, N-acetyl procainamide, propafenone, propranolol, quinidine, sotalol, tocamide, and verapamil).

In another embodiment, the SFP-POIs and/or polynucleotides of the invention are administered in combination with diuretic agents, such as carbonic anhydrase-inhibiting agents (e.g., acetazolamide, dichlorphenamide, and methazolamide), osmotic diuretics (e.g., glycerin, isosorbide, mannitol, and urea), diuretics that inhibit Na.sup.+-K.sup.+-2Cl.sup.-symport (e.g., furosemide, bumetanide, azosemide, piretanide, tripamide, ethacrynic acid, muzolimine, and torsemide), thiazide and thiazide-like diuretics (e.g., bendroflumethiazide, benzthiazide, chlorothiazide, hydrochlorothiazide, hydroflumethiazide, methyclothiazide, polythiazide, trichormethiazide, chlorthalidone, indapamide, metolazone, and quinethazone), potassium sparing diuretics (e.g., amiloride and triamterene), and mineralcorticoid receptor antagonists (e.g., spironolactone, canrenone, and potassium canrenoate).

In certain embodiments, the SFP-POIS and/or polynucleotides of the invention are administered in combination with agents used to treat psychiatric disorders. Psychiatric drugs that may be administered with the SFPs and/or polynucleotides of the invention include, but are not limited to, antipsychotic agents (e.g., chlorpromazine, chlorprothixene, clozapine, fluphenazine, haloperidol, loxapine, mesoridazine, molindone, olanzapine, perphenazine, pimozide, quetiapine, risperidone, thioridazine, thiothixene, trifluoperazine, and triflupromazine), antimanic agents (e.g., carbamazepine, divalproex sodium, lithium carbonate, and lithium citrate), antidepressants (e.g., amitriptyline, amoxapine, bupropion, citalopram, clomipramine, desipramine, doxepin, fluvoxamine, fluoxetine, imipramine, isocarboxazid, maprotiline, mirtazapine, nefazodone, nortriptyline, paroxetine, phenelzine, protriptyline, sertraline, tranylcypromine, trazodone, trimipramine, and venlafaxine), antianxiety agents (e.g., alprazolam, buspirone, chlordiazepoxide, clorazepate, diazepam, halazepam, lorazepam, oxazepam, and prazepam), and stimulants (e.g., d-amphetamine, methylphenidate, and pemoline).

In other embodiments, the SFP-POIs and/or polynucleotides of the invention are administered in combination with agents used to treat neurological disorders. Neurological agents that may be administered with the SFPs and/or polynucleotides of the invention include, but are not limited to, antiepileptic agents (e.g., carbamazepine, clonazepam, ethosuximide, phenobarbital, phenyloin, primidone, valproic acid, divalproex sodium, felbamate, gabapentin, lamotrigine, levetiracetam, oxcarbazepine, tiagabine, topiramate, zonisamide, diazepam, lorazepam, and clonazepam), antiparkinsonian agents (e.g., levodopa/carbidopa, selegiline, amantidine, bromocriptine, pergolide, ropinirole, pramipexole, benztropine; biperiden; ethopropazine; procyclidine; trihexyphenidyl, tolcapone), and ALS therapeutics (e.g. riluzole).

In another embodiment, SFP-POIs and/or polynucleotides of the invention are administered in combination with vasodilating agents and/or calcium channel blocking agents. Vasodilating agents that may be administered with the SFP-POIs and/or polynucleotides of the invention include, but are not limited to, Angiotensin Converting Enzyme (ACE) inhibitors (e.g., papaverine, isoxsuprine, benazepril, captopril, cilazapril, enalapril, enalaprilat, fosinopril, lisinopril, moexipril, perindopril, quinapril, ramipril, spirapril, trandolapril, and nylidrin), and nitrates (e.g., isosorbide dinitrate, isosorbide mononitrate, and nitroglycerin). Examples of calcium channel blocking agents that may be administered in combination with the SFP-POIs and/or polynucleotides of the invention include, but are not limited to amlodipine, bepridil, diltiazem, felodipine, flunarizine, isradipine, nicardipine, nifedipine, nimodipine, and verapamil.

In certain embodiments, the SFP-POIs and/or polynucleotides of the invention are administered in combination with treatments for gastrointestinal disorders.

In additional embodiments, the SFP-POIs and/or polynucleotides of the invention are administered in combination with other therapeutic or prophylactic regimens, such as, for example, radiation therapy.

The invention also provides a pharmaceutical pack or kit comprising one or more containers filled with one or more of the ingredients of the pharmaceutical compositions comprising SFPs of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Gene Therapy

Constructs encoding SFP-POIs of the invention can be used as a part of a gene therapy protocol to deliver therapeutically effective doses of the SFP-POI. A preferred approach for in vivo introduction of nucleic acid into a cell is by use of a viral vector containing nucleic acid, encoding an SFP-POI of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous nucleic acid molecules encoding SFP-POIs in vivo. These vectors provide efficient delivery of nucleic acids into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:27 1). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals.

Another viral gene delivery system useful in the present invention uses adenovirus-derived vectors. The genome of an adenovirus can be manipulated such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al., (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., cited supra; Haj-Ahmand et al., J. Virol. 57:267 (1986)).

In another embodiment, non-viral gene delivery systems of the present invention rely on endocytic pathways for the uptake of the subject nucleotide molecule by the targeted cell. Exemplary gene delivery systems of this type include liposomal derived systems, poly-lysine conjugates, and artificial viral envelopes. In a representative embodiment, a nucleic acid molecule encoding an SFP-POI of the invention can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

Gene delivery systems for a gene encoding an SFP-POI of the invention can be introduced into a patient by any of a number of methods. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al. (1994) PNAS 91: 3054-3057). The pharmaceutical preparation of the gene therapy construct can consist essentially of the gene delivery system in an acceptable diluent, or can comprise a slow release matrix in which the gene delivery vehicle is imbedded. Where the SFP-POI can be produced intact from recombinant cells, e.g. retroviral vectors, the pharmaceutical preparation can comprise one or more cells which produce the SFP.

### Additional Gene Therapy Methods

Also encompassed by the invention are gene therapy methods for treating or preventing disorders, diseases and conditions. The gene therapy methods relate to the introduction of nucleic acid (DNA, RNA and antisense DNA or RNA) sequences into an animal to achieve expression of an SFP-POI of the invention. This method requires a polynucleotide which codes for an SFP-POI of the present invention operatively linked to a promoter and any other genetic elements necessary for the expression of the fusion protein by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a polynucleotide encoding an SFP-POI of the present invention ex vivo, with the engineered cells then being provided to a patient to be treated with the fusion protein of the present invention. Such methods are well-known in the art. For example, see Belldegrun, A., et al., J. Natl. Cancer Inst. 85: 207-216 (1993); Ferrantini, M. et al., Cancer Research 53: 1107-1112 (1993); Ferrantini, M. et al., J. Immunology 153: 4604-4615 (1994); Kaido, T., et al., Int. J. Cancer 60: 221-229 (1-995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the polynucleotide constructs can be delivered by any method that delivers injectable materials to the cells of an animal, such as, injection into the interstitial space of tissues (heart, muscle, skin, lung, liver, and the like). The polynucleotide constructs may be delivered in a pharmaceutically acceptable liquid or aqueous carrier.

In one embodiment, polynucleotides encoding the SFP-POIs of the present invention is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, polynucleotides encoding the SFP-POIs of the present invention can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEF1N5, pcDNA3.1, and pRc/CMV2 available from Invitrogen. Other suitable vectors will be readily apparent to the skilled artisan.

Any strong promoter known to those skilled in the art can be used for driving the expression of the polynucleotide sequence. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for the gene corresponding to the therapeutic protein portion of the SFP-POI of the invention.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked nucleic acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Feigner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Feigner et al., Proc. Natl Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Feigner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca.sup.2+-EDTA chelation (Papahadjopoulos et al., Biochim. Biophys. Acta (1975) 394:483; Wilson et al., Cell 17:77 (1979)); ether injection (Deamer, D. and Bangham, A., Biochim. Biophys. Acta 443:629 (1976); Ostro et al., Biochem. Biophys. Res. Commun. 76:836 (1977); Fraley et al., Proc. Natl. Acad. Sci. USA 76:3348 (1979)); detergent dialysis (Enoch, H. and Strittmatter, P., Proc. Natl. Acad. Sci. USA 76:145 (1979)); and reverse-phase evaporation (REV) (Fraley et al., J. Biol. Chem. 255:10431 (1980); Szoka, F. and Papahadjopoulos, D., Proc. Natl. Acad. Sci. USA 75:145 (1978); Schaefer-Ridder et al., Science 215:166 (1982)), which are herein incorporated by reference.

Generally, the ratio of DNA to liposomes will be from about 10:1 to about 1:10. Preferably, the ration will be from about 5:1 to about 1:5. More preferably, the ration will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/9469 provide methods for delivering DNA-cationic lipid complexes to mammals.

In certain embodiments, cells are engineered, ex vivo or in vivo, using a retroviral particle containing RNA which comprises a sequence encoding an SFP-POI of the present invention. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14X, VT-19-17-H2, RCRE, RCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO.sub.4 precipitation. In one alternative, the retroviral plasmid vector may be encapsulated into a liposome, or coupled to a lipid, and then administered to a host.

The producer cell line generates infectious retroviral vector particles which include polynucleotide encoding an SFP-POI of the present invention. Such retroviral vector particles then may be employed, to transduce eukaryotic cells, either in vitro or in vivo. The transduced eukaryotic cells will express a fusion protin of the present invention.

In certain other embodiments, cells are engineered, ex vivo or in vivo, with polynucleotide contained in an adenovirus vector. Adenovirus can be manipulated such that it encodes and expresses fusion protein of the present invention, and at the same time is inactivated in its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz et al. Am. Rev. Respir. Dis. 109:233-238 (1974)). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1a and E1b, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells, either ex vivo or in vivo. The transduced cells will contain the polynucleotide construct integrated into its genome, and will express a fusion protein of the invention.

Another method of gene therapy involves operably associating heterologous control regions and endogenous polynucleotide sequences (e.g. encoding a polypeptide of the present invention) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijistra et al., Nature 342:435-438 (1989), which are herein encorporated by reference. This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous sequence is placed under the control of the promoter. The promoter then drives the expression of the endogenous sequence.

The polynucleotide encoding an SFP-POI of the present invention may contain a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, SFP-POI of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include fusion proteins of the present invention complexed to a targeted delivery vehicle of the present invention. Suitable delivery vehicles for use with systemic administration comprise liposomes comprising ligands for targeting the vehicle to a particular site. In specific embodiments, suitable delivery vehicles for use with systemic administration comprise liposomes comprising SFP-POIs of the invention for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

SFP-POIs of the present invention can be administered to any animal, preferably to mammals and birds. Preferred mammals include humans, dogs, cats, mice, rats, rabbits sheep, cattle, horses and pigs, with humans being particularly preferred.

### Biological Activities

SFP-POIs and/or polynucleotides encoding SFP-POIs of the present invention, can be used in assays to test for one or more biological activities. If an SFP-POI and/or polynucleotide exhibits an activity in a particular assay, it is likely that the therapeutic protein corresponding to the fusion protein may be involved in the diseases associated with the biological activity. Thus, the fusion protein could be used to treat the associated disease.

Members of the secreted family of proteins are believed to be involved in biological activities associated with, for example, cellular signaling. Accordingly, SFP-POIs of the invention and polynucleotides encoding these proteins, may be used in diagnosis, prognosis, prevention and/or treatment of diseases and/or disorders associated with aberrant activity of secreted polypeptides.

In a preferred embodiment, SFP-POIs of the invention comprising a protein of interest portion corresponding to Angiopoietin 1, Chemokine Binding Proteins, Lactoferrin, VEGF-1, ABC1, Acidic FGF-Pseudomonas exotoxin Fusion protein, Calcitonin gene-related peptide, Ectoapyrases, EGF (Epidermal growth factor), Fibrolase, FGF-2, FGF-1, Kistrin, Kunitz protease inhibitor 1 (KPI 1), Leptin, Lys plasminogen, NIF (Neutrophil inhibitory factor), Staphylokinase, TGF Beta 1, Tissue Factor Pathway Inhibitor, t-PA, Urokinase, and/or fragments and/or variants thereof may be used to treat, prevent, diagnose, prognose, and/or detect blood-related disorders or cardiovascular disorders and/or diseases, disorders or conditions as described under “Blood Related Disorders,” “Anti-Angiogenesis Activity,” and/or “Cardiovascular Disorders” infra.

In a preferred embodiment, SFP-POIs of the invention comprising a protein of interest portion corresponding to Adiposin, Angiopoietin 2, Anti-dorsalizing morphogenetic protein-1 (ADMP), APO.sub.2 Ligand (TRAIL), Arresten, BMP-2 (Bone Morphogenetic Protein 2; Bone-related protein), BRCA1 (BRCA1 tumor suppressor protein), BRCA2, Calreticulin, CD40 ligand, Contortrostatin, Decorin, Del-1, EGF (Epideimal growth factor), EMAP II (Endothelial monocyte activating polypeptide II), FLT3 ligand, HCG (Human chorionic gonadotropin), Heat shock protein, interleukins (such as, for example, IL-1 (Interleukin-1), IL-4, IL-10, IL-12), interleukin-toxin chimeras (such as, for example, IL2-diphtheria toxin chimera, IL4-diphtheria toxin chimera, IL6-diphtheria toxin chimera, IL6-Pseudomonas exotoxin chimera), Interferon (such as, for example, interferon gamma, interferon omega), Maspin, Methioninase, MSH-diphtheria toxin chimera, Neutral endopeptidase, Osteoprotegrin, Patched, Progenipoietin, Ranpirnase, Stem cell factor, TGF Beta 1, TGF Beta 2, Tie-2, TNF Alpha, Troponin 1, Viscumin and/or fragments and/or variants thereof may be used to treat, prevent, diagnose, prognose, and/or detect cancers, solid tumors, neoplasms and/or diseases, disorders or conditions as described under “Hyperproliferative Disorders”, “Immune Activity”, and/or “Diseases at the Cellular Level” infra.

In a preferred embodiment, SFP-POIs of the invention comprising a protein of interest portion corresponding to TNF Receptor and/or fragments or variants thereof can be used to treat, prevent, diagnose, prognose, and/or detect Rheumatoid arthritis; Cachexia; Heart failure; HIV 1 infections; Juvenile rheumatoid arthritis; Psoriasis; Psoriatic arthritis; Septic shock; Transplant rejection; allergic asthma, and/or as described under “Immune Acitivity”, “Infectious Disease” and/or “Cardiovascular Disorders” infra.

In a preferred embodiment, SFP-POIs of the invention comprising a protein of interest portion corresponding to Follicle Stimulating Hormone and/or fragments or variants thereof can be used to treat, prevent, diagnose, prognose, and/or detect Female Infertility; Male Infertility, and/or as described under “Endocrine Disorders” and/or “Reproductive System Disorders” infra.

In a preferred embodiment, SFP-POIs of the invention comprising a protein of interest portion corresponding to Human luteinizing hormone and/or fragments or variants thereof can be used to treat, prevent, diagnose, prognose, and/or detect Infertility, and/or as described under “Endocrine Disorders” and/or “Reproductive System Disorders” infra.

In a preferred embodiment, SFP-POIs of the invention comprising a protein of interest portion corresponding to Urokinase and/or fragments or variants thereof can be used for catheter clearence and/or to treat, prevent, diagnose, prognose, and/or detect Coronary restenosis; Diabetic retinopathy; Myocardial infarction; Thrombosis; Vitreous haemorrhage; Peripheral vascular disorders; Stroke and/or as described under “Renal Disorders” and/or “Cardiovascular Disorders” infra.

In a preferred embodiment, SFP-POIs of the invention comprising a protein of interest portion corresponding to B-glucocerebrosidase and/or fragments or variants thereof can be used to treat, prevent, diagnose, prognose, and/or detect Gaucher's disease and/or as described under “Blood Related Disorders” and/or “Hyperproliferative Disorders” infra.

In alternative embodiments, fusion proteins of the present invention may be used in the diagnosis, prognosis, prevention and/or treatment of diseases and/or disorders relating to diseases and disorders of the endocrine system (see, e.g., “Endocrine Disorders” section below), the nervous system (see, for example, “Neurological Disorders” section below), the immune system (see, for example, “Immune Activity” section below), respiratory system (see, for example, “Respiratory Disorders” section below), cardiovascular system (see, for example, “Cardiovascular Disorders” and/or “Anti-Angiogenesis Acitivity” section below), reproductive system (see, for example, “Reproductive System Disorders” section below), digestive system (see, for example, “Gastrointestinal Disorders” section below), diseases and/or disorders relating to cell proliferation (see, for example, “Hyperproliferative Disorders” section below), and/or diseases or disorders relating to the blood ((see, for example, “Blood-Related Disorders” section below).

In certain embodiments, an SFP-POI of the present invention may be used to diagnose and/or prognose diseases and/or disorders associated with the tissue(s) in which the gene corresponding to the protein of interest portion of the fusion protein of the invention is expressed.

Thus, SFP-POIs of the invention and polynucleotides encoding SFP-POIsof the invention are useful in the diagnosis, detection and/or treatment of diseases and/or disorders associated with activities that include, but are not limited to, prohormone activation, neurotransmitter activity, cellular signaling, cellular proliferation, cellular differentiation, and cell

More generally, SFP-POIs of the invention and polynucleotides encoding SFP-POIs of the invention may be useful for the diagnosis, prognosis, prevention and/or treatment of diseases and/or disorders associated with the following systems.

### Chemotaxis

SFP-POIs of the invention and/or polynucleotides encoding SFP-POIs of the invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

SFPs-POIs of the invention and/or polynucleotides encoding SFP-POIs of the invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that SFP-POIs of the invention and/or polynucleotides encoding SFP-POIs of the invention may inhibit chemotactic activity. These molecules could also be used to treat disorders. Thus, fusion proteins of the invention and/or polynucleotides encoding SFP-POIs of the invention could be used as an inhibitor of chemotaxis.

### Binding Activity

SFP-POIs of the invention may be used to screen for molecules that bind to the protein of interest portion of the SFP-POI or for molecules to which the protein of interest portion of the SFP-POI binds. The binding of the fusion protein and the molecule may activate (agonist), increase, inhibit (antagonist), or decrease activity of the fusion protein or the molecule bound. Examples of such molecules include antibodies, oligonucleotides, proteins (e.g., receptors), or small molecules.

Preferably, the molecule is closely related to the natural ligand of the protein of interest portion of the SFP-POI of the invention, e.g., a fragment of the ligand, or a natural substrate, a ligand, a structural or functional mimetic. (See, Coligan et al., Current Protocols in Immunology 1(2):Chapter 5 (1991)). Similarly, the molecule can be closely related to the natural receptor to which the protein of interest portion of an SFP-POI of the invention binds, or at least, a fragment of the receptor capable of being bound by the protein of interest of an SFP-POI of the invention (e.g., active site). In either case, the molecule can be rationally designed using known techniques.

Preferably, the screening for these molecules involves producing appropriate cells which express the SFP-POIs of the invention. Preferred cells include cells from mammals, yeast, Drosophila, or E. coli. The assay may simply test binding of a candidate compound to an SFP-POI of the invention, wherein binding is detected by a label, or in an assay involving competition with a labeled competitor. Further, the assay may test whether the candidate compound results in a signal generated by binding to the fusion protein.

Alternatively, the assay can be carried out using cell-free preparations, fusion protein/molecule affixed to a solid support, chemical libraries, or natural product mixtures. The assay may also simply comprise the steps of mixing a candidate compound with a solution containing an SFP, measuring SFP-POI/molecule activity or binding, and comparing the fusion protein/molecule activity or binding to a standard.

Preferably, an ELISA assay can measure SFP-POI level or activity in a sample (e.g., biological sample) using a monoclonal or polyclonal antibody. The antibody can measure SFP-POI level or activity by either binding, directly or indirectly, to the SFP-POI or by competing with the SFP-POI for a substrate.

Additionally, the receptor to which a protein of interest portion of an SFP-POI of the invention binds can be identified by numerous methods known to those of skill in the art, for example, ligand panning and FACS sorting (Coligan, et al., Current Protocols in Immun., 1(2), Chapter 5, (1991)). For example, in cases wherein the protein of interest portion of the fusion protein corresponds to FGF, expression cloning may be employed wherein polyadenylated RNA is prepared from a cell responsive to the SFP-POI, for example, NIH3T3 cells which are known to contain multiple receptors for the FGF family proteins, and SC-3 cells, and a cDNA library created from this RNA is divided into pools and used to transfect COS cells or other cells that are not responsive to the SFP. Transfected cells which are grown on glass slides are exposed to the SFP-POI of the present invention, after they have been labeled. The SFP-POIs can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, a labeled SFP-POI can be photoaffinity linked with cell membrane or extract preparations that express the receptor molecule for the protein of interest component of an SFP of the invention, the linked material may be resolved by PAGE analysis and exposed to X-ray film. The labeled complex containing the receptors of the fusion protein can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling and/or codon-shuffling (collectively referred to as “DNA shuffling”) may be employed to modulate the activities of the SFP-POI, and/or therapeutic protein portion or albumin superfamily component of an SFP-POI of the present invention, thereby effectively generating agonists and antagonists of an SFP-POI of the present invention. See generally, U.S. Pat. Nos. 5,605,793, 5,811,238, 5,830,721, 5,834,252, and 5,837,458, and Patten, P. A., et al., Curt. Opinion Biotechnol. 8:724-33 (1997); Harayama, S. Trends Biotechnol. 16(2):76-82 (1998); Hansson, L. O., et al., J. Mol. Biol. 287:265-76 (1999); and Lorenzo, M. M. and Blasco, R. Biotechniques 24(2):308-13 (1998); each of these patents and publications are hereby incorporated by reference). In one embodiment, alteration of polynucleotides encoding SFP-POIs of the invention and thus, the SFP-POI encoded thereby, may be achieved by DNA shuffling. DNA shuffling involves the assembly of two or more DNA segments into a desired molecule by homologous, or site-specific, recombination. In another embodiment, polynucleotides encoding SFP-POIs of the invention and thus, the SFP-POIs encoded thereby, may be altered by being subjected to random mutagenesis by error-prone PCR, random nucleotide insertion or other methods prior to recombination. In another embodiment, one or more components, motifs, sections, parts, domains, fragments, etc., of an SFP-POI of the present invention may be recombined with one or more components, motifs, sections, parts, domains, fragments, etc. of one or more heterologous molecules. In preferred embodiments, the heterologous molecules are family members. In further preferred embodiments, the heterologous molecule is a growth factor such as, for example, platelet-derived growth factor (PDGF), insulin-like growth factor (IGF-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-7, activins A and B, decapentaplegic(dpp), 60A, OP-2, dorsalin, growth differentiation factors (GDFs), nodal, MIS, inhibin-alpha, TGF-beta1, TGF-beta2, TGF-beta3, TGF-beta5, and glial-derived neurotrophic factor (GDNF).

Other preferred fragments are biologically active fragments of the protein of interest portion and/or albumin superfamily component of the SFP-POIs of the present invention. Biologically active fragments are those exhibiting activity similar, but not necessarily identical, to an activity of a protein of interest portion and/or albumin superfamily component of the SFP-POIs of the present invention. The biological activity of the fragments may include an improved desired activity, or a decreased undesirable activity.

Additionally, this invention provides a method of screening compounds to identify those which modulate the action of an SFP-POI of the present invention. An example of such an assay comprises combining a mammalian fibroblast cell, an SFP-POI of the present invention, and the compound to be screened and thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of thymidine in each case. The amount of fibroblast cell proliferation is measured by liquid scintillation chromatography which measures the incorporation of thymidine. Both agonist and antagonist compounds may be identified by this procedure.

In another method, a mammalian cell or membrane preparation expressing a receptor for the protein of interest component of a fusion protein of the invention is incubated with a labeled fusion protein of the present invention in the presence of the compound. The ability of the compound to enhance or block this interaction could then be measured. Alternatively, the response of a known second messenger system following interaction of a compound to be screened and the receptor is measured and the ability of the compound to bind to the receptor and elicit a second messenger response is measured to determine if the compound is a potential fusion protein. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat disease or to bring about a particular result in a patient (e.g., blood vessel growth) by activating or inhibiting the fusion protein/molecule. Moreover, the assays can discover agents which may inhibit or enhance the production of SFPs of the invention from suitably manipulated cells or tissues.

Therefore, the invention includes a method of identifying compounds which bind to an SFP-POI of the invention comprising the steps of: (a) incubating a candidate binding compound with an SFP-POI of the present invention; and (b) determining if binding has occurred. Moreover, the invention includes a method of identifying agonists/antagonists comprising the steps of: (a) incubating a candidate compound with an SFPPOI of the present invention, (b) assaying a biological activity, and (b) determining if a biological activity of the SFP-POI has been altered.

### Targeted Delivery

In another embodiment, the invention provides a method of delivering compositions to targeted cells expressing a receptor for a component of an SFP-POI of the invention. As discussed herein, SFP-POIs of the invention may be associated with invention.

### Binding Peptides and Other Molecules

The invention also encompasses screening methods for identifying polypeptides and nonpolypeptides that bind SFP-POIs of the invention, and the binding molecules identified thereby. These binding molecules are useful, for example, as agonists and antagonists of the SFP-POIs of the invention. Such agonists and antagonists can be used, in accordance with the invention, in the therapeutic embodiments described in detail, below.

This method comprises the steps of: (a) contacting an SFP-POI of the invention with a plurality of molecules; and (b) identifying a molecule that binds the synthetic fusion protein.

The step of contacting the SFP-POI of the invention with the plurality of molecules may be effected in a number of ways. For example, one may contemplate immobilizing the SFP-POI on a solid support and bringing a solution of the plurality of molecules in contact with the immobilized polypeptides. Such a procedure would be akin to an affinity chromatographic process, with the affinity matrix being comprised of the immobilized SFP-POI of the invention. The molecules having a selective affinity for the SFP-POI can then be purified by affinity selection. The nature of the solid support, process for attachment of the SFP-POI to the solid support, solvent, and conditions of the affinity isolation or selection are largely conventional and well known to those of ordinary skill in the art.

Alternatively, one may also separate a plurality of polypeptides into substantially separate fractions comprising a subset of or individual polypeptides. For instance, one can separate the plurality of polypeptides by gel electrophoresis, column chromatography, or like method known to those of ordinary skill for the separation of polypeptides. The individual polypeptides can also be produced by a transformed host cell in such a way as to be expressed on or about its outer surface (e.g., a recombinant phage). Individual isolates can then be “probed” by an SFP-POI of the invention, optionally in the presence of an inducer should one be required for expression, to determine if any selective affinity interaction takes place between the SFP-POI and the individual clone. Prior to contacting the SFP-POI with each fraction comprising individual polypeptides, the polypeptides could first be transferred to a solid support for additional convenience. Such a solid support may simply be a piece of filter membrane, such as one made of nitrocellulose or heterologous polypeptides, heterologous nucleic acids, toxins, or prodrugs via hydrophobic, hydrophilic, ionic and/or covalent interactions. In one embodiment, the invention provides a method for the specific delivery of compositions of the invention to cells by administering fusion proteins of the invention (including antibodies) that are associated with heterologous polypeptides or nucleic acids. In one example, the invention provides a method for delivering a protein of interest into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

In another embodiment, the invention provides a method for the specific destruction of cells (e.g., the destruction of tumor cells) by administering an SFP-POI with toxins or cytotoxic prodrugs.

By “toxin” is meant compounds that bind and activate endogenous cytotoxic effector systems, radioisotopes, holotoxins, modified toxins, catalytic subunits of toxins, or any molecules or enzymes not normally present in or on the surface of a cell that under defined conditions cause the cell's death. Toxins that may be used according to the methods of the invention include, but are not limited to, radioisotopes known in the art, compounds such as, for example, antibodies (or complement fixing containing portions thereof) that bind an inherent or induced endogenous cytotoxic effector system, thymidine kinase, endonuclease, RNAse, alpha toxin, ricin, abrin, Pseudomonas exotoxin A, diphtheria toxin, saporin, momordin, gelonin, pokeweed antiviral protein, alpha-sarcin and cholera toxin. By “cytotoxic prodrug” is meant a non-toxic compound that is converted by an enzyme, normally present in the cell, into a cytotoxic compound. Cytotoxic prodrugs that may be used according to the methods of the invention include, but are not limited to, glutamyl derivatives of benzoic acid mustard alkylating agent, phosphate derivatives of etoposide or mitomycin C, cytosine arabinoside, daunorubisin, and phenoxyacetamide derivatives of doxorubicin.

### Drug Screening

Further contemplated is the use of the SFP-POIs of the present invention, or the polynucleotides encoding these fusion proteins, to screen for molecules which modify the activities of the SFP-POI of the present invention or proteins corresponding to the protein of interest portion of the SFP-POI. Such a method would include contacting the fusion protein with a selected compound(s) suspected of having antagonist or agonist activity, and assaying the activity of the fusion protein following binding.

This invention is particularly useful for screening therapeutic compounds by using the SFP-POI of the present invention, or binding fragments thereof, in any of a variety of drug screening techniques. The SFP-POI employed in such a test may be affixed to a solid support, expressed on a cell surface, free in solution, or located intracellularly. One method of drug screening utilizes eukaryotic or prokaryotic host cells which are stably transformed with recombinant nucleic acids expressing the SFP-POI. Drugs are screened against such transformed cells or supernatants obtained from culturing such cells, in competitive binding assays. One may measure, for example, the formulation of complexes between the agent being tested and an SFP-POI of the present invention.

Thus, the present invention provides methods of screening for drugs or any other agents which affect activities mediated by the SFP-POIs of the present invention. These methods comprise contacting such an agent with an SFP-POI of the present invention or a fragment thereof and assaying for the presence of a complex between the agent and the SFP-POI or a fragment thereof, by methods well known in the art. In such a competitive binding assay, the agents to screen are typically labeled. Following incubation, free agent is separated from that present in bound form, and the amount of free or uncomplexed label is a measure of the ability of a particular agent to bind to the SFP-POI of the present invention.

Another technique for drug screening provides high throughput screening for compounds having suitable binding affinity to an SFP-POI of the present invention, and is described in great detail in European Patent Application 84/03564, published on Sep. 13, 1984, which is incorporated herein by reference herein. Briefly stated, large numbers of different small peptide test compounds are synthesized on a solid substrate, such as plastic pins or some other surface. The peptide test compounds are reacted with an SFP-POI of the present invention and washed. Bound peptides are then detected by methods well known in the art. Purified SFP-POI may be coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies capable of binding an SFP-POI of the present invention specifically compete with a test compound for binding to the SFP-POI or fragments thereof. In this manner, the antibodies are used to detect the presence of any peptide which shares one or more antigenic epitopes with an SFP-POI of the nylon. In this manner, positive clones could be identified from a collection of transformed host cells of an expression library, which harbor a DNA construct encoding a polypeptide having a selective affinity for an SFP-POI of the invention. Furthermore, the amino acid sequence of the polypeptide having a selective affinity for an SFP-POI of the invention can be determined directly by conventional means or the coding sequence of the DNA encoding the polypeptide can frequently be determined more conveniently. The primary sequence can then be deduced from the corresponding DNA sequence. If the amino acid sequence is to be determined from the polypeptide itself, one may use microsequencing techniques. The sequencing technique may include mass spectroscopy.

In certain situations, it may be desirable to wash away any unbound polypeptides from a mixture of an SFP-POI of the invention and the plurality of polypeptides prior to attempting to determine or to detect the presence of a selective affinity interaction. Such a wash step may be particularly desirable when the SFP-POI of the invention or the plurality of polypeptides are bound to a solid support.

The plurality of molecules provided according to this method may be provided by way of diversity libraries, such as random or combinatorial peptide or nonpeptide libraries which can be screened for molecules that specifically bind an SFP-POI of the invention. Many libraries are known in the art that can be used, e.g., chemically synthesized libraries, recombinant (e.g., phage display libraries), and in vitro translation-based libraries. Examples of chemically synthesized libraries are described in Fodor et al., Science 251:767-773 (1991); Houghten et al.; Nature 354:84-86 (1991); Lam et al., Nature 354:82-84 (1991); Medynski, Bio/Technology 12:709-710 (1994); Gallop et al., J. Medicinal Chemistry 37(9):1233-1251 (1994); Ohlmeyer et al., Proc. Natl. Acad. Sci. USA 90:10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614 1618 (1994); Salmon et al., Proc. Natl. Acad. Sci. USA 90:11708-11712 (1993); PC1 Publication No. WO 93/20242; and Brenner and Lerner, Proc. Natl. Acad. Sci. USA 89:5381-5383 (1992).

Examples of phage display libraries are described in Scott et al., Science 249:386-390 (1990); Devlin et al., Science, 249:404-406 (1990); Christian et al., 1992, J. Mol. Biol. 227:711-718 1992); Lenstra, J. Immunol. Meth. 152:149-157 (1992); Kay et al., Gene 128:59-65 (1993); and PCT Publication No. WO 94/18318 dated Aug. 18, 1994.

In vitro translation-based libraries include but are not limited to those described in PCT Publication No. WO 91/05058 dated Apr. 18, 1991; and Mattheakis et al., Proc. Natl. Acad. Sci. USA 91:9022-9026 (1994).

By way of examples of nonpeptide libraries, a benzodiazepine library (see e.g., Bunin et al., Proc. Natl. Acad. Sci. USA 91:4708-4712 (1994)) can be adapted for use. Peptoid libraries (Simon et al., Proc. Natl. Acad. Sci. USA 89:9367-9371 (1992)) can also be used. Another example of a library that can be used, in which the amide functionalities in peptides have been pei methylated to generate a chemically transformed combinatorial library, is described by Ostresh et al. (Proc. Natl. Acad. Sci. USA 91:11138-11142 (1994)).

The variety of non-peptide libraries that are useful in the present invention is great. For example, Ecker and Crooke (Bio/Technology 13:351-360 (1995) list benzodiazepines, hydantoins, piperazinediones, biphenyls, sugar analogs, beta-mercaptoketones, arylacetic acids, acylpiperidines, benzopyrans, cubanes, xanthines, aminimides, and oxazolones as among the chemical species that form the basis of various libraries.

Non-peptide libraries can be classified broadly into two types: decorated monomers and oligomers. Decorated monomer libraries employ a relatively simple scaffold structure upon which a variety functional groups is added. Often the scaffold will be a molecule with a known useful pharmacological activity. For example, the scaffold might be the benzodiazepine structure.

Non-peptide oligomer libraries utilize a large number of monomers that are assembled together in ways that create new shapes that depend on the order of the monomers. Among the monomer units that have been used are carbamates, pyrrolinones, and morpholinos. Peptoids, peptide-like oligomers in which the side chain is attached to the alpha amino group rather than the alpha carbon, form the basis of another version of non-peptide oligomer libraries. The first non-peptide oligomer libraries utilized a single type of monomer and thus contained a repeating backbone. Recent libraries have utilized more than one monomer, giving the libraries added flexibility.

Screening the libraries can be accomplished by any of a variety of commonly known methods. See, e.g., the following references, which disclose screening of peptide libraries: Parmley et al., Adv. Exp. Med. Biol. 251:215-218 (1989); Scott et al., Science 249:386-390 (1990); Fowlkes et al., BioTechniques 13:422-427 (1992); Oldenburg et al., Proc. Natl. Acad. Sci. USA 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. USA 89:6988-6992 (1992); Ellington et al., Nature 355:850-852 (1992); U.S. Pat. No. 5,096,815, U.S. Pat. No. 5,223,409, and U.S. Pat. No. 5,198,346, all to Ladner et al.; Rebar et al., Science 263:671-673 (1993); and PCT Publication No. WO 94/18318.

In a specific embodiment, screening to identify a molecule that binds an SFP-POI of the invention can be carried out by contacting the library members with an SFP-POI of the invention immobilized on a solid phase and harvesting those library members that bind to the SFP-POI. Examples of such screening methods, termed “panning” techniques are described by way of example in Palmley et al., Gene 73:305-318 (1988); Fowlkes et al., BioTechniques 13:422-427 (1992); PCT Publication No. WO 94/18318; and in references cited herein.

In another embodiment, the two-hybrid system for selecting interacting proteins in yeast (Fields et al., Nature 340:245-246 (1989); Chien et al., Proc. Natl. Acad. Sci. USA 88:9578-9582 (1991) can be used to identify molecules that specifically bind to polypeptides of the invention.

Where the binding molecule is a polypeptide, the polypeptide can be conveniently selected from any peptide library, including random peptide libraries, combinatorial peptide libraries, or biased peptide libraries. The term “biased” is used herein to mean that the method of generating the library is manipulated so as to restrict one or more parameters that govern the diversity of the resulting collection of molecules, in this case peptides.

Thus, a truly random peptide library would generate a collection of peptides in which the probability of finding a particular amino acid at a given position of the peptide is the same for all 20 amino acids. A bias can be introduced into the library, however, by specifying, for example, that a lysine occur every fifth amino acid or that positions 4, 8, and 9 of a decapeptide library be fixed to include only arginine. Clearly, many types of biases can be contemplated, and the present invention is not restricted to any particular bias. Furthermore, the present invention contemplates specific types of peptide libraries, such as phage displayed peptide libraries and those that utilize a DNA construct comprising a lambda phage vector with a DNA insert.

As mentioned above, in the case of a binding molecule that is a polypeptide, the polypeptide may have about 6 to less than about 60 amino acid residues, preferably about 6 to about, 10 amino acid residues, and most preferably, about 6 to about 22 amino acids. In another embodiment, a binding polypeptide has in the range of 15-100 amino acids, or 20-50 amino acids.

The selected binding polypeptide can be obtained by chemical synthesis or recombinant expression.

### Other Activities

An SFP-POI of the invention and/or polynucleotide encoding an SFP-POI of the invention, may be employed in treatment for stimulating re-vascularization of ischemic tissues due to various disease conditions such as thrombosis, arteriosclerosis, and other cardiovascular conditions. The SFP-POIs of the invention and/or polynucleotides encoding SFP-POIs of the invention may also be employed to stimulate angiogenesis and limb regeneration, as discussed above.

An SFP-POI of the invention and/or polynucleotide encoding an SFP-POI of the invention may also be employed for treating wounds due to injuries, burns, post-operative tissue repair, and ulcers since they are mitogenic to various cells of different origins, such as fibroblast cells and skeletal muscle cells, and therefore, facilitate the repair or replacement of damaged or diseased tissue.

An SFP-POI of the invention and/or polynucleotide encoding an SFP-POI protein of the invention may also be employed stimulate neuronal growth and to treat and prevent neuronal damage which occurs in certain neuronal disorders or neuro-degenerative conditions such as Alzheimer's disease, Parkinson's disease, and AIDS-related complex. An SFP-POI of the invention and/or polynucleotide encoding an SFP-POI of the invention may have the ability to stimulate chondrocyte growth, therefore, they may be employed to enhance bone and periodontal regeneration and aid in tissue transplants or bone grafts.

An SFP-POI of the invention and/or polynucleotide encoding an SFP-POI of the invention may be also be employed to prevent skin aging due to sunburn by stimulating keratinocyte growth. An SFP-POI of the invention and/or polynucleotide encoding an SFP-POI of the invention may also be employed for preventing hair loss, since FGF family members activate hair-forming cells and promotes melanocyte growth. Along the same lines, an SFP-POI of the invention and/or polynucleotide encoding an SFP-POI of the invention may be employed to stimulate growth and differentiation of hematopoietic cells and bone marrow cells when used in combination with other cytokines.

An SFP-POI of the invention and/or polynucleotide encoding an SFP-POI of the invention may also be employed to maintain organs before transplantation or for supporting cell culture of primary tissues. An SFP-POI of the invention and/or polynucleotide encoding an SFP-POI of the invention may also be employed for inducing tissue of mesodermal origin to differentiate in early embryos.

An SFP-POI of the invention and/or polynucleotide encoding an SFP-POI of the invention may also increase or decrease the differentiation or proliferation of embryonic stem cells, besides, as discussed above, hematopoietic lineage.

An SFP-POI of the invention and/or polynucleotide encoding an SFP-POI of the invention may also be used to modulate mammalian characteristics, such as body height, weight, hair color, eye color, skin, percentage of adipose tissue, pigmentation, size, and shape (e.g., cosmetic surgery). Similarly, an SFP-POI of the invention and/or polynucleotide encoding an SFP-POI of the invention may be used to modulate mammalian metabolism affecting catabolism, anabolism, processing, utilization, and storage of energy.

An SFP-POI of the invention and/or polynucleotide encoding an SFP-POI of the invention may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

An SFP-POI of the invention and/or polynucleotide encoding an SFP-POI of the invention may also be used as a food additive or preservative, such as to increase or decrease storage capabilities, fat content, lipid, protein, carbohydrate, vitamins, minerals, cofactors or other nutritional components.

The above-recited applications have uses in a wide variety of hosts. Such hosts include, but are not limited to, human, murine, rabbit, goat, guinea pig, camel, horse, mouse, rat, hamster, pig, micro-pig, chicken, goat, cow, sheep, dog, cat, non-human primate, and human. In specific embodiments, the host is a mouse, rabbit, goat, guinea pig, chicken, rat, hamster, pig, sheep, dog or cat. In preferred embodiments, the host is a mammal. In most preferred embodiments, the host is a human.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out preferred embodiments of the present invention, and are not to be construed as limiting in any way the remainder of the disclosure.

### EXAMPLES

### Example 1

### Insertion of the Factor IX Gene into the Human Albumin Locus

Factor IX (FIX) is one member of the blood coagulation cascade and is deficient in patients with hemophilia B (11). It is produced in the liver but is synthesized at a relatively low level. To insert the FIX cDNA into the albumin locus, a targeting plasmid containing the FIX cDNA and two plasmids coding for a dimeric TALEN that cut the human albumin gene at nucleotide 93 were cotransfected into C3A cells using electroporation. The targeting plasmid contains a selectable marker, in this case neomycin resistance. The resulting G418 resistant clones expressed high levels of human factor IX and secreted it into the cell culture medium.

The TALENs were designed to bind to the sequence TCTTTTTCTCTTTAGCTCG (SEQ ID NO: 8) on the 5′ side of the cut site and to the sequence TACGTGCATCTCGACGAAA (SEQ ID NO: 9) on the 3′ side. The TALENs were obtained from Life Technologies, Inc.

The targeting plasmid was constructed using seamless gene cloning. In brief, overlapping oligonucleotides are synthesized joining the desired segments in the construct. These are then assembled from PCR products, ligated and transfected. The required enzymes were obtained from Life Technologies. Four PCR products were amplified. The plasmid pUC19 was used as the vehicle. The factor IX insert was amplified from pEX-T0161-M51, obtained from Genecopoeia. The 5′ and 3′ flanking DNAs were obtained from C3A genomic DNA. These were ligated in sequence and transfected into E. coli. The appropriate plasmids were identified by restriction digestion. The data demonstrating construction of the FIX targeting plasmid are shown in FIG. 4.

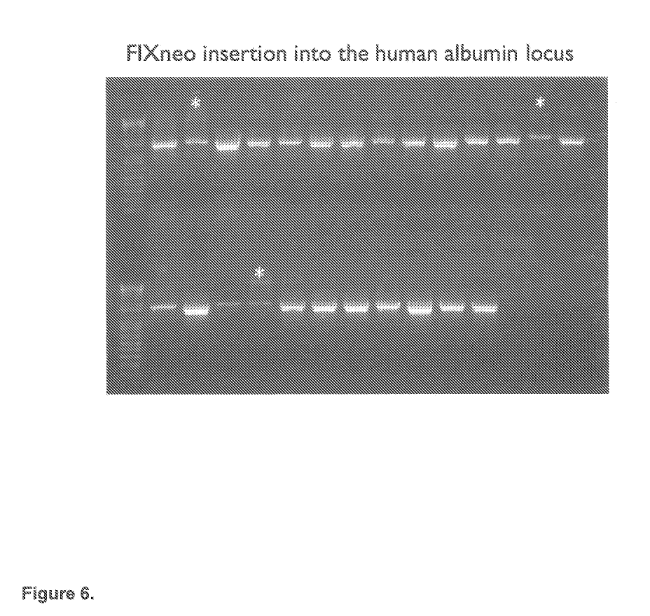

After approximately three weeks in 500 μg/ml G418, a panel of resistant clones were selected, expanded and analyzed for insertion of the FIX gene into the albumin locus. Using a primer located in the 5′ flanking DNA of the human albumin gene, and contained within the inserted construct, and another primer located in the first intervening sequence of the human albumin gene but not within the construct, three clones were identified as having the correct insertion by PCR. These are designated clone 2, 13, and 18 respectively. Results are shown in FIG. 6.

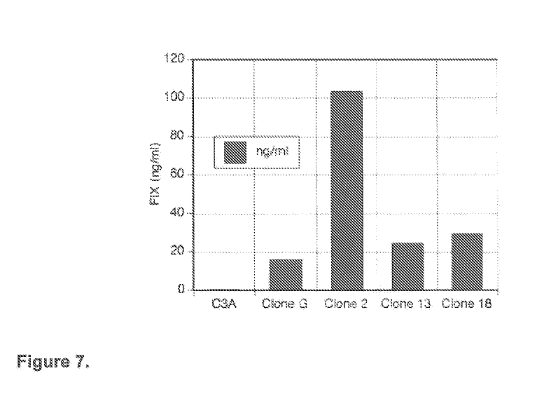

The clones were analyzed for secretion of FIX into the culture medium by enzyme linked immunoassay (ELISA). Medium without bovine calf serum was placed on the cells for 24 hrs, collected and analyzed using a commercial kit obtained from Abcam, Inc. Results are shown in FIG. 7.

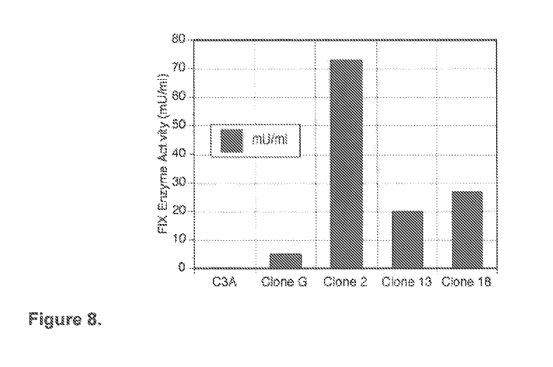

Next, the FIX was assayed for enzyme activity. FIX is a vitamin K dependent enzyme, a component not normally found in cell culture medium. Medium containing 5 μg/ml vitamin K was added to the cultures for 24 hrs, then the supernatant fluid was assayed for FIX activity using a commercial kit obtained from Aniara, Inc. Results are shown in FIG. 8.

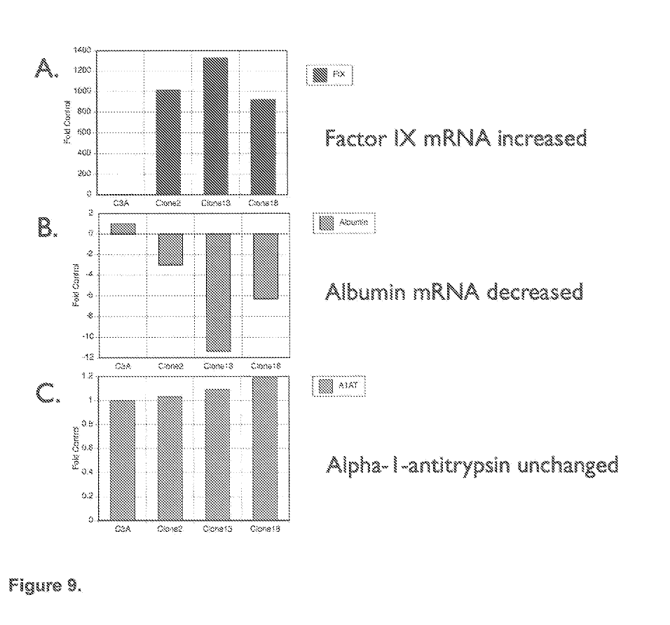

Next, mRNA levels were measured using a TaqMan assay obtained from Life Technologies, Inc. RNA was isolated from C3A cells, clones 2, 13 and 18. The mRNA for FIX, albumin, alpha-1-antitrypsin (A1AT) and glyceraldeyhde-3-phosphate dehydrogenase (GAPDH) were measured. Results are shown in FIG. 9.

These results demonstrate that insertion into the human albumin locus gives consistent high level expression of the inserted cDNA. Albumin, A1AT and GAPDH are three of the most abundant mRNAs in C3A cells. FIX expressed from the albumin locus is comparable to these genes.

### Example 2

### Insertion of the Stabile9 (Factor IX-SFP Fusion Gene) into the Human Albumin Locus

Stabile9 is a fusion gene between factor IX and SFP. The gene was synthesized by Life Technologies, Inc. The amino acid sequence of Stabile9 is shown in SEQ ID NO: 11. It consists of the signal sequence from human albumin, the sequence of FIX without its own signal sequence, a linker region and the sequence of SFP. A targeting plasmid similar to that described for FIX was constructed except that Stabile9 was substituted for FIX and transfected with the same TALEN encoding plasmids. The resulting G-418 resistant clones synthesized Stabile9 from the human albumin locus.

### Example 3

### Insertion of StabileBChE (Butyrylcholinesterase-SFP Fusion Gene) into the Human Albumin Locus

StabileBChE is a fusion gene between butyrylcholinesterase (BChE) and SFP. BChE can be used to protect against organophosphorus nerve agents (Lenz, D E, et al. (2007) Stoichiometric and catalytic scavengers as protection against nerve agent toxicity: a mini review. Toxicology 233, 31-39). The amino acid sequence of StabileBChE is shown in SEQ ID NO: 12. It consists of the signal sequence from human albumin, the sequence of BChE without its own signal sequence, a linker region and the sequence of SFP. The gene was synthesized by Life Technologies, Inc. A targeting plasmid was constructed and transfected similarly to that described for FIX except that the cDNA for StabileBChE was substituted. The resulting clones synthesized StabileBChE from the human albumin locus.

### Example 4

### Insertion of Stabile8 (Factor VIII-SFP Fusion Gene) into the Albumin Locus and Insertion of Von Willebrand Factor into the Transferrin Locus

Factor VIII is another member of the coagulation cascade and is deficient in patients with hemophilia A, the more common form of hemophilia (Bergman, G E (2011) Progress in treatment of bleeding disorders. Thromb. Res. 127, Supp11: S3-5). Synthesized on its own, it is highly unstable and requires a second factor, von Willebrand Factor (vWF). Stabile8, a long half-life version of Factor VIII, is further stabilized by vWF. By inserting one gene into the albumin locus and another into a second highly synthesized gene, in this case the transferrin gene, production of both proteins can be matched.

### Example 5

### Maximizing Expression Via Codon Optimization and a Minigene Construct

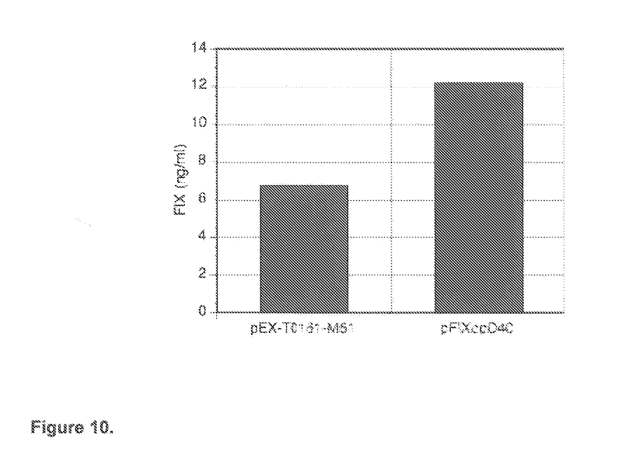

Having demonstrated that insertion into the albumin locus yields high level transcription of the insert, a further maximization of expression was desired. A codon optimized FIX cDNA (provided herein) was synthesized and inserted into pcDNA-DEST40, creating a plasmid (pFIXopD40) that drives FIX synthesis from a CMV promoter. Transient expression experiments compared expression from pFIXopD40 to pEX-T0161-M51, which utilizes a CMV promoter to drive the natural cDNA. Results are shown in FIG. 10.

Other modifications that increase expression include the addition of an intervening sequence and inclusion of a stabilizing 3′ untranslated region within the RNA. A factor IX mini gene (pFIXmini) was constructed that contained all three of these modifications: a codon optimized cDNA, an intervening sequence and a 3′ untranslated region (See FIG. 11). The construct contains sequences from exon 14, intron 14 and exon15 of the human albumin gene. The construct also contains an internal ribosome entry site driving the neo gene for G418 resistance.

The mini gene construct was inserted into the human albumin locus. pFIXmini contains about 1000 bp of DNA from the 5′ flanking DNA of the human albumin gene and about 1000 bp of DNA from the 3′ end of the gene. These sequences are sufficient for homologous recombination with the addition of site specific nucleases. The Cas-CRISPR system is an RNA guided site specific nuclease that can be used for homologous recombination. Tandem sets of guide sequences were identified from the 5′ and 3′ ends of the albumin gene and incorporated into Cas-CRISPR plasmids from System Biosciences. Homologous recombination of pFIXmini into the albumin locus results in deletion of the bulk of the albumin gene. Using nucleoporation, 5 μg of pFIXmini and 1 μg each of the four CasCRSPR plasmids were transfected into 1 million C3A cells. Resistant clones were selected in 500 μg/ml G418.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

1. Wurm, F M (2004) Production of recombinant proteins in cultivated mammalian cells. Nat. Biotechnol. 22, 1393-1398.
2. Ward, E S, Ober, R J (2009) Multitasking by exploitation of intracellular transport functions: the many faces of FcRN. Adv. Immunol 103, 77-115.
3. Chaudhury C, et al. (2003) The major histocompatibility complex-related Fc receptor for IgG (FcRN) binds albumin and prolongs its lifespan. J. Exp. Med. 197, 315-322.
4. Andersen, J T, et al. (2010) Cross-species binding analyses fo mouse and human neonatal Fc receptor show dramatic differences in immunoglobulin G and albumin binding. J. Biol. Chem. 285, 4826-4836.
5. Peters, T. (1996) ALL ABOUT ALBUMIN, Academic Press, San Diego, Calif., 423 pp.
6. Peavy, D E, et al. (1978) Cporrelation of albumin production rates and albumin mRNA levels in livers of normal, diabetic, and insulin-treated diabetic rats. Proc. Natl. Acad. Sci. 75, 5879-5883.
7. Bowman, B H (1993) HEPATIC PLASMA PROTEINS, Academic Press, San Diego, Calif.
8. Kelly, J H (1994) U.S. Pat. No. 5,290,684. Permanent human hepatocyte cell line and its use in a liver assist device (LAD).
9. Miller, J C, et al. (2010) A TALE nuclease architecture for efficient genome engineering. Nat. Biotechnol. 29, 143-148.
10. Durai, S, et al. (2005) Zinc finger nucleases:custom-designed molecular scissors for genome engineering of plant and mammalian cells. Nuc. Acids. Res. 33, 5978-5990.
11. Kawaguchi, T, et al. (2011) Branched-chain amino acids as pharmacological nutrients in chronic liver disease. Hepatology 54, 1063-1070.
12. Moriwaki, H, et al. (2004) Branched chain amino acids as a protein- and energy-source in liver cirrhosis. Biochem. Biophys. Res. 313, 405-409.
13. Ellisen, L W (2005) Growth control under stress: mTOR regulation through the REDD1-TSC pathway. Cell Cycle 4, 1500-1502.
14. Bergman, G E (2011) Progress in treatment of bleeding disorders. Thromb. Res. 127, Supp11: S3-5.
15. Lenz, D E, et al. (2007) Stoichiometric and catalytic scavengers as protection against nerve agent toxicity: a mini review. Toxicology 233, 31-39.

```
SEQUENCES
Human Albumin mRNA (SEQ ID NO: 1)
NCBI Reference Sequence NM_000477.5
GenBank V00494
      1 agtatattag tgctaatttc cctccgtttg tcctagcttt tctcttctgt caaccccaca
    61 cgcctttggc acaatgaagt gggtaacctt tatttccctt ctttttctct ttagctcggc
  121 ttattccagg ggtgtgtttc gtcgagatgc acacaagagt gaggttgctc atcggtttaa
  181 agatttggga gaagaaaatt tcaaagcctt ggtgttgatt gcctttgctc agtatcttca
  241 gcagtgtcca tttgaagatc atgtaaaatt agtgaatgaa gtaactgaat ttgcaaaaac
  301 atgtgttgct gatgagtcag ctgaaaattg tgacaaatca cttcataccc tttttggaga
  361 caaattatgc acagttgcaa ctcttcgtga aacctatggt gaaatggctg actgctgtgc
  421 aaaacaagaa cctgagagaa atgaatgctt cttgcaacac aaagatgaca acccaaacct
  481 cccccgattg gtgagaccag aggttgatgt gatgtgcact gcttttcatg acaatgaaga
  541 gacatttttg aaaaaatact tatatgaaat tgccagaaga catccttact tttatgcccc
  601 ggaactcctt ttctttgcta aaaggtataa agctgctttt acagaatgtt gccaagctgc
  661 tgataaagct gcctgcctgt tgccaaagct cgatgaactt cgggatgaag ggaaggcttc
  721 gtctgccaaa cagagactca agtgtgccag tctccaaaaa tttggagaaa gagctttcaa
  781 agcatgggca gtagctcgcc tgagccagag atttcccaaa gctgagtttg cagaagtttc
  841 caagttagtg acagatctta ccaaagtcca cacggaatgc tgccatggag atctgcttga
  901 atgtgctgat gacagggcgg accttgccaa gtatatctgt gaaaatcaag attcgatctc
  961 cagtaaactg aaggaatgct gtgaaaaacc tctgttggaa aaatcccact gcattgccga
1021 agtggaaaat gatgagatgc ctgctgactt gccttcatta gctgctgatt ttgttgaaag
1081 taaggatgtt tgcaaaaact atgctgaggc aaaggatgtc ttcctgggca tgtttttgta
1141 tgaatatgca agaaggcatc ctgattactc tgtcgtgctg ctgctgagac ttgccaagac
1201 atatgaaacc actctagaga agtgctgtgc cgctgcagat cctcatgaat gctatgccaa
1261 agtgttcgat gaatttaaac ctcttgtgga agagcctcag aatttaatca aacaaaattg
1321 tgagcttttt gagcagcttg gagagtacaa attccagaat gcgctattag ttcgttacac
1381 caagaaagta ccccaagtgt caactccaac tcttgtagag gtctcaagaa acctaggaaa
1441 agtgggcagc aaatgttgta aacatcctga agcaaaaaga atgccctgtg cagaagacta
1501 tctatccgtg gtcctgaacc agttatgtgt gttgcatgag aaaacgccag taagtgacag
1561 agtcaccaaa tgctgcacag aatccttggt gaacaggcga ccatgctttt cagctctgga
1621 agtcgatgaa acatacgttc ccaaagagtt taatgctgaa acattcacct tccatgcaga
1681 tatatgcaca ctttctgaga aggagagaca aatcaagaaa caaactgcac ttgttgagct
1741 cgtgaaacac aagcccaagg caacaaaaga gcaactgaaa gctgttatgg atgatttcgc
1801 agcttttgta gagaagtgct gcaaggctga cgataaggag acctgctttg ccgaggaggg
1861 taaaaaactt gttgctgcaa gtcaagctgc cttaggctta taacatcaca tttaaaagca
1921 tctcagccta ccatgagaat aagagaaaga aaatgaagat caaaagctta ttcatctgtt
1981 tttctttttc gttggtgtaa agccaacacc ctgtctaaaa aacataaatt tctttaatca
2041 ttttgcctct tttctctgtg cttcaattaa taaaaaatgg aaagaatcta atagagtggt
2101 acagcactgt tatttttcaa agatgtgttg ctatcctgaa aattctgtag gttctgtgga
2161 agttccagtg ttctctctta ttccacttcg gtagaggatt tctagtttct tgtgggctaa
2221 ttaaataaat cattaatact cttctaaaaa aaaaaaaaaa aaaa
Human Alphafetoprotein mRNA (SEQ ID NO: 2)
NCBI Reference Sequence NM_001134.1
GenBank V01514.1
      1 tccatattgt gcttccacca ctgccaataa caaaataact agcaaccatg aagtgggtgg
    61 aatcaatttt tttaattttc ctactaaatt ttactgaatc cagaacactg catagaaatg
  121 aatatggaat agcttccata ttggattctt accaatgtac tgcagagata agtttagctg
  181 acctggctac catatttttt gcccagtttg ttcaagaagc cacttacaag gaagtaagca
  241 aaatggtgaa agatgcattg actgcaattg agaaacccac tggagatgaa cagtcttcag
  301 ggtgtttaga aaaccagcta cctgcctttc tggaagaact ttgccatgag aaagaaattt
  361 tggagaagta cggacattca gactgctgca gccaaagtga agagggaaga cataactgtt
  421 ttcttgcaca caaaaagccc actccagcat cgatcccact tttccaagtt ccagaacctg
  481 tcacaagctg tgaagcatat gaagaagaca gggagacatt catgaacaaa ttcatttatg
  541 agatagcaag aaggcatccc ttcctgtatg cacctacaat tcttctttgg gctgctcgct
  601 atgacaaaat aattccatct tgctgcaaag ctgaaaatgc agttgaatgc ttccaaacaa
  661 aggcagcaac agttacaaaa gaattaagag aaagcagctt gttaaatcaa catgcatgtg
  721 cagtaatgaa aaattttggg acccgaactt tccaagccat aactgttact aaactgagtc
  781 agaagtttac caaagttaat tttactgaaa tccagaaact agtcctggat gtggcccatg
  841 tacatgagca ctgttgcaga ggagatgtgc tggattgtct gcaggatggg gaaaaaatca
  901 tgtcctacat atgttctcaa caagacactc tgtcaaacaa aataacagaa tgctgcaaac
  961 tgaccacgct ggaacgtggt caatgtataa ttcatgcaga aaatgatgaa aaacctgaag
1021 gtctatctcc aaatctaaac aggtttttag gagatagaga ttttaaccaa ttttcttcag
1081 gggaaaaaaa tatcttcttg gcaagttttg ttcatgaata ttcaagaaga catcctcagc
1141 ttgctgtctc agtaattcta agagttgcta aaggatacca ggagttattg gagaagtgtt
1201 tccagactga aaaccctctt gaatgccaag ataaaggaga agaagaatta cagaaataca
1261 tccaggagag ccaagcattg gcaaagcgaa gctgcggcct cttccagaaa ctaggagaat
1321 attacttaca aaatgcgttt ctcgttgctt acacaaagaa agccccccag ctgacctcgt
1381 cggagctgat ggccatcacc agaaaaatgg cagccacagc agccacttgt tgccaactca
1441 gtgaggacaa actattggcc tgtggcgagg gagcggctga cattattatc ggacacttat
1501 gtatcagaca tgaaatgact ccagtaaacc ctggtgttgg ccagtgctgc acttcttcat
1561 atgccaacag gaggccatgc ttcagcagct tggtggtgga tgaaacatat gtccctcctg
1621 cattctctga tgacaagttc attttccata aggatctgtg ccaagctcag ggtgtagcgc
1681 tgcaaacgat gaagcaagag tttctcatta accttgtgaa gcaaaagcca caaataacag
1741 aggaacaact tgaggctgtc attgcagatt tctcaggcct gttggagaaa tgctgccaag
1801 gccaggaaca ggaagtctgc tttgctgaag agggacaaaa actgatttca aaaactcgtg
1861 ctgctttggg agtttaaatt acttcagggg aagagaagac aaaacgagtc tttcattcgg
1921 tgtgaacttt tctctttaat tttaactgat ttaacacttt ttgtgaatta atgaaatgat
1981 aaagactttt atgtgagatt tccttatcac agaaataaaa tatctccaaa tg
Human Vitamin D Binding Protein (SEQ ID NO: 3)
NCBI Reference Sequence NM_000583.3
GenBank L10641
      1 atattaagta aactttagtg aggaacagca gtggaaaata atctatatac cttggctctt
    61 ttgcagtttg acaaagttaa tgattaaaat ctcctagatt ttccactaca gtatccccag
  121 ggtgtctatt taccttgatt gatattattt tatctctttt gggccaaaga taacagcccc
  181 ttgcttctgt gtttaataat aattctgtgt tgcttctgag attaataatt gattaattca
  241 tagtcaggaa tctttgtaaa aaggaaacca attacttttg gctaccactt ttacatggtc
  301 acctacagga gagaggaggt gctgcaagac tctctggtag aaaaatgaag agggtcctgg
  361 tactactgct tgctgtggca tttggacatg ctttagagag aggccgggat tatgaaaaga
  421 ataaagtctg caaggaattc tcccatctgg gaaaggagga cttcacatct ctgtcactag
  481 tcctgtacag tagaaaattt cccagtggca cgtttgaaca ggtcagccaa cttgtgaagg
  541 aagttgtctc cttgaccgaa gcctgctgtg cggaaggggc tgaccctgac tgctatgaca
  601 ccaggacctc agcactgtct gccaagtcct gtgaaagtaa ttctccattc cccgttcacc
  661 caggcactgc tgagtgctgc accaaagagg gcctggaacg aaagctctgc atggctgctc
  721 tgaaacacca gccacaggaa ttccctacct acgtggaacc cacaaatgat gaaatctgtg
  781 aggcgttcag gaaagatcca aaggaatatg ctaatcaatt tatgtgggaa tattccacta
  841 attacggaca agctcctctg tcacttttag tcagttacac caagagttat ctttctatgg
  901 tagggtcctg ctgtacctct gcaagcccaa ctgtatgctt tttgaaagag agactccagc
  961 ttaaacattt atcacttctc accactctgt caaatagagt ctgctcacaa tatgctgctt
1021 atggggagaa gaaatcaagg ctcagcaatc tcataaagtt agcccaaaaa gtgcctactg
1081 ctgatctgga ggatgttttg ccactagctg aagatattac taacatcctc tccaaatgct
1141 gtgagtctgc ctctgaagat tgcatggcca aagagctgcc tgaacacaca gtaaaactct
1201 gtgacaattt atccacaaag aattctaagt ttgaagactg ttgtcaagaa aaaacagcca
1261 tggacgtttt tgtgtgcact tacttcatgc cagctgccca actccccgag cttccagatg
1321 tagagttgcc cacaaacaaa gatgtgtgtg atccaggaaa caccaaagtc atggataagt
1381 atacatttga actaagcaga aggactcatc ttccggaagt attcctcagt aaggtacttg
1441 agccaaccct aaaaagcctt ggtgaatgct gtgatgttga agactcaact acctgtttta
1501 atgctaaggg ccctctacta aagaaggaac tatcttcttt cattgacaag ggacaagaac
1561 tatgtgcaga ttattcagaa aatacattta ctgagtacaa gaaaaaactg gcagagcgac
1621 taaaagcaaa attgcctgat gccacaccca cggaactggc aaagctggtt aacaagcact
1681 cagactttgc ctccaactgc tgttccataa actcacctcc tctttactgt gattcagaga
1741 ttgatgctga attgaagaat atcctgtagt cctgaagcat gtttattaac tttgaccaga
1801 gttggagcca cccaggggaa tgatctctga tgacctaacc taagcaaaac cactgagctt
1861 ctgggaagac aactaggata ctttctactt tttctagcta caatatcttc atacaatgac
1921 aagtatgatg atttgctatc aaaataaatt gaaatataat gcaaaccata aaaaaaaaaa
1981 aaaaaaaaaa aaaaaaaaaa aaaaaaaaaa aaaaaaaaaa aaaa
Human Afamin (SEQ ID NO: 4)
NCBI Refernce Sequence NM_001133
GenBank L32140
      1 actttctttt gtaaatgtgg tttctacaaa gatgaaacta ctaaaactta caggttttat
    61 ttttttcttg ttttttttga ctgaatccct aaccctgccc acacaacctc gggatataga
  121 gaacttcaat agtactcaaa aatttataga agataatatt gaatacatca ccatcattgc
  181 atttgctcag tatgttcagg aagcaacctt tgaagaaatg gaaaagctgg tgaaagacat
  241 ggtagaatac aaagacagat gtatggctga caagacgctc ccagagtgtt caaaattacc
  301 taataatgtt ttacaggaaa aaatatgtgc tatggagggg ctgccacaaa agcataattt
  361 ctcacactgc tgcagtaagg ttgatgctca aagaagactc tgtttcttct ataacaagaa
  421 atctgatgtg ggatttctgc ctcctttccc taccctggat cccgaagaga aatgccaggc
  481 ttatgaaagt aacagagaat cccttttaaa tcacttttta tatgaagttg ccagaaggaa
  541 cccatttgtc ttcgccccta cacttctaac tgttgctgtt cattttgagg aggtggccaa
  601 atcatgttgt gaagaacaaa acaaagtcaa ctgccttcaa acaagggcaa tacctgtcac
  661 acaatattta aaagcatttt cttcttatca aaaacatgtc tgtggggcac ttttgaaatt
  721 tggaaccaaa gttgtacact ttatatatat tgcgatactc agtcaaaaat tccccaagat
  781 tgaatttaag gagcttattt ctcttgtaga agatgtttct tccaactatg atggatgctg
  841 tgaaggggat gttgtgcagt gcatccgtga cacgagcaag gttatgaacc atatttgttc
  901 aaaacaagat tctatctcca gcaaaatcaa agagtgctgt gaaaagaaaa taccagagcg
  961 cggccagtgc ataattaact caaacaaaga tgatagacca aaggatttat ctctaagaga
1021 aggaaaattt actgacagtg aaaatgtgtg tcaagaacga gatgctgacc cagacacctt
1081 ctttgcgaag tttacttttg aatactcaag gagacatcca gacctgtcta taccagagct
1141 tttaagaatt gttcaaatat acaaagatct cctgagaaat tgctgcaaca cagaaaaccc
1201 tccaggttgt taccgttacg cggaagacaa attcaatgag acaactgaga aaagcctcaa
1261 gatggtacaa caagaatgta aacatttcca gaatttgggg aaggatggtt tgaaatacca
1321 ttacctcatc aggctcacga agatagctcc ccaactctcc actgaagaac tggtgtctct
1381 tggcgagaaa atggtgacag ctttcactac ttgctgtacg ctaagtgaag agtttgcctg
1441 tgttgataat ttggcagatt tagtttttgg agagttatgt ggagtaaatg aaaatcgaac
1501 tatcaaccct gctgtggacc actgctgtaa aacaaacttt gccttcagaa ggccctgctt
1561 tgagagtttg aaagctgata aaacatatgt gcctccacct ttctctcaag atttatttac
1621 ctttcacgca gacatgtgtc aatctcagaa tgaggagctt cagaggaaga cagacaggtt
1681 tcttgtcaac ttagtgaagc tgaagcatga actcacagat gaagagctgc agtctttgtt
1741 tacaaatttc gcaaatgtag tggataagtg ctgcaaagca gagagtcctg aagtctgctt
1801 taatgaagag agtccaaaaa ttggcaactg aagccagctg ctggagatat gtaaagaaaa
1861 aagcaccaaa gggaaggctt cctatctgtg tggtgatgaa tcgcatttcc tgagaacaaa
1921 ataaaaggat ttttctgtaa ctgtcacctg aaataataca ttgcagcaag caataaacac
1981 aacattttgt aaagtta
SFP2
(SEQ ID NO: 5)
M K R V L V L L L A V A F G H A L E R G R D Y E K N K V C K E F S H L G K E D F T
S L S L V L Y S R K F P S G T F E Q V S Q L V K E V V S L T E A C V A E G A D P D
C Y D T R T S A L S A K S C E S N S P F P V H P G T A E C C T K E G L E R K L C M
A A L K H Q P Q E F P T Y V E P T N D E I C E A F R K D P K E Y A N Q F M W E Y S
T N Y G Q A P L S L L V S Y T K S Y L S M V G S C C T S A S P T V C F L K E R L Q
L K H L S L L T T L S N R V C S Q Y A A Y G E K K S R L S N L I K L A Q K V P T A
D L E D V L P L A E D I T N I L S K C C E S A S E D C M A K E L P E H T V K L C D
N Q D T K N S K F E D C C Q E K T A M D V F V C T Y F M P A A Q L P E L P D V E L
P T N K D V C D P G N T K V M D K Y T F E L S R R T H L P E V F L S K V L E P T L
K S L G E C C D V E D S T T C F N A K G P L L K K E L S S F I D K G Q E L C A D Y
S E N T F Y Y L Q N A F L V A Y T K K A P Q L T S S E L M A I T R K M A A T A A T
C C Q L S E D K L L A C G E G A A D I I I G H L C I L H E M T P V S D R V T Q C C
T S S Y A N R R P C F S S L E V D E T Y V P K E F S D D K F T F H S D L C Q A Q G
V A L Q T M K Q E F L I N L V K H K P K I T E E Q L E A V I A D F S G L L E K C C
Q G Q E Q E V C F A E E G W K L I S K T R A A L G V
AFP
(SEQ ID NO: 6)
mkwvesiflifllnftesrtlhrneygiasildsyqctaeisladlatiffaqfvqeaty
kevskmvkdaltaiekptgdeqssgclenqlpafleelchekeilekyghsdccsqseeg
rhncflahkkptpasiplfqvpepvtsceayeedretfmnkfiyeiarrhpflyaptill
waarydkiipscckaenavecfqtkaatvtkelressllnqhacavmknfgtrtfqaitv
tklsqkftkvnfteiqklvldvahvhehccrgdvldclqdgekimsyicsqqdtlsnkit
eccklttlergqciihaendekpeglspnlnrflgdrdfnqfssgekniflasfvheysr
rhpqlaysvilrvakgyqellekcfqtenplecqdkgeeelqkyiqesqalakrscglfq
klgeyylqnaflvaytkkapqltsselmaitrkmaataatccqlsedkllacgegaadii
ighlcirhemtpvnpgvgqcctssyanrrpcfsslvvdetyvppafsddkfifhkdlcqa
qgvalqtmkqeflinlvkqkpqiteeqleaviadfsgllekccqgqeqevcfaeegqklisktraalgv
DBP
(SEQ ID NO: 7)
MKRVLVLLLAVAFGHALERGRDYEKNKVCKEFSHLGKEDFTSLSLVLYSRKFPSGTFEQV
SQLVKEVVSLTEACCAEGADPDCYDTRTSALSAKSCESNSPFPVHPGTAECCTKEGLERK
LCMAALKHQPQEFPTYVEPTNDEICEAFRKDPKEYANQFMWEYSTNYGQAPLSLLVSYTK
SYLSMVGSCCTSASPTVCFLKERLQLKHLSLLTTLSNRVCSQYAAYGEKKSRLSNLIKLA
QKVPTADLEDVLPLAEDITNILSKCCESASEDCMAKELPEHTVKLCDNLSTKNSKFEDCC
QEKTAMDVFVCTYFMPAAQLPELPDVELPTNKDVCDPGNTKVMDKYTFELSRRTHLPEVF
LSKVLEPTLKSLGECCDVEDSTTCFNAKGPLLKKELSSFIDKGQELCADYSENTFTEYKK
KLAERLKAKLPDATPKELAKLVNKRSDFASNCCSINSPPLYCDSEIDAELKNIL
TALENS I
(SEQ ID NO: 8)
TCTTTTTCTCTTTAGCTCG
TALENS II
(SEQ ID NO: 9)
TACGTGCATCTCGACGAAA
SFP3
(SEQ ID NO: 10)
M K L L K L T G F I F F L F F L T E S L T L P T Q P R D I E N F N S T Q K F I E D
N I E Y I T I I A F A Q Y V Q E A T F E E M E K L V K D M V E Y K D R C M A D K T
L P E C S K L P N N V L Q E K I C A M E G L P Q K H N F S H C C S K V D A Q R R L
C F F Y N K K S D V G F L P P F P T L D P E E K C Q A Y E S N R E S L L N H F L Y
E V A R R N P F V F A P T L L T V A V H F E E V A K S C C E E Q N K V N C L Q T R
A I P V T Q Y L K A F S S Y Q K H V C G A L L K F G T K V V H F I Y I A I L S Q K
F P K I E F K E L I S L V E D V S S N Y D G C C E G D V V Q C I R D T S K V M N H
I C S K Q D S I S S K I K E C C E K K I P E R G Q C I I N S N K D D R P K D L S L
R E G K F T D S E N V C Q E R D A D P D T F F A K F T F E Y S R R H P D L S I P E
L L R I V Q I Y K D L L R N C C N T E N P P G C Y R Y A E D K F N E T T E K S L K
M V Q Q E C K H F Q N L G K Y Y L Q N A F L V A Y T K K A P Q L T S S E L M A I T
R K M A A T A A T C C Q L S E D K L L A C G E G A A D I I I G H L C I L H E M T P
V S D R V T Q C C T S S Y A N R R P C F S S L E V D E T Y V P K E F S D D K F T F
H S D L C Q A Q G V A L Q T M K Q E F L I N L V K H K P K I T E E Q L E A V I A D
F S G L L E K C C Q G Q E Q E V C F A E E G W K L I S K T R A A L G V
Amino Acid Sequence of Stabile9
(SEQ ID NO: 11)
M K W V T F I S L L F L F S S A Y S V F L D H E N A N K I L N R P K R Y N S G K L
E E F V Q G N L E R E C M E E K C S F E E A R E V F E N T E R T T E F W K Q Y V D
G D Q C E S N P C L N G G S C K D D I N S Y E C W C P F G F E G K N C E L D V T C
N I K N G R C E Q F C K N S A D N K V V C S C T E G Y R L A E N Q K S C E P A V P
F P C G R V S V S Q T S K L T R A E T V F P D V D Y V N S T E A E T I L D N I T Q
S T Q S F N D F T R V V G G E D A K P G Q F P W Q V V L N G K V D A F C G G S I V
N E K W I V T A A H C V E T G V K I T V V A G E H N I E E T E H T E Q K R N V I R
I I P H H N Y N A A I N K Y N H D I A L L E L D E P L V L N S Y V T P I C I A D K
E Y T N I F L K F G S G Y V S G W G R V F H K G R S A L V L Q Y L R V P L V D R A
T C L R S T K F T I Y N N M F C A G F H E G G R D S C Q G D S G G P H V T E V E G
T S F L T G I I S W G E E C A M K G K Y G I Y T K V S R Y V N W I K E K T K L T E
A A A K E A A A K E A A A K E A A A K E A A A K E K N K V C K E F S H L G K E D F
T S L S L V L Y S R K F P S G T F E Q V S Q L V K E V V S L T E A C V A E G A D P
D C Y D T R T S A L S A K S C E S N S P F P V H P G T A E C C T K E G L E R K L C
M A A L K H Q P Q E F P T Y V E P T N D E I C E A F R K D P K E Y A N Q F M W E Y
S T N Y G Q A P L S L L V S Y T K S Y L S M V G S C C T S A S P T V C F L K E R L
Q L K H L S L L T T L S N R V C S Q Y A A Y G E K K S R L S N L I K L A Q K V P T
A D L E D V L P L A E D I T N I L S K C C E S A S E D C M A K E L P E H T V K L C
D N Q D T K N S K F E D C C Q E K T A M D V F V C T Y F M P A A Q L P E L P D V E
L P T N K D V C D P G N T K V M D K Y T F E L S R R T H L P E V F L S K V L E P T
L K S L G E C C D V E D S T T C F N A K G P L L K K E L S S F I D K G Q E L C A D
Y S E N T F Y Y L Q N A F L V A Y T K K A P Q L T S S E L M A I T R K M A A T A A
T C C Q L S E D K L L A C G E G A A D I I I G H L C I L H E M T P V S D R V T Q C
C T S S Y A N R R P C F S S L E V D E T Y V P K E F S D D K F T F H S D L C Q A Q
G V A L Q T M K Q E F L I N L V K H K P K I T E E Q L E A V I A D F S G L L E K C
C Q G Q E Q E V C F A E E G W K L I S K T R A A L G V
Amino Acid Sequence of StabileBChE
(SEQ ID NO: 12)
M K W V T F I S L L F L F S S A Y S R G V F R R
S H T E D D I I I A T K N G K V R G M N L T V F G G T V T A F L G I P Y A Q P P L
G R L R F K K P Q S L T K W S D I W N A T K Y A N S C C Q N I D Q S F P G F H G S
E M W N P N T D L S E D C L Y L N V W I P A P K P K N A T V L I W I Y G G G F Q T
G T S S L H V Y D G K F L A R V E R V I V V S M N Y R V G A L G F L A L P G N P E
A P G N M G L F D Q Q L A L Q W V Q K N I A A F G G N P K S V T L F G E S A G A A
S V S L H L L S P G S H S L F T R A I L Q S G S F N A P W A V T S L Y E A R N R T
L N L A K L T G C S R E N E T E I I K C L R N K D P Q E I L L N E A F V V P Y G T
P L S V N F G P T V D G D F L T D M P D I L L E L G Q F K K T Q I L V G V N K D E
G T A F L V Y G A P G F S K D N N S I I T R K E F Q E G L K I F F P G V S E F G K
E S I L F H Y T D W V D D Q R P E N Y R E A L G D V V G D Y N F I C P A L E F T K
K F S E W G N N A F F Y Y F E H R S S K L P W P E W M G V M H G Y E I E F V F G L
P L E R R D N Y T K A E E I L S R S I V K R W A N F A K Y G N P N E T Q N N S T S
W P V F K S T E Q K Y L T L N T E S T R I M T K L R A Q Q C R F W T S F F P K V L
E M T G N I D E A E W E W K A G F H R W N N Y M M D W K N Q F N D Y T S K K E S C
V G L E A A A K E A A A K E A A A K E A A A K E A A A K E K N K V C K E F S H L G
K E D F T S L S L V L Y S R K F P S G T F E Q V S Q L V K E V V S L T E A C V A E
G A D P D C Y D T R T S A L S A K S C E S N S P F P V H P G T A E C C T K E G L E
R K L C M A A L K H Q P Q E F P T Y V E P T N D E I C E A F R K D P K E Y A N Q F
M W E Y S T N Y G Q A P L S L L V S Y T K S Y L S M V G S C C T S A S P T V C F L
K E R L Q L K H L S L L T T L S N R V C S Q Y A A Y G E K K S R L S N L I K L A Q
K V P T A D L E D V L P L A E D I T N I L S K C C E S A S E D C M A K E L P E H T
V K L C D N Q D T K N S K F E D C C Q E K T A M D V F V C T Y F M P A A Q L P E L
P D V E L P T N K D V C D P G N T K V M D K Y T F E L S R R T H L P E V F L S K V
L E P T L K S L G E C C D V E D S T T C F N A K G P L L K K E L S S F I D K G Q E
L C A D Y S E N T F Y Y L Q N A F L V A Y T K K A P Q L T S S E L M A I T R K M A
A T A A T C C Q L S E D K L L A C G E G A A D I I I G H L C I L H E M T P V S D R
V T Q C C T S S Y A N R R P C F S S L E V D E T Y V P K E F S D D K F T F H S D L
C Q A Q G V A L Q T M K Q E F L I N L V K H K P K I T E E Q L E A V I A D F S G L
L E K C C Q G Q E Q E V C F A E E G W K L I S K T R A A L G V
(Codon optimized human erythropoietin/albumin minigene construct)
SEQ ID NO: 13
          ttgggtagggaaggaagatttatgaaatatttaaaaaattattcttccttcgctttgtttttagacataatgttaa
atttattttgaaatttaaagcaacataaaagaacatgtgatttttctacttattgaaagagagaaaggaaaaaaatatgaa
acagggatggaaagaatcctatgcctggtgaaggtcaagggttctcataacctacagagaatttggggtcagcctgtccta
ttgtatattatggcaaagataatcatcatctcatagggtccattttcctctccatctctgcttaactgaagatcccatgag
atatactcacactgaatctaaatagcctatctcagggcttgaatcacatgtgggccacagcaggaatgggaacatggaatt
tctaagtcctatcttacttgttattgttgctatgtctttttcttagtttgcatctgaggcaacatcagctttttcagacag
aatggctttggaatagtaaaaaagacacagaagccctaaaatatgtatgtatgtatatgtgtgtgtgcatgcgtgagtact
tgtgtgtaaatttttcattatctataggtaaaagcacacttggaattagcaatagatgcaatttgggacttaactctttca
gtatgtcttatttctaagcaaagtatttagtttggttagtaattactaaacactgagaactaaattgcaaacaccaagaac
taaaatgttcaagtgggaaattacagttaaataccatggtaatgaataaaaggtacaaatcgtttaaactcttatgtaaaa
tttgataagatgttttacacaactttaatacattgacaaggtcttgtggagaaaacagttccagatggtaaatatacacaa
gggatttagtcaaacaattttttggcaagaatattatgaattttgtaatcggttggcagccaatgaaatacaaagatgagt
ctagttaataatctacaattattggttaaagaagtatattagtgctaatttccctccgtttgtcctagcttttctcttctg
tcaaccccacacgcctttggcacaatgggcgtgcacgaatgtcctgcctggctgtggctgctgctgagcctgctgtctctg
cctctgggactgcctgtgctgggagcccctcctagactgatctgcgacagccgggtgctggaaagatacctgctggaagcc
aaagaggccgagaacatcaccaccggctgcgccgagcactgcagcctgaacgagaatatcaccgtgcccgacaccaaagtg
aacttctacgcctggaagcggatggaagtgggccagcaggctgtggaagtgtggcagggactggccctgctgagcgaagct
gtgctgagaggacaggctctgctcgtgaacagcagccagccttgggagcctctgcagctgcacgtggacaaggccgtgtct
ggcctgagaagcctgaccacactgctgagagccctgggggcccagaaagaggccatctctccacctgatgccgcctctgcc
gcccctctgagaaccatcaccgccgacaccttcagaaagctgttccgggtgtacagcaacttcctgcggggcaagctgaag
ctgtacacaggcgaggcctgccggaccggcgatagataacccctctccctcccccccccctaacgttactggccgaagccg
cttggaataaggccggtgtgtgtttgtctatatgtgattttccaccatattgccgtcttttggcaatgtgagggcccggaa
acctggccctgtcttcttgacgagcattcctaggggtctttcccctctcgccaaaggaatgcaaggtctgttgaatgtcgt
gaaggaagcagttcctctggaagcttcttgaagacaaacaacgtctgtagcgaccctttgcaggcagcggaaccccccacc
tggcgacaggtgcctctgcggccaaaagccacgtgtataagatacacctgcaaaggcggcacaaccccagtgccacgttgt
gagttggatagttgtggaaagagtcaaatggctctcctcaagcgtagtcaacaaggggctgaaggatgcccagaaggtacc
ccattgtatgggaatctgatctggggcctcggtgcacatgctttacatgtgtttagtcgaggttaaaaaagctctaggccc
cccgaaccacggggacgtggttttcctttgaaaaacacgatgataagcttgccacaaccccgggataattcctgcagccaa
tatgggatcggccattgaacaagatggattgcacgcaggttctccggccgcttgggtggagaggctattcggctatgactg
ggcacaacagacaatcggctgctctgatgccgccgtgttccggctgtcagcgcaggggcgcccggttctttttgtcaagac
cgacctgtccggtgccctgaatgaactgcaggacgaggcagcgcggctatcgtggctggccacgacgggcgttccttgcgc
agctgtgctcgacgttgtcactgaagcgggaagggactggctgctattgggcgaagtgccggggcaggatctcctgtcatc
tcaccttgctcctgccgagaaagtatccatcatggctgatgcaatgcggcggctgcatacgcttgatccggctacctgccc
attcgaccaccaagcgaaacatcgcatcgagcgagcacgtactcggatggaagccggtcttgtcgatcaggatgatctgga
cgaagagcatcaggggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgactgtggccggctggg
tgtggcggaccgctatcaggacatagcgttggctacccgtgatattgctgaagagcttggcggcgaatgggctgaccgctt
cctcgtgctttacggtatcgccgctcccgattcgcagcgcatcgccttctatcgccttcttgacgagttcttctgacatca
catttaaaagcatctcaggtaactatattttgaattttttaaaaaagtaactataatagttattattaaaatagcaaagat
tgaccatttccaagagccatatagaccagcaccgaccactattctaaactatttatgtatgtaaatattagcttttaaaat
tctcaaaatagttgctgagttgggaaccactattatttctattttgtagatgagaaaatgaagataaacatcaaagcatag
attaagtaattttccaaagggtcaaaattcaaaattgaaaccaaagtttcagtgttgcccattgtcctgttctgacttata
tgatgcggtacacagagccatccaagtaagtgatggctcagcagtggaatactctgggaattaggctgaaccacatgaaag
agtgctttatagggcaaaaacagttgaatatcagtgatttcacatggttcaacctaatagttcaactcatcctttccattg
gagaatatgatggatctaccttctgtgaactttatagtgaagaatctgctattacatttccaatttgtcaacatgctgagc
tttaataggacttatcttcttatgacaacatttattggtgtgtccccttgcctagcccaacagaagaattcagcagccgta
agtctaggacaggcttaaattgttttcactggtgtaaattgcagaaagatgatctaagtaatttggcatttattttaatag
gtttgaaaaacacatgccattttacaaataagacttatatttgtccttttgtttttcagcctaccatgagaataagagaaa
gaaaatgaagatcaaaagcttattcatctgtttttctttttcgttggtgtaaagccaacaccctgtctaaaaaacataaat
ttctttaatcattttgcctcttttctctgtgcttcaattaataaaaaatggaaagaatct
(Codon optimized human stem cell factor/albumin
minigene construct)
SEQ ID NO: 14
          ttgggtagggaaggaagatttatgaaatatttaaaaaattattcttccttcgctttgtttttagacataatgttaa
atttattttgaaatttaaagcaacataaaagaacatgtgatttttctacttattgaaagagagaaaggaaaaaaatatgaa
acagggatggaaagaatcctatgcctggtgaaggtcaagggttctcataacctacagagaatttggggtcagcctgtccta
ttgtatattatggcaaagataatcatcatctcatttgggtccattttcctctccatctctgcttaactgaagatcccatga
gatatactcacactgaatctaaatagcctatctcagggcttgaatcacatgtgggccacagcaggaatgggaacatggaat
ttctaagtcctatcttacttgttattgttgctatgtctttttcttagtttgcatctgaggcaacatcagctttttcagaca
gaatggctttggaatagtaaaaaagacacagaagccctaaaatatgtatgtatgtatatgtgtgtgtgcatgcgtgagtac
ttgtgtgtaaatttttcattatctataggtaaaagcacacttggaattagcaatagatgcaatttgggacttaactctttc
agtatgtcttatttctaagcaaagtatttagtttggttagtaattactaaacactgagaactaaattgcaaacaccaagaa
ctaaaatgttcaagtgggaaattacagttaaataccatggtaatgaataaaaggtacaaatcgtttaaactcttatgtaaa
atttgataagatgttttacacaactttaatacattgacaaggtcttgtggagaaaacagttccagatggtaaatatacaca
agggatttagtcaaacaattttttggcaagaatattatgaattttgtaatcggttggcagccaatgaaatacaaagatgag
tctagttaataatctacaattattggttaaagaagtatattagtgctaatttccctccgtttgtcctagcttttctcttct
gtcaaccccacacgcctttggcacaatgaagaaaacccagacctggatcctgacctgcatctacctgcagctgctgctgtt
caaccccctcgtgaaaaccgagggcatctgccggaacagagtgaccaacaacgtgaaggacgtgaccaagctggtggccaa
cctgcccaaggactacatgatcaccctgaaatacgtgcccggcatggacgtgctgcccagccactgttggatcagcgagat
ggtggtgcagctgagcgacagcctgaccgacctgctggacaagttcagcaacatcagcgagggcctgagcaactacagcat
catcgataagctcgtgaacatcgtggacgacctggtggaatgcgtgaaagagaacagctccaaggacctgaagaagtcctt
caagagccccgagcccagactgttcacccccgaggaattcttccggatcttcaaccggtccatcgacgccttcaaggactt
cgtggtggccagcgagacaagcgactgcgtggtgtctagcaccctgtcccccgagaaggacagcagagtgtccgtgacaaa
gcccttcatgctgccccctgtggccgcctaacccctctccctcccccccccctaacgttactggccgaagccgcttggaat
aaggccggtgtgtgtttgtctatatgtgattttccaccatattgccgtcttttggcaatgtgagggcccggaaacctggcc
ctgtcttcttgacgagcattcctaggggtctttcccctctcgccaaaggaatgcaaggtctgttgaatgtcgtgaaggaag
cagttcctctggaagcttcttgaagacaaacaacgtctgtagcgaccctttgcaggcagcggaaccccccacctggcgaca
ggtgcctctgcggccaaaagccacgtgtataagatacacctgcaaaggcggcacaaccccagtgccacgttgtgagttgga
tagttgtggaaagagtcaaatggctctcctcaagcgtagtcaacaaggggctgaaggatgcccagaaggtaccccattgta
tgggaatctgatctggggcctcggtgcacatgctttacatgtgtttagtcgaggttaaaaaagctctaggccccccgaacc
acggggacgtggttttcctttgaaaaacacgatgataagcttgccacaaccccgggataattcctgcagccaatatgggat
cggccattgaacaagatggattgcacgcaggttctccggccgcttgggtggagaggctattcggctatgactgggcacaac
agacaatcggctgctctgatgccgccgtgttccggctgtcagcgcaggggcgcccggttctttttgtcaagaccgacctgt
ccggtgccctgaatgaactgcaggacgaggcagcgcggctatcgtggctggccacgacgggcgttccttgcgcagctgtgc
tcgacgttgtcactgaagcgggaagggactggctgctattgggcgaagtgccggggcaggatctcctgtcatctcaccttg
ctcctgccgagaaagtatccatcatggctgatgcaatgcggcggctgcatacgcttgatccggctacctgcccattcgacc
accaagcgaaacatcgcatcgagcgagcacgtactcggatggaagccggtcttgtcgatcaggatgatctggacgaagagc
atcaggggctcgcgccagccgaactgttcgccaggctcaaggcgcgcatgcccgacggcgaggatctcgtcgtgacccatg
gcgatgcctgcttgccgaatatcatggtggaaaatggccgcttttctggattcatcgactgtggccggctgggtgtggcgg
accgctatcaggacatagcgttggctacccgtgatattgctgaagagcttggcggcgaatgggctgaccgcttcctcgtgc
tttacggtatcgccgctcccgattcgcagcgcatcgccttctatcgccttcttgacgagttcttctgacatcacatttaaa
agcatctcaggtaactatattttgaattttttaaaaaagtaactataatagttattattaaaatagcaaagattgaccatt
tccaagagccatatagaccagcaccgaccactattctaaactatttatgtatgtaaatattagcttttaaaattctcaaaa
tagttgctgagttgggaaccactattatttctattttgtagatgagaaaatgaagataaacatcaaagcatagattaagta
attttccaaagggtcaaaattcaaaattgaaaccaaagtttcagtgttgcccattgtcctgttctgacttatatgatgcgg
tacacagagccatccaagtaagtgatggctcagcagtggaatactctgggaattaggctgaaccacatgaaagagtgcttt
atagggcaaaaacagttgaatatcagtgatttcacatggttcaacctaatagttcaactcatcctttccattggagaatat
gatggatctaccttctgtgaactttatagtgaagaatctgctattacatttccaatttgtcaacatgctgagctttaatag
gacttatcttcttatgacaacatttattggtgtgtccccttgcctagcccaacagaagaattcagcagccgtaagtctagg
acaggcttaaattgttttcactggtgtaaattgcagaaagatgatctaagtaatttggcatttattttaataggtttgaaa
aacacatgccattttacaaataagacttatatttgtccttttgtttttcagcctaccatgagaataagagaaagaaaatga
agatcaaaagcttattcatctgtttttctttttcgttggtgtaaagccaacaccctgtctaaaaaacataaatttctttaa
tcattttgcctcttttctctgtgcttcaattaataaaaaatggaaagaatct
(Codon optimized human Factor IX/albumin minigene construct)
SEQ ID NO: 15
          ttgggtagggaaggaagatttatgaaatatttaaaaaattattcttccttcgctttgtttttagacataatgttaa
atttattttgaaatttaaagcaacataaaagaacatgtgatttttctacttattgaaagagagaaaggaaaaaaatatgaa
acagggatggaaagaatcctatgcctggtgaaggtcaagggttctcataacctacagagaatttggggtcagcctgtccta
ttgtatattatggcaaagataatcatcatctcatttgggtccattttcctaccatctctgcttaactgaagatcccatgag
atatactcacactgaatctaaatagcctatctcagggcttgaatcacatgtgggccacagcaggaatgggaacatggaatt
tctaagtcctatcttacttgttattgttgctatgtctttttcttagtttgcatctgaggcaacatcagctttttcagacag
aatggctttggaatagtaaaaaagacacagaagccctaaaatatgtatgtatgtatatgtgtgtgtgcatgcgtgagtact
tgtgtgtaaatttttcattatctataggtaaaagcacacttggaattagcaatagatgcaatttgggacttaactctttca
gtatgtcttatttctaagcaaagtatttagtttggttagtaattactaaacactgagaactaaattgcaaacaccaagaac
taaaatgttcaagtgggaaattacagttaaataccatggtaatgaataaaaggtacaaatcgtttaaactcttatgtaaaa
tttgataagatgttttacacaactttaatacattgacaaggtcttgtggagaaaacagttccagatggtaaatatacacaa
gggatttagtcaaacaattttttggcaagaatattatgaattttgtaatcggttggcagccaatgaaatacaaagatgagt
ctagttaataatctacaattattggttaaagaagtatattagtgctaatttccctccgtttgtcctagcttttctcttctg
tcaaccccacacgcctttggcacaatgcagcgcgtgaacatgattatggccgagagccctggcctgatcaccatctgcctg
ctgggctacctgctgagcgccgagtgcaccgtgtttctggaccacgagaacgccaacaagatcctgaaccggcccaagcgg
tacaacagcggcaagctggaagagttcgtgcagggcaacctggaacgcgagtgcatggaagagaagtgcagcttcgaagag
gccagagaggtgttcgagaacaccgagcggaccaccgagttctggaagcagtacgtggacggcgaccagtgcgagagcaac
ccctgtctgaatggcggcagctgcaaggacgacatcaacagctacgagtgctggtgccccttcggcttcgagggcaagaac
tgcgagctggacgtgacctgcaacatcaagaacggcagatgcgagcagttctgcaagaacagcgccgacaacaaggtcgtg
tgctcctgcaccgagggctacagactggccgagaaccagaagtcctgcgagcccgccgtgcctttccatgtggaagagtgt
ccgtgtcccagaccagcaagctgaccagagccgagacagtgttccccgacgtggactacgtgaacagcaccgaggccgaga
caatcctggacaacatcacccagagcacccagtccttcaacgacttcaccagagtcgtgggcggcgaggatgccaagcctg
gacagttcccgtggcaggtggtgctgaacggaaaggtggacgccttttgcggcggcagcatcgtgaacgagaagtggatcg
tgacagccgcccactgcgtggaaaccggcgtgaagattacagtggtggccggcgagcacaacatcgaggaaaccgagcaca
cagagcagaaacggaacgtgatcagaatcatcccccaccacaactacaacgccgccatcaacaagtacaaccacgatatcg
ccctgctggaactggacgagcccctggtgctgaatagctacgtgacccccatctgtatcgccgacaaagagtacaccaaca
tctttctgaagttcggcagcggctacgtgtccggctggggcagagtgtttcacaagggcagatccgctctggtgctgcagt
acctgagagtgcctctggtggaccgggccacctgtctgagaagcaccaagttcaccatctacaacaacatgttctgcgccg
gctttcacgagggcggcagagatagctgtcagggcgattctggcggccctcacgtgacagaggtggaaggcaccagctttc
tgaccggcatcatcagctggggcgaggaatgcgccatgaaggggaagtacggcatctacaccaaggtgtccagatacgtga
actggatcaaagaaaagaccaagctgacataacccctctccctcccccccccctaacgttactggccgaagccgcttggaa
taaggccggtgtgtgtttgtctatatgtgattttccaccatattgccgtcttttggcaatgtgagggcccggaaacctggc
cctgtcttcttgacgagcattcctaggggtctttcccctctcgccaaaggaatgcaaggtctgttgaatgtcgtgaaggaa
gcagttcctctggaagcttcttgaagacaaacaacgtctgtagcgaccctttgcaggcagcggaaccccccacctggcgac
aggtgcctctgcggccaaaagccacgtgtataagatacacctgcaaaggcggcacaaccccagtgccacgttgtgagttgg
atagttgtggaaagagtcaaatggctctcctcaagcgtagtcaacaaggggctgaaggatgcccagaaggtaccccattgt
atgggaatctgatctggggcctcggtgcacatgctttacatgtgtttagtcgaggttaaaaaagctctaggccccccgaac
cacggggacgtggttttcctttgaaaaacacgatgataagcttgccacaaccccgggataattcctgcagccaatatggga
tcggccattgaacaagatggattgcacgcaggttctccggccgcttgggtggagaggctattcggctatgactgggcacaa
cagacaatcggctgctctgatgccgccgtgttccggctgtcagcgcaggggcgcccggttctttttgtcaagaccgacctg
tccggtgccctgaatgaactgcaggacgaggcagcgcggctatcgtggctggccacgacgggcgttccttgcgcagctgtg
ctcgacgttgtcactgaagcgggaagggactggctgctattgggcgaagtgccggggcaggatctcctgtcatctcacctt
gctcctgccgagaaagtatccatcatggctgatgcaatgcggcggctgcatacgcttgatccggctacctgcccattcgac
caccaagcgaaacatcgcatcgagcgagcacgtactcggatggaagccggtcttgtcgatcaggatgatctggacgaagag
catcaggggctcgcgccagccgaactgttcgccaggctcaaggcgcgcatgcccgacggcgaggatctcgtcgtgacccat
ggcgatgcctgcttgccgaatatcatggtggaaaatggccgcttttctggattcatcgactgtggccggctgggtgtggcg
gaccgctatcaggacatagcgttggctacccgtgatattgctgaagagcttggcggcgaatgggctgaccgcttcctcgtg
ctttacggtatcgccgctcccgattcgcagcgcatcgccttctatcgccttcttgacgagttcttctgacatcacatttaa
aagcatctcaggtaactatattttgaattttttaaaaaagtaactataatagttattattaaaatagcaaagattgaccat
ttccaagagccatatagaccagcaccgaccactattctaaactatttatgtatgtaaatattagcttttaaaattctcaaa
atagttgctgagttgggaaccactattatttctattttgtagatgagaaaatgaagataaacatcaaagcatagattaagt
aattttccaaagggtcaaaattcaaaattgaaaccaaagtttcagtgttgcccattgtcctgttctgacttatatgatgcg
gtacacagagccatccaagtaagtgatggctcagcagtggaatactctgggaattaggctgaaccacatgaaagagtgctt
tatagggcaaaaacagttgaatatcagtgatttcacatggttcaacctaatagttcaactcatcctttccattggagaata
tgatggatctaccttctgtgaactttatagtgaagaatctgctattacatttccaatttgtcaacatgctgagctttaata
ggacttatcttcttatgacaacatttattggtgtgtccccttgcctagcccaacagaagaattcagcagccgtaagtctag
gacaggcttaaattgttttcactggtgtaaattgcagaaagatgatctaagtaatttggcatttattttaataggtttgaa
aaacacatgccattttacaaataagacttatatttgtccttttgtttttcagcctaccatgagaataagagaaagaaaatg
aagatcaaaagcttattcatctgtttttctttttcgttggtgtaaagccaacaccctgtctaaaaaacataaatttcttta
atcattttgcctcttttctctgtgcttcaattaataaaaaatggaaagaat
(Codon optimized human Interleukin 3/albumin minigene construct)
SEQ ID NO: 16
          ttgggtagggaaggaagatttatgaaatatttaaaaaattattcttccttcgctttgtttttagacataatgttaa
atttattttgaaatttaaagcaacataaaagaacatgtgatttttctacttattgaaagagagaaaggaaaaaaatatgaa
acagggatggaaagaatcctatgcctggtgaaggtcaagggttctcataacctacagagaatttggggtcagcctgtccta
ttgtatattatggcaaagataatcatcatctcatttgggtccattttcctctccatctctgcttaactgaagatcccatga
gatatactcacactgaatctaaatagcctatctcagggcttgaatcacatgtgggccacagcaggaatgggaacatggaat
ttctaagtcctatcttacttgttattgttgctatgtctttttcttagtttgcatctgaggcaacatcagctttttcagaca
gaatggctttggaatagtaaaaaagacacagaagccctaaaatatgtatgtatgtatatgtgtgtgtgcatgcgtgagtac
ttgtgtgtaaatttttcattatctataggtaaaagcacacttggaattagcaatagatgcaatttgggacttaactctttc
agtatgtcttatttctaagcaaagtatttagtttggttagtaattactaaacactgagaactaaattgcaaacaccaagaa
ctaaaatgttcaagtgggaaattacagttaaataccatggtaatgaataaaaggtacaaatcgtttaaactcttatgtaaa
atttgataagatgttttacacaactttaatacattgacaaggtcttgtggagaaaacagttccagatggtaaatatacaca
agggatttagtcaaacaattttttggcaagaatattatgaattttgtaatcggttggcagccaatgaaatacaaagatgag
tctagttaataatctacaattattggttaaagaagtatattagtgctaatttccctccgtttgtcctagcttttctcttct
gtcaaccccacacgcctttggcacaatgagcagactgcccgtgctcctgctgctgcagctgctcgtgcggcctggactgca
ggctcctatgacccagaccacccccctgaaaaccagctgggtcaactgcagcaacatgatcgacgagatcatcacccacct
gaagcagccccccctgcccctgctggacttcaacaacctgaacggcgaggaccaggacatcctgatggaaaacaacctgcg
gaggcccaacctggaagccttcaacagagccgtgaagtccctgcagaacgccagcgccatcgagagcatcctgaagaacct
gctgccctgcctgcctctggccacagccgctcctacaagacaccccatccacatcaaggacggcgactggaacgagttccg
gcggaagctgaccttctacctgaaaacactggaaaacgcccaggcccagcagaccacactgagcctggccatcttctaacc
cctctccctcccccccccctaacgttactggccgaagccgcttggaataaggccggtgtgtgtttgtctatatgtgatttt
ccaccatattgccgtctttggcaatgtgagggcccggaaacctggccctgtcttcttgacgagcattcctaggggtctttc
ccctctcgccaaaggaatgcaaggtctgttgaatgtcgtgaaggaagcagttcctctggaagcttcttgaagacaaacaac
gtctgtagcgacccctttgcaggcagcggaaccccccacctggcgacaggtgcctctgcggccaaaagccacgtgtataag
atacacctgcaaaggcggcacaaccccagtgccacgttgtgagttggatagttgtggaaagagtcaaatggctctcctcaa
gcgtagtcaacaaggggctgaaggatgcccagaaggtaccccattgtatgggaatctgatctggggcctcggtgcacatgc
tttacatgtgtttagtcgaggttaaaaaagctctaggccccccgaaccacggggacgtggttttcctttgaaaaacacgat
gataagcttgccacaaccccgggataattcctgcagccaatatgggatcggccattgaacaagatggattgcacgcaggtt
ctccggccgcttgggtggagaggctattcggctatgactgggcacaacagacaatcggctgctctgatgccgccgtgttcc
ggctgtcagcgcaggggcgcccggttctttttgtcaagaccgacctgtccggtgccctgaatgaactgcaggacgaggcag
cgcggctatcgtggctggccacgacgggcgttccttgcgcagctgtgctcgacgttgtcactgaagcgggaagggactggc
tgctattgggcgaagtgccggggcaggatctcctgtcatctcaccttgctcctgccgagaaagtatccatcatggctgatg
caatgcggcggctgcatacgcttgatccggctacctgcccattcgaccaccaagcgaaacatcgcatcgagcgagcacgta
ctcggatggaagccggtcttgtcgatcaggatgatctggacgaagagcatcaggggctcgcgccagccgaactgttcgcca
ggctcaaggcgcgcatgcccgacggcgaggatctcgtcgtgacccatggcgatgcctgcttgccgaatatcatggtggaaa
atggccgcttttctggattcatcgactgtggccggctgggtgtggcggaccgctatcaggacatagcgttggctacccgtg
atattgctgaagagcttggcggcgaatgggctgaccgcttcctcgtgctttacggtatcgccgctcccgattcgcagcgca
tcgccttctatcgccttcttgacgagttcttctgacatcacatttaaaagcatctcaggtaactatattttgaatttttta
aaaaagtaactataatagttattattaaaatagcaaagattgaccatttccaagagccatatagaccagcaccgaccacta
ttctaaactatttatgtatgtaaatattagcttttaaaattctcaaaatagttgctgagttgggaaccactattatttcta
ttttgtagatgagaaaatgaagataaacatcaaagcatagattaagtaattttccaaagggtcaaaattcaaaattgaaac
caaagtttcagtgttgcccattgtcctgttctgacttatatgatgcggtacacagagccatccaagtaagtgatggctcag
cagtggaatactctgggaattaggctgaaccacatgaaagagtgctttatagggcaaaaacagttgaatatcagtgatttc
acatggttcaacctaatagttcaactcatcctttccattggagaatatgatggatctaccttctgtgaactttatagtgaa
gaatctgctattacatttccaatttgtcaacatgctgagctttaataggacttatcttcttatgacaacatttattggtgt
gtccccttgcctagcccaacagaagaattcagcagccgtaagtctaggacaggcttaaattgttttcactggtgtaaattg
cagaaagatgatctaagtaatttggcatttattttaataggtttgaaaaacacatgccattttacaaataagacttatatt
tgtccttttgtttttcagcctaccatgagaataagagaaagaaaatgaagatcaaaagcttattcatctgtttttcttttt
cgttggtgtaaagccaacaccctgtctaaaaaacataaatttctttaatcattttgcctcttttctctgtgcttcaattaa
taaaaaatggaaagaatct
(Codon optimized human Thrombopoietin/albumin minigene construct)
SEQ ID NO: 17
          ttgggtagggaaggaagatttatgaaatatttaaaaaattattcttccttcgctttgtttttagacataatgttaa
atttattttgaaatttaaagcaacataaaagaacatgtgatttttctacttattgaaagagagaaaggaaaaaaatatgaa
acagggatggaaagaatcctatgcctggtgaaggtcaagggttctcataacctacagagaatttggggtcagcctgtccta
ttgtatattatggcaaagataatcatcatctcatttgggtccattttcctctccatctctgcttaactgaagatcccatga
gatatactcacactgaatctaaatagcctatctcagggcttgaatcacatgtgggccacagcaggaatgggaacatggaat
ttctaagtcctatcttacttgttattgttgctatgtctttttcttagtttgcatctgaggcaacatcagctttttcagaca
gaatggctttggaatagtaaaaaagacacagaagccctaaaatatgtatgtatgtatatgtgtgtgtgcatgcgtgagtac
ttgtgtgtaaatttttcattatctataggtaaaagcacacttggaattagcaatagatgcaatttgggacttaactctttc
agtatgtcttatttctaagcaaagtatttagtttggttagtaattactaaacactgagaactaaattgcaaacaccaagaa
ctaaaatgttcaagtgggaaattacagttaaataccatggtaatgaataaaaggtacaaatcgtttaaactcttatgtaaa
atttgataagatgttttacacaactttaatacattgacaaggtcttgtggagaaaacagttccagatggtaaatatacaca
agggatttagtcaaacaattttttggcaagaatattatgaattttgtaatcggttggcagccaatgaaatacaaagatgag
tctagttaataatctacaattattggttaaagaagtatattagtgctaatttccctccgtttgtcctagcttttctcttct
gtcaaccccacacgcctttggcacaatggaactgaccgagctgctgctggtcgtgatgctgctgctgaccgccagactgac
cctgtctagccctgcccctcctgcctgcgatctgagagtgctgagcaagctgctgcgggacagccacgtgctgcacagcag
actgagccagtgccctgaggtgcaccctctgcctacacctgtgctgctgcctgccgtggatttcagcctgggcgagtggaa
aacccagatggaagagacaaaggcccaggacatcctgggagccgtgaccctgctgctggaaggcgtgatggctgccagagg
acagctgggccctacctgtctgtcctctctgctgggccagctgtctggacaagtgcggctgctgctgggagccctgcagtc
tctgctgggaacacagctgcctccccagggcagaaccaccgcccacaaggaccccaacgccatcttcctgagcttccagca
tctgctgagaggcaaagtgcggttcctgatgctcgtgggcggcagcacactgtgcgtgcggagagcacctcctaccacagc
cgtgcctagcagaaccagcctggtgctgaccctgaacgagctgcccaacagaacctccggcctgctggaaacaaacttcac
cgccagcgccaggaccacaggctctggactgctgaagtggcagcagggcttccgggccaagattcctggcctgctgaacca
gaccagcagaagcctggaccagatccccggctacctgaaccggatccacgaactgctgaacggcaccagaggcctgttccc
aggcccctccagaagaacactgggcgctcccgatatcagcagcggcacctctgataccggcagcctgccccctaatctgca
gcctggctacagccctagccctacccaccctccaaccggccagtacaccctgttccctctgccacctaccctgcccacacc
agtggtgcagctgcatcctctgctgcccgatcctagcgcccctacccctacaccaacaagccccctgctgaataccagcta
cacccacagccagaacctgagccaggaaggctaacccctctccctcccccccccctaacgttactggccgaagccgcttgg
aataaggccggtgtgtgtttgtctatatgtgattttccaccatattgccgtcttttggcaatgtgagggcccggaaacctg
gccctgtcttcttgacgagcattcctaggggtctttcccctctcgccaaaggaatgcaaggtctgttgaatgtcgtgaagg
aagcagttcctctggaagcttcttgaagacaaacaacgtctgtagcgaccctttgcaggcagcggaaccccccacctggcg
acaggtgcctctgcggccaaaagccacgtgtataagatacacctgcaaaggcggcacaaccccagtgccacgttgtgagtt
ggatagttgtggaaagagtcaaatggctctcctcaagcgtagtcaacaaggggctgaaggatgcccagaaggtaccccatt
gtatgggaatctgatctggggcctcggtgcacatgctttacatgtgtttagtcgaggttaaaaaagctctaggccccccga
accacggggacgtggttttcctttgaaaaacacgatgataagcttgccacaaccccgggataattcctgcagccaatatgg
gatcggccattgaacaagatggattgcacgcaggttctccggccgcttgggtggagaggctattcggctatgactgggcac
aacagacaatcggctgctctgatgccgccgtgttccggctgtcagcgcaggggcgcccggttctttttgtcaagaccgacc
tgtccggtgccctgaatgaactgcaggacgaggcagcgcggctatcgtggctggccacgacgggcgttccttgcgcagctg
tgctcgacgttgtcactgaagcgggaagggactggctgctattgggcgaagtgccggggcaggatctcctgtcatctcacc
ttgctcctgccgagaaagtatccatcatggctgatgcaatgcggcggctgcatacgcttgatccggctacctgcccattcg
accaccaagcgaaacatcgcatcgagcgagcacgtactcggatggaagccggtcttgtcgatcaggatgatctggacgaag
agcatcaggggctcgcgccagccgaactgttcgccaggctcaaggcgcgcatgcccgacggcgaggatctcgtcgtgaccc
atggcgatgcctgcttgccgaatatcatggtggaaaatggccgcttttctggattcatcgactgtggccggctgggtgtgg
cggaccgctatcaggacatagcgttggctacccgtgatattgctgaagagcttggcggcgaatgggctgaccgcttcctcg
tgctttacggtatcgccgctcccgattcgcagcgcatcgccttctatcgccttcttgacgagttcttctgacatcacattt
aaaagcatctcaggtaactatattttgaattttttaaaaaagtaactataatagttattattaaaatagcaaagattgacc
atttccaagagccatatagaccagcaccgaccactattctaaactatttatgtatgtaaatattagcttttaaaattctca
aaatagttgctgagttgggaaccactattatttctattttgtagatgagaaaatgaagataaacatcaaagcatagattaa
gtaattttccaaagggtcaaaattcaaaattgaaaccaaagtttcagtgttgcccattgtcctgttctgacttatatgatg
cggtacacagagccatccaagtaagtgatggctcagcagtggaatactctgggaattaggctgaaccacatgaaagagtgc
tttatagggcaaaaacagttgaatatcagtgatttcacatggttcaacctaatagttcaactcatcctttccattggagaa
tatgatggatctaccttctgtgaactttatagtgaagaatctgctattacatttccaatttgtcaacatgctgagctttaa
taggacttatcttcttatgacaacatttattggtgtgtccccttgcctagcccaacagaagaattcagcagccgtaagtct
aggacaggcttaaattgttttcactggtgtaaattgcagaaagatgatctaagtaatttggcatttattttaataggtttg
aaaaacacatgccattttacaaataagacttatatttgtccttttgtttttcagcctaccatgagaataagagaaagaaaa
tgaagatcaaaagcttattcatctgtttttctttttcgttggtgtaaagccaacaccctgtctaaaaaacataaatttctt
taatcattttgcctcttttctctgtgcttcaattaataaaaaatggaaagaatct
(Codon optimized human Factor IX cDNA)
SEQ ID NO: 18
          ATGCAGCGCGTGAACATGATTATGGCCGAGAGCCCTGGCCTGATCACCATCTGCCTGCTGGGC
TACCTGCTGAGCGCCGAGTGCACCGTGTTTCTGGACCACGAGAACGCCAACAAGATCCTGAACCGGCC
CAAGCGGTACAACAGCGGCAAGCTGGAAGAGTTCGTGCAGGGCAACCTGGAACGCGAGTGCATGGAA
GAGAAGTGCAGCTTCGAAGAGGCCAGAGAGGTGTTCGAGAACACCGAGCGGACCACCGAGTTCTGGA
AGCAGTACGTGGACGGCGACCAGTGCGAGAGCAACCCCTGTCTGAATGGCGGCAGCTGCAAGGACGA
CATCAACAGCTACGAGTGCTGGTGCCCCTTCGGCTTCGAGGGCAAGAACTGCGAGCTGGACGTGACCT
GCAACATCAAGAACGGCAGATGCGAGCAGTTCTGCAAGAACAGCGCCGACAACAAGGTCGTGTGCTC
CTGCACCGAGGGCTACAGACTGGCCGAGAACCAGAAGTCCTGCGAGCCCGCCGTGCCTTTCCCATGTG
GAAGAGTGTCCGTGTCCCAGACCAGCAAGCTGACCAGAGCCGAGACAGTGTTCCCCGACGTGGACTA
CGTGAACAGCACCGAGGCCGAGACAATCCTGGACAACATCACCCAGAGCACCCAGTCCTTCAACGAC
TTCACCAGAGTCGTGGGCGGCGAGGATGCCAAGCCTGGACAGTTCCCGTGGCAGGTGGTGCTGAACG
GAAAGGTGGACGCCTTTTGCGGCGGCAGCATCGTGAACGAGAAGTGGATCGTGACAGCCGCCCACTG
CGTGGAAACCGGCGTGAAGATTACAGTGGTGGCCGGCGAGCACAACATCGAGGAAACCGAGCACACA
GAGCAGAAACGGAACGTGATCAGAATCATCCCCCACCACAACTACAACGCCGCCATCAACAAGTACA
ACCACGATATCGCCCTGCTGGAACTGGACGAGCCCCTGGTGCTGAATAGCTACGTGACCCCCATCTGT
ATCGCCGACAAAGAGTACACCAACATCTTTCTGAAGTTCGGCAGCGGCTACGTGTCCGGCTGGGGCAG
AGTGTTTCACAAGGGCAGATCCGCTCTGGTGCTGCAGTACCTGAGAGTGCCTCTGGTGGACCGGGCCA
CCTGTCTGAGAAGCACCAAGTTCACCATCTACAACAACATGTTCTGCGCCGGCTTTCACGAGGGCGGC
AGAGATAGCTGTCAGGGCGATTCTGGCGGCCCTCACGTGACAGAGGTGGAAGGCACCAGCTTTCTGAC
CGGCATCATCAGCTGGGGCGAGGAATGCGCCATGAAGGGGAAGTACGGCATCTACACCAAGGTGTCC
AGATACGTGAACTGGATCAAAGAAAAGACCAAGCTGACATAA
(Codon optimized human Erythropoietin cDNA)
SEQ ID NO: 19
          ATGGGCGTGCACGAATGTCCTGCCTGGCTGTGGCTGCTGCTGAGCCTGCTGTCTCTGCCTCTG
GGACTGCCTGTGCTGGGAGCCCCTCCTAGACTGATCTGCGACAGCCGGGTGCTGGAAAGATACCTGCT
GGAAGCCAAAGAGGCCGAGAACATCACCACCGGCTGCGCCGAGCACTGCAGCCTGAACGAGAATATC
ACCGTGCCCGACACCAAAGTGAACTTCTACGCCTGGAAGCGGATGGAAGTGGGCCAGCAGGCTGTGG
AAGTGTGGCAGGGACTGGCCCTGCTGAGCGAAGCTGTGCTGAGAGGACAGGCTCTGCTCGTGAACAG
CAGCCAGCCTTGGGAGCCTCTGCAGCTGCACGTGGACAAGGCCGTGTCTGGCCTGAGAAGCCTGACCA
CACTGCTGAGAGCCCTGGGGGCCCAGAAAGAGGCCATCTCTCCACCTGATGCCGCCTCTGCCGCCCCT
CTGAGAACCATCACCGCCGACACCTTCAGAAAGCTGTTCCGGGTGTACAGCAACTTCCTGCGGGGCAA
GCTGAAGCTGTACACAGGCGAGGCCTGCCGGACCGGCGATAGATAA
(Codon optimized human Interleukin 3 cDNA)
SEQ ID NO: 20
          ATGAGCAGACTGCCCGTGCTCCTGCTGCTGCAGCTGCTCGTGCGGCCTGGACTGCAGGCTCCT
ATGACCCAGACCACCCCCCTGAAAACCAGCTGGGTCAACTGCAGCAACATGATCGACGAGATCATCA
CCCACCTGAAGCAGCCCCCCCTGCCCCTGCTGGACTTCAACAACCTGAACGGCGAGGACCAGGACATC
CTGATGGAAAACAACCTGCGGAGGCCCAACCTGGAAGCCTTCAACAGAGCCGTGAAGTCCCTGCAGA
ACGCCAGCGCCATCGAGAGCATCCTGAAGAACCTGCTGCCCTGCCTGCCTCTGGCCACAGCCGCTCCT
ACAAGACACCCCATCCACATCAAGGACGGCGACTGGAACGAGTTCCGGCGGAAGCTGACCTTCTACC
TGAAAACACTGGAAAACGCCCAGGCCCAGCAGACCACACTGAGCCTGGCCATCTTCTAA
(Codon optimized human Stem Cell Factor cDNA)
SEQ ID NO: 21
          ATGAAGAAAACCCAGACCTGGATCCTGACCTGCATCTACCTGCAGCTGCTGCTGTTCAACCCC
CTCGTGAAAACCGAGGGCATCTGCCGGAACAGAGTGACCAACAACGTGAAGGACGTGACCAAGCTGG
TGGCCAACCTGCCCAAGGACTACATGATCACCCTGAAATACGTGCCCGGCATGGACGTGCTGCCCAGC
CACTGTTGGATCAGCGAGATGGTGGTGCAGCTGAGCGACAGCCTGACCGACCTGCTGGACAAGTTCAG
CAACATCAGCGAGGGCCTGAGCAACTACAGCATCATCGATAAGCTCGTGAACATCGTGGACGACCTG
GTGGAATGCGTGAAAGAGAACAGCTCCAAGGACCTGAAGAAGTCCTTCAAGAGCCCCGAGCCCAGAC
TGTTCACCCCCGAGGAATTCTTCCGGATCTTCAACCGGTCCATCGACGCCTTCAAGGACTTCGTGGTGG
CCAGCGAGACAAGCGACTGCGTGGTGTCTAGCACCCTGTCCCCCGAGAAGGACAGCAGAGTGTCCGT
GACAAAGCCCTTCATGCTGCCCCCTGTGGCCGCCTAA
(Codon optimized human Thrombopoietin cDNA)
SEQ ID NO: 22
          ATGGAACTGACCGAGCTGCTGCTGGTCGTGATGCTGCTGCTGACCGCCAGACTGACCCTGTCT
AGCCCTGCCCCTCCTGCCTGCGATCTGAGAGTGCTGAGCAAGCTGCTGCGGGACAGCCACGTGCTGCA
CAGCAGACTGAGCCAGTGCCCTGAGGTGCACCCTCTGCCTACACCTGTGCTGCTGCCTGCCGTGGATTT
CAGCCTGGGCGAGTGGAAAACCCAGATGGAAGAGACAAAGGCCCAGGACATCCTGGGAGCCGTGACC
CTGCTGCTGGAAGGCGTGATGGCTGCCAGAGGACAGCTGGGCCCTACCTGTCTGTCCTCTCTGCTGGG
CCAGCTGTCTGGACAAGTGCGGCTGCTGCTGGGAGCCCTGCAGTCTCTGCTGGGAACACAGCTGCCTC
CCCAGGGCAGAACCACCGCCCACAAGGACCCCAACGCCATCTTCCTGAGCTTCCAGCATCTGCTGAGA
GGCAAAGTGCGGTTCCTGATGCTCGTGGGCGGCAGCACACTGTGCGTGCGGAGAGCACCTCCTACCAC
AGCCGTGCCTAGCAGAACCAGCCTGGTGCTGACCCTGAACGAGCTGCCCAACAGAACCTCCGGCCTGC
TGGAAACAAACTTCACCGCCAGCGCCAGGACCACAGGCTCTGGACTGCTGAAGTGGCAGCAGGGCTT
CCGGGCCAAGATTCCTGGCCTGCTGAACCAGACCAGCAGAAGCCTGGACCAGATCCCCGGCTACCTG
AACCGGATCCACGAACTGCTGAACGGCACCAGAGGCCTGTTCCCAGGCCCCTCCAGAAGAACACTGG
GCGCTCCCGATATCAGCAGCGGCACCTCTGATACCGGCAGCCTGCCCCCTAATCTGCAGCCTGGCTAC
AGCCCTAGCCCTACCCACCCTCCAACCGGCCAGTACACCCTGTTCCCTCTGCCACCTACCCTGCCCACA
CCAGTGGTGCAGCTGCATCCTCTGCTGCCCGATCCTAGCGCCCCTACCCCTACACCAACAAGCCCCCT
GCTGAATACCAGCTACACCCACAGCCAGAACCTGAGCCAGGAAGGCTAA
```

1. A polypeptide comprising multiple domains, where at least two domains are selected from different members of the albumin superfamily.

2. The polypeptide of claim 1, wherein the members of the albumin superfamily from which each domain is selected are albumin, alpha-fetoprotein, vitamin D-binding protein and afamin.

3. (canceled)

4. The polypeptide of claim 1, wherein at least one domain is from vitamin D-binding protein.

5. (canceled)

6. The polypeptide of claim 5, wherein the polypeptide can bind vitamin D.

7. The polypeptide of claim 1, wherein at least one domain is selected from alphafetoprotein.

8. The polypeptide of claim 1, wherein at least one domain is selected from afamin.

9. (canceled)

10. (canceled)

11. (canceled)

12. The polypeptide of claim 1, wherein each domain of the polypeptide has 80% or greater homology to a domain selected from a member of the albumin superfamily.

13. The polypeptide of claim 1, wherein the entire polypeptide has less than 80% homology to human albumin or alpha-fetoprotein.

14. The polypeptide of claim 1, wherein each individual domain can comprise peptide sequences from more than one member of the human albumin superfamily.

15. The polypeptide of claim 14, wherein the domain can comprise one or more amino acid substitution when compared to the native domain from the albumin superfamily.

16. (canceled)

17. (canceled)

18. (canceled)

19. A polypeptide comprising the polypeptide of claim 1 and a protein of interest.

20. (canceled)

21. (canceled)

22. (canceled)

23. The polypeptide of claim 19, wherein the protein of interest is selected from the group consisting of coagulation factor IX, butyrylcholinesterase, coagulation factor VIII, coagulation factor VIIa, alpha-1-antitrypsin, antithrombin III, phenylalanine hydroxylase, erythropoietin, growth hormone, granulocyte colony stimulating factor, interferon beta, or atrial natriuretic peptide.

24. The polypeptide of claim 19, wherein the protein of interest is a vaccine antigen.

25. The polypeptide of claim 19, wherein the protein of interest is a single chain variable fragment.

26. The polypeptide of claim 19, wherein the protein of interest is a bispecific antibody.

27. A nucleic acid encoding the polypeptide of claim 1.

28. A nucleic acid encoding the polypeptide of claim 19.

29. (canceled)

30. The nucleic acid of claim 29, wherein the specific target sequence is the human albumin gene.

31. The nucleic acid of claim 29, wherein the target sequence is alpha-1-antitrypsin, transferrin, antithrombin III, alpha-fetoprotein, or insulin like growth factor II.

32. A vector comprising the nucleic acid of claim 27.

33. A host cell comprising the nucleic acid of claim 27.

34. (canceled)

35. (canceled)

36. (canceled)

37. (canceled)

38. The polypeptide of claim 1, wherein the polypeptide has 20% or greater half-life when compared to native albumin polypeptide.

39-85. (canceled)
